(19) United States
(12) Patent Application Publication
(10) Pub. No.: US 2023/0381315 A1
(43) Pub. Date: Nov. 30, 2023

(54) CELLS WITH CD70 KNOCKOUT AND USES FOR IMMUNOTHERAPY

(71) Applicants: MEMORIAL SLOAN-KETTERING CANCER CENTER, NEW YORK, NY (US); SLOAN-KETTERING INSTITUTE FOR CANCER RESEARCH, NEW YORK, NY (US); MEMORIAL HOSPITAL FOR CANCER AND ALLIED DISEASES, NEW YORK, NY (US)

(72) Inventors: Michel Sadelain, New York, NY (US); Jorge Mansilla-Soto, Forest Hills, NY (US); Sacha P. Haubner, New York, NY (US)

(73) Assignees: MEMORIAL SLOAN-KETTERING CANCER CENTER, NEW YORK, NY (US); SLOAN-KETTERING INSTITUTE FOR CANCER RESEARCH, NEW YORK, NY (US); MEMORIAL HOSPITAL FOR CANCER AND ALLIED DISEASES, NEW YORK, NY (US)

(21) Appl. No.: 18/306,890

(22) Filed: Apr. 25, 2023

Related U.S. Application Data
(63) Continuation of application No. PCT/US2021/056656, filed on Oct. 26, 2021.
(60) Provisional application No. 63/105,603, filed on Oct. 26, 2020.

(51) Int. Cl. A61K 39/00; A61P 37/02

(52) U.S. Cl. CPC A61K 39/4631; A61K 39/4621; A61P 37/02; A61K 39/464429

(57) ABSTRACT

The presently disclosed subject matter provides cells, compositions and methods for enhancing immune responses toward tumor antigens. It relates to cells comprising: an antigen-recognizing receptor (e.g., a chimeric antigen receptor, a TCR, or a TCR like fusion molecule); and a gene disruption of a CD70 locus. The gene disruption of the CD70 locus can improve the activity and/or efficiency of the cells.

### CROSS-REFERENCE TO RELATED APPLICATION

This application is a continuation application of International Patent Application No. PCT/US2021/056656, which claims priority to U.S. Provisional Application No. 63/105,603 filed on Oct. 26, 2020, the content of each of which is incorporated by reference in its entirety, and to each of which priority is claimed.

### SEQUENCE LISTING

A Sequence Listing conforming to the rules of WIPO Standard ST.26 is hereby incorporated by reference. Said Sequence Listing has been filed as an electronic document via PatentCenter in ASCII format encoded as XML. The electronic document, created on Apr. 25, 2023, is entitled “0727341444_ST26.xml”, and is 130,921 bytes in size.

### 1. INTRODUCTION

The presently disclosed subject matter provides cells, compositions and methods for enhancing immune responses toward tumor antigens. It relates to cells comprising an antigen-recognizing receptor (e.g., a chimeric antigen receptor (CAR) or a TCR like fusion molecule), and a gene disruption of a CD70 locus. The gene disruption of the CD70 locus can improve the activity and/or efficiency of the cells.

### 2. BACKGROUND OF THE INVENTION

Cell-based immunotherapy is a therapy with curative potential for the treatment of cancer. T cells and other immune cells may be modified to target tumor antigens through the introduction of genetic material coding for an antigen recognizing receptor, e.g., a Chimeric antigen receptor or a TCR like fusion molecule. Patient-engineered CAR T cells have demonstrated remarkable efficacy against a range of liquid and solid malignancies. However, treatment failure and relapses occur in a large fraction of patients. Therefore, there remain needs of improved immunotherapy.

### 3. SUMMARY OF THE INVENTION

The presently disclosed subject matter provides cells comprises: a) a first antigen-recognizing receptor that targets a first antigen; and b) a gene disruption of a CD70 locus. In certain embodiments, the gene disruption comprises a substitution, a deletion, an insertion, or a combination thereof. In certain embodiments, the mutation comprises s a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof.

In certain embodiments, the gene disruption of the CD70 locus results in a non-functional CD70 protein. In certain embodiments, the gene disruption of the CD70 locus results in knockout of the CD70 gene expression.

In certain embodiments, the gene disruption of the CD70 locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the cell is a cell of the lymphoid lineage or a cell of the myeloid lineage. In certain embodiments, the cell of the lymphoid lineage is selected from the group consisting of T cells, B cells, Natural Killer (NK) cells, and dendritic cells. In certain embodiments, the cell is a T cell. In certain embodiments, the T cell is derived from an induced pluripotent stem cell. In certain embodiments, the T cell is a CD8⁺ T cell. In certain embodiments, the CD8+ T cell is CD4 independent. In certain embodiments, the T cell is selected from the group consisting of a cytotoxic T lymphocyte (CTL), a γδ T cell, a tumor-infiltrating lymphocyte (TIL), a regulatory T cell, and a Natural Killer T (NKT) cell.

In certain embodiments, the first antigen-recognizing receptor is integrated at a locus within the genome of the T cell. In certain embodiments, the locus is selected from the group consisting of a TRAC locus, a TRBC locus, a TRDC locus, and a TRGC locus. In certain embodiments, the locus is a TRAC locus or a TRBC locus. In certain embodiments, the locus is a TRAC locus.

In certain embodiments, the cell further comprises a second antigen-recognizing receptor that targets a second antigen.

In certain embodiments, the first antigen and/or the second antigen is a tumor antigen or a pathogen antigen. In certain embodiments, the tumor antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the first antigen and/or the second antigen is expressed on an acute myeloid leukemia (AML) tissue.

In certain embodiments, the first antigen and/or the second antigen is expressed on an acute myeloid leukemia (AML) hematopoietic stem/progenitor cell (HSPC) and/or a leukemia stem cell (LSC). In certain embodiments, the AML HSPC and/or LSC expresses CD34. In certain embodiments, the first antigen and/or the second antigen is not expressed or expressed at a non-detectable level in a non-malignant hematopoietic stem cell and/or a non-malignant hematopoietic progenitor cell. In certain embodiments, the first antigen and/or the second antigen is independently selected from the group consisting of CD19, CD70, IL1RAP, CD33, CLEC12A, ADGRE2, CD123 and combinations thereof. In certain embodiments, the first antigen is CD19. In certain embodiments, the first antigen is CD70. In certain embodiments, the first antigen and the second antigen are CD70 and IL1RAP.

In certain embodiments, the first antigen-recognizing receptor is a chimeric antigen receptor (CAR), a T cell receptor (TCR), or a TCR like fusion molecule.

In certain embodiments, the first antigen-recognizing receptor is a TCR like fusion molecule. In certain embodiments, the TCR like fusion molecule is a recombinant TCR that comprises i) a first antigen-binding chain comprising an antigen-binding fragment of a heavy chain variable region (V_{H}) of an antibody; and ii) a second antigen-binding chain comprising an antigen-binding fragment of a light chain variable region (V_{L}) of the antibody; wherein the first and second antigen-binding chains a) each comprise a TRAC polypeptide or a TRBC polypeptide, and b) bind to the first antigen. In certain embodiments, the recombinant TCR binds to the first antigen in an HLA-independent manner. In certain embodiments, the first antigen is CD70. In certain embodiments, the TRAC polypeptide is a native TRAC polypeptide or a modified TRAC polypeptide. In certain embodiments, the TRBC polypeptide is a native TRBC polypeptide or modified TRBC polypeptide. In certain embodiments, at least one of the TRAC polypeptide and the TRBC polypeptide is endogenous.

In certain embodiments, the first and the second antigen-binding chains bind to the first antigen with a dissociation constant (K_{D}) of about 1×10⁻⁸ M or less. In certain embodiments, the first and the second antigen-binding chains bind to the first antigen with a dissociation constant (K_{D}) of about 5×10⁻⁹ M or less.

In certain embodiments, the first antigen-binding chain comprises an antigen-binding fragment of a V_{H} of an antibody and a TRBC polypeptide, and the second antigen-binding chain comprises an antigen-binding fragment of a V_{L} of the antibody and a TRAC polypeptide.

In certain embodiments, the first antigen-binding chain comprises an antigen-binding fragment of a V_{H} of an antibody and a TRAC polypeptide, and the second antigen-binding chain comprises an antigen-binding fragment of a V_{L} of the antibody and a TRBC polypeptide.

In certain embodiments, the first antigen binding chain and the second antigen binding chains are capable of associating with a CD3ζ polypeptide. In certain embodiments, the first antigen binding chain and the second antigen binding chains, upon binding to the first antigen, are capable of activating the CD3ζ polypeptide. In certain embodiments, the activation of the CD3ζ polypeptide is capable of activating the cell.

In certain embodiments, the recombinant TCR is integrated at a TRAC locus. In certain embodiments, the cell further comprises a gene disruption of a TRBC locus.

In certain embodiments, the first antigen-recognizing receptor is a chimeric receptor. In certain embodiments, the chimeric receptor is a chimeric antigen receptor (CAR).

In certain embodiments, the second antigen-recognizing receptor is a chimeric antigen receptor (CAR) or a chimeric co-stimulating receptor (CCR).

In certain embodiments, the second antigen-recognizing receptor is a chimeric antigen receptor (CAR).

In certain embodiments, any of the CAR disclosed herein comprises an extracellular antigen-binding domain that binds to the second antigen, and an intracellular signaling domain that is capable of delivering an activation signal to the cell. In certain embodiments, the intracellular signaling domain of the CAR comprises a CD3ζ polypeptide. In certain embodiments, the CD3ζ polypeptide is a native CD3ζ polypeptide or a modified CD3ζ polypeptide. In certain embodiments, the modified CD3ζ polypeptide comprises a native ITAM1, an ITAM2 variant consisting of two loss-of-function mutations, and an ITAM3 variant consisting of two loss-of-function mutations. In certain embodiments, the intracellular signaling domain of the CAR further comprises at least one costimulatory signaling region. In certain embodiments, the at least one costimulatory signaling region comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D. In certain embodiments, the CAR comprises a transmembrane domain.

In certain embodiments, the second antigen-recognizing receptor is a CCR. In certain embodiments, the CCR comprises an extracellular antigen-binding domain that binds to the second antigen and an intracellular domain that is capable of delivering a costimulatory signal to the cell but does not alone deliver an activation signal to the cell. In certain embodiments, the intracellular domain of the CCR comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the cell is a T cell, and the first antigen-recognizing receptor and the second antigen-recognizing receptor are integrated into a TRAC locus of the T cell.

In certain embodiments, the cell is a T cell, the first antigen-recognizing receptor is integrated into a TRAC locus of the T cell, and the second antigen-recognizing receptor is integrated into the CD70 locus.

In certain embodiments, the cell is a T cell, the first antigen-recognizing receptor is integrated into the CD70 locus, and the second antigen-recognizing receptor is integrated into the TRAC locus of the T cell.

In certain embodiments, the cell is a T cell, and the first antigen-recognizing receptor and the second antigen-recognizing receptor are integrated into a TRBC locus of the T cell.

In certain embodiments, the cell is a T cell, the first antigen-recognizing receptor is integrated into a TRBC locus of the T cell, and the second antigen-recognizing receptor is integrated into the CD70 locus.

In certain embodiments, the cell is a T cell, the first antigen-recognizing receptor is integrated into the CD70 locus, and the second antigen-recognizing receptor is integrated into the TRBC locus of the T cell.

In certain embodiments, the cell further comprises at least one exogenous costimulatory ligand. In certain embodiments, the at least one exogenous co-stimulatory ligand is selected from the group consisting of a tumor necrosis factor (TNF) family member, an immunoglobulin (Ig) superfamily member, and combinations thereof. In certain embodiments, the TNF family member is selected from the group consisting of 4-1BBL, OX40L, CD70, FasL, GITRL, TNF-related apoptosis-inducing ligand (TRAIL), CD30L, LIGHT (TNFSF14), CD40L, and combinations thereof. In certain embodiments, the Ig superfamily member is selected from the group consisting of CD80, CD86, ICOSLG, and combinations thereof. In certain embodiments, the at least one exogenous a costimulatory ligand comprises CD80. In certain embodiments, the at least one exogenous a costimulatory ligand comprises 4-1BBL. In certain embodiments, the cell comprises two exogenous costimulatory ligands. In certain embodiments, the at least two exogenous costimulatory ligands comprise CD80 and 4-1BBL.

In certain embodiments, the cell further comprises a fusion polypeptide that comprises: a) an extracellular domain and a transmembrane domain of a co-stimulatory ligand, and b) an intracellular domain of a first co-stimulatory molecule. In certain embodiments, the co-stimulatory ligand is selected from the group consisting of a tumor necrosis factor (TNF) family member, an immunoglobulin (Ig) superfamily member, and combinations thereof. In certain embodiments, the TNF family member is selected from the group consisting of 4-1BBL, OX40L, CD70, GITRL, CD40L, and combinations thereof. In certain embodiments, the Ig superfamily member is selected from the group consisting of CD80, CD86, ICOSLG, and combinations thereof. In certain embodiments, the co-stimulatory ligand is CD80.

In certain embodiments, the first co-stimulatory molecule is selected from the group consisting of CD28, 4-1BB, OX40, ICOS, DAP-10, CD27, CD40, NKG2D, CD2, CD150, CD226, and combinations thereof. In certain embodiments, the first co-stimulatory molecule is 4-1BB.

In certain embodiments, the co-stimulatory ligand is CD80 and the first co-stimulatory molecule is 4-1BB. In certain embodiments, the fusion polypeptide further comprises an intracellular domain of a second co-stimulatory molecule. In certain embodiments, the second co-stimulatory molecule is selected from the group consisting of CD28, 4-1BB, OX40, ICOS, DAP-10, CD27, CD40, NKG2D, CD2, CD150, CD226, and combinations thereof. In certain embodiments, the second co-stimulatory molecule is CD28. In certain embodiments, the co-stimulatory ligand is CD80, the first co-stimulatory molecule is 4-1BB, and the second co-stimulatory molecule is CD28.

In certain embodiments, the cell further comprises a gene disruption of a B2M locus. In certain embodiments, the gene disruption of the B2M locus results in a non-functional beta 2-microglobulin. In certain embodiments, the gene disruption of the B2M locus results in knockout of the B2M gene expression. In certain embodiments, the gene disruption of the B2M locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the CD70 locus is capable of enhancing at least one activity of the cell comprising the first antigen-recognizing receptor. In certain embodiments, the at least one activity comprises cytotoxicity, cell proliferation, cell persistence, or a combination thereof.

In certain embodiments, the cell is autologous. In certain embodiments, the cell is allogeneic.

The presently disclosed subject matter provides compositions comprising the cells disclosed herein. In certain embodiments, the composition is a pharmaceutical composition further comprising a pharmaceutically acceptable excipient.

The presently disclosed subject matter further provides methods for producing the presently disclosed cells. In certain embodiments, the method comprising generating a gene disruption of a CD70 locus in a cell comprising the presently disclosed first antigen-recognizing receptor. In certain embodiments, the gene disruption comprises a substitution, a deletion, an insertion, or a combination thereof. In certain embodiments, the mutation comprises s a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof. In certain embodiments, the gene disruption of the CD70 locus results in a non-functional CD70 protein. In certain embodiments, the gene disruption of the CD70 locus results in knockout of the CD70 gene expression. In certain embodiments, generating the gene disruption of the CD70 locus comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the cell further comprises a second antigen-recognizing receptor that binds to the second antigen. In certain embodiments, the second antigen recognizing receptor is a chimeric antigen receptor or a chimeric co-simulating receptor (CCR). In certain embodiments, the cell further comprises at least one exogenous costimulatory ligand. In certain embodiments, the cell further comprises a fusion polypeptide comprising: a) an extracellular domain and a transmembrane domain of a co-stimulatory ligand, and b) an intracellular domain of a first co-stimulatory molecule. In certain embodiments, the method further comprises generating a gene disruption of a B2M locus. In certain embodiments, the gene disruption of the B2M locus results in a non-functional beta 2-microglobulin. In certain embodiments, the gene disruption of the CD70 locus results in knockout of the B2M gene expression. In certain embodiments, generating the gene disruption of the B2M locus to the cell comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof. The presently disclosed subject matter also provides cells produced by the presently disclosed methods.

Furthermore, the presently disclosed subject matter provides methods of reducing tumor burden in a subject. In certain embodiments, the method comprises administering to the subject an effective amount of the cells or the composition disclosed herein. In certain embodiments, the method reduces the number of tumor cells, reduces tumor size, and/or eradicates the tumor in the subject.

Furthermore, the presently disclosed subject matter provides methods of treating and/or preventing a tumor or a neoplasm in the subject. In certain embodiments, the method comprises administering to the subject an effective amount of the cells or the composition disclosed herein.

In certain embodiments, the neoplasm or tumor is cancer. In certain embodiments, the neoplasm or tumor is a solid tumor. In certain embodiments, the neoplasm or tumor is a blood cancer. In certain embodiments, the neoplasm or tumor is a myeloid disorder. In certain embodiments, the myeloid disorder is selected from the group consisting of myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera. In certain embodiments, the myeloid disorder is acute myeloid leukemia (AML).

Furthermore, the presently disclosed subject matter provides methods of preventing and/or treating a pathogen infection in the subject. In certain embodiments, the method comprises administering to the subject an effective amount of the cells or the composition disclosed herein.

The presently disclosed subject matter further provides methods preventing and/or treating an autoimmune disease in a subject. In certain embodiments, the method comprises administering to the subject an effective amount of the cells or the composition disclosed herein.

The presently disclosed subject matter further provides methods preventing and/or treating an infectious disease in a subject. In certain embodiments, the method comprises administering to the subject an effective amount of the cells or the composition disclosed herein. In certain embodiments, the method further comprises administering to the subject a CD70-targeted therapy. In certain embodiments, the subject receives a CD70-targeted therapy.

Furthermore, the presently disclosed subject matter provides kits comprising the cell cells disclosed herein or the composition disclosed herein. In certain embodiments, the kit further comprises written instructions for reducing tumor burden and/or treating and/or preventing a neoplasm (e.g., a cancer), a pathogen infection, an autoimmune disease, and/or an infectious disease.

### 4. BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A and 1B depict CD70 expression on 1928z CAR T cells. Un-stimulated T cells were electroporated either with CD70 gRNA/Cas9 RNP (FIG. 1A) or without CD70 gRNA/Cas9 RNP (FIG. 1B). This was followed by CD3/CD28-bead activation for 48 hours. After bead removal, T cells were transduced with a 1928z γ-retroviral vector. CD70 expression was evaluated 48h post-transduction: control CAR T cells (FIG. 1B) show >50% of CD70+ cells; in contrast only 1.4% of CRISPR-edited CAR-T cells (FIG. 1A) showed low levels of CD70, demonstrating efficient CD70 gene-editing.

FIGS. 2A-2D depict CD70 knock-out enhanced CD70-CAR T cell activity. FIG. 2A depicts the results of an in vitro cytotoxicity assay comparing un-transduced (UTD) T cells vs. 7-retrovirally-transduced CD70-28z1XX CAR T cells with TRAC KO+/−CD70 KO. FIG. 2B depicts the results of an in-vivo cytotoxicity assay comparing un-transduced (UTD) T cells vs. 7-retrovirally-transduced CD70-28z1XX CAR T cells with TRAC KO+/−CD70 KO. For in-vivo assay, NSG mice were injected with a solid tumor cell line on d-5 (7×10⁵ tumor cells/mouse via tail-vein) and treated with T cells on d0 (1×10⁶ CAR+ T cells or equivalent number of UTD T cells via tail-vein). On d26 after T cell administration, bone marrow was assessed for absolute numbers of CD4+ CAR+ T cells (FIG. 2C) and CD8+ CAR+ T cells (FIG. 2D).

FIGS. 3A and 3B depict that multiplex gene-edited CD70-KO CD70-HIT+ costim T cells outperformed CD70-28z1XX-CAR T cells in AML. FIG. 3A depicts the results of an in vitro cytotoxicity assay comparing different CD70-HIT vs. CD70-CAR formats. FIG. 3B depicts the results of an in vivo NSG xenograft cytotoxicity assay comparing different CD70-HIT vs. CD70-CAR formats. For the in vitro, CD70-HIT allowed for higher AML killing efficiency compared to second-generation CAR formats (both zeta-WT and zeta-1XX CAR), shown in an 18h cytotoxicity assay. For the in vivo assay, NSG mice were injected with an AML cell line on d-4 (1×10⁶ AML cells/mouse via tail-vein) and treated with T cells on d0 (4×10⁵ CAR/HIT+ T cells). The additional co-stimulation signal was provided by co-stimulatory ligands CD80 and 4-1BBL. “CD70-KO CD70-HIT+80/BBL” represent T cells comprising a knockout of a CD70 locus, a CD70-HIT, an exogenous CD80, and an exogenous 4-1BBL.

FIGS. 4A and 4B depict restricted expression profile of CD70 and IL1RAP in normal hematopoiesis. FIG. 4A depicts the transcriptional expression profile of CD70 and FIG. 4B depicts the transcriptional expression profile of IL1RAP, both of which are illustrated as hierarchical differentiation tree of normal hematopoiesis (small blue dots=negativity, large red dots=positivity). Green circles highlight normal hematopoietic stem/progenitor cell population, indicating negativity for both CD70 and IL1RAP on this vital cell population. Data and figure generated using publicly available transcriptome database (bloodspot.eu).

FIG. 5 depicts restricted expression profile of CD70 and IL1RAP in non-hematopoietic normal tissues. Protein expression profile of CD70, IL1RAP, other reported AML surface target antigens and CD19 as reference target (yellow color indicates high protein expression level, violet color indicates absent protein expression, grey color indicates missing data). Green rectangle highlights favorable normal tissue expression profile of CD70 and IL1RAP with negativity in most normal tissues, in contrast to some other reported AML targets, e.g. CD135 (FLT3) and FOLR2 (folate receptor beta). Data and figure were generated using an integrated dataset including 3 publicly available proteomic data repositories: Human Proteome Map (HPM) (www.humanproteomemap.org), Proteomics Database (PDB) (www.proteomicsdb.org) and a deep proteome abundance atlas (Wang et al. Molecular Systems Biology 2019). Integration of proteomics repositories is originally based on methodology published in Perna et al. Cancer Cell. (2017 Oct. 9); 32(4):506-519.

FIGS. 6A-6C depict gene-editing approach for targeted integration and knockout (“KO”) in accordance with certain embodiments of the presently disclosed subject matter. FIG. 6A depicts integration of a TCR like fusion molecule (e.g., HIT) into a TRAC locus. FIG. 6B depicts genetic strategies to generate a second knock-in, e.g., a CAR or a CCR expression cassette targeted to the first exon of the CD70 gene. FIG. 6C depicts gene-editing strategy to generate B2M-KO. CRISPR/Cas9 can be targeted to the first exon of the B2M gene (as shown in Eyquem et al., Nature (2017); 543:113-117).

FIGS. 7A and 7B depict CD70 expression levels for MOLM13 cells and in vitro cytotoxic activity of TRAC-HIT T cells and RV-CAR T cells on MOLM13 cells. FIG. 7A shows CD70 flow histograms for MOLM13 cells in vitro and ex-vivo (from mouse bone marrow samples), and non-activated T cells (negative control). FIG. 7B shows cytotoxic activity using an 18-h bioluminescence assay at different effector:target ratios for TRAC-70HIT/RV-CAR T cells (7028z and 7028z1xx) and FFL+ MOLM13 cells.

FIG. 8 shows cytotoxic activity of 70HIT T cells using an 18 h bioluminescence assay, using firefly luciferase (FFL)-expressing targets cells. CD70 gene was CRISPR/Cas9-edited in MOLM13 cell line. 70 HIT T cells selectively lysed CD70-expressing target cells only. All data are mean±s.e.m.

FIGS. 9A-9C depict CD70 knock-out enhanced CD70-CAR T cell activity. FIG. 9A shows Kaplan-Meier analysis of survival of MOLM13-bearing mice treated with 4×10⁵ TRAC-HIT or RV-CAR T cells (n=5). FIGS. 9B and 9C show tumor burden (average radiance) of MOLM13-bearing mice treated with 4×10⁵ TRAC-HIT or RV-CAR T cells (n=5), analyzed through a 35-day period.

### 5. DETAILED DESCRIPTION OF THE INVENTION

The presently disclosed subject matter provides cells with enhanced activity and efficacy for immunotherapy (e.g., T cell immunotherapy). The presently disclosed cells comprise a gene disruption of a CD70 locus and an antigen-recognizing receptor targeting a first antigen, e.g., a chimeric receptor (e.g., a chimeric antigen receptor (CAR)) or a TCR like fusion molecule (e.g., a HIT CAR). To provide additional stimulation to the cells, the cells can further comprise a) a second antigen-recognizing receptor targeting a second antigen (e.g., a chimeric co-stimulating receptor (CCR) or a CAR), b) at least one exogenous costimulatory ligand, and/or c) a fusion polypeptide comprising an extracellular domain and a transmembrane domain of a co-stimulatory ligand, and at least one intracellular domain of a co-stimulatory molecule. The gene disruption of the CD70 locus can result in a non-functional CD70 protein or knockout of the CD70 gene expression. The presently disclosed subject matter also provides methods for producing such cells, and methods of using such cells for treating and/or preventing tumor (e.g., cancer, e.g., a solid tumor or a blood cancer, e.g., a myeloid disorder, e.g., acute myeloid leukemia (AML)). The presently disclosed subject matter is based, at least in part, on the discovery that a CD70 is highly expressed on a substantial fraction of T cells comprising an antigen-recognizing receptor (e.g., a CAR), and knockout of CD70 can enhance at least one activity of the cells, e.g., cytotoxicity, cell proliferation, and/or cell persistence.

Non-limiting embodiments of the presently disclosed subject matter are described by the present specification and Examples.

For purposes of clarity of disclosure and not by way of limitation, the detailed description is divided into the following subsections:
5.1. Definitions;
5.2. Gene Disruption of A CD70 Locus;
5.3. Cells;
5.4. Compositions and Vectors;
5.5. Formulations and Administration;
5.6. Methods of Treatment; and
5.7. Kits.

### 5.1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, a “co-stimulatory molecule” refer to a cell surface molecule other than an antigen receptor or its ligand that can provide an efficient response of lymphocytes to an antigen. In certain embodiments, a co-stimulatory molecule can provide optimal lymphocyte activation.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40, ICOS, DAP-10, CD27, NKG2D, CD2, CD150, CD226. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain variable fragment (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant CL region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U.S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs regions are delineated using the PyIgClassify system (Adolf-Bryfogle et al., Nucleic acids research 43.D1 (2015): D432-D438).

As used herein, the term “Linker” shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker is a G4S linker. In certain embodiments, the linker comprises or consists of the amino acid sequence set forth in SEQ ID NO: 10, which is provided below:

[SEQ ID NO: 10]
GGGGSGGGGSGGGGS

In certain embodiments, the linker comprises or consists of the amino acid sequence set forth in SEQ ID NO: 59, which is provided below:

[SEQ ID NO: 59]
GGGGSGGGGSGGGSGGGGS

In certain embodiments, the linker comprises or consists of the amino acid sequence set forth in SEQ ID NO: 60, which is provided below:

[SEQ ID NO: 60]
GGGGSGGGGSGGGGSGGGSGGGGS

In certain embodiments, the linker comprises or consists of the amino acid sequence set forth in SEQ ID NO: 61, which is provided below:

[SEQ ID NO: 61]
GGGGSGGGGSGGGGSGGGGSGGGSGGGGS

In certain embodiments, the linker comprises or consists of the amino acid sequence set forth in SEQ ID NO: 62, which is provided below:

[SEQ ID NO: 62]
GGGGS

In certain embodiments, the linker comprises or consists of the amino acid sequence set forth in SEQ ID NO: 63, which is provided below:

[SEQ ID NO: 63]
GGGGSGGGGS

As use herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife et al., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv703) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed CD19-targeted CAR (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” is meant a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

By “specifically binds” is meant a polypeptide or a fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor or capable of suppressing an immune response via receptor-ligand binding.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

Other aspects of the presently disclosed subject matter are described in the following disclosure and are within the ambit of the presently disclosed subject matter.

### 5.2. Gene Disruption of A CD70 Locus

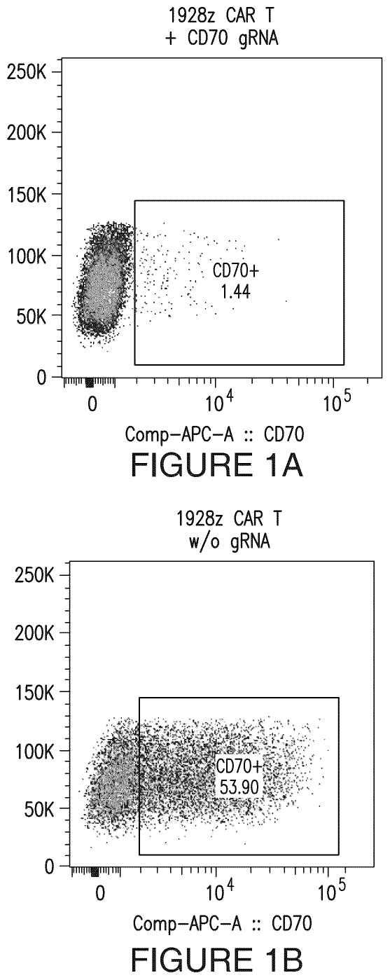

The presently disclosed subject matter provides cells comprising a gene disruption of a CD70 locus. The inventors discovered that CD70 is highly expressed in T cells comprising an antigen-recognizing receptor (e.g., a chimeric antigen receptor (CAR) (see FIGS. 1A and 1B) or a TCR like fusion molecule, a HIT CAR), which can lead to T cell fratricide or suicide, thereby impairing the efficiency of the cells for immunotherapy. Therefore, a gene disruption of a CD70 locus in cells comprising an antigen-recognizing receptor can improve at least one activity of the cells, e.g., cytotoxicity, cell proliferation, and/or cell persistence. In certain embodiments, the antigen-recognizing receptor binds to CD70. In certain embodiments, the immunotherapy further comprises a CD70-targeted therapy.

The gene disruption of the CD70 locus can result in a non-functional CD70 protein or a knockout of the CD70 gene expression. In certain embodiments, the gene disruption of the CD70 locus results in knockout of the CD70 gene expression.

Non-limiting examples of gene disruptions include substitutions, deletions, insertions, or combinations thereof. In certain embodiments, the mutation comprises a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof.

In certain embodiments, the CD70 locus is a human CD70 locus. The gene disruption of the CD70 locus can be generated by any suitable gene editing methods. In certain embodiments, the gene disruption of the CD70 locus (e.g., knockout of the CD70 locus) is generated using a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adena-associated viral vectors, vaccinia viruses, bovine papilloma viruses, and herpes viruses (e.g., such as Epstein-Barr Virus).

In certain embodiments, the gene disruption of the CD70 locus (e.g., knockout of the CD70 locus) is generated using a non-viral method. Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to generate the gene disruption of the CD70 locus. In certain embodiments, the gene disruption of the CD70 locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, a CRISPR system is used to generate the gene disruption of the CD70 locus.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. In certain embodiments, the CRISPR system comprises base editors. In certain embodiments, the CRISPR system comprises transposases/recombinases. In certain embodiments, the CRISPR system comprises prime editors. In certain embodiments, the CRISPR system comprises an epigenetic modulator. In certain embodiments, the CRISPR system comprises is a CRISPRoff system. Additional details on the CRISPR systems of the presently disclosed subject matter can be found in Anzalone et al., Nature biotechnology 38.7 (2020): 824-844 and in Nun\tilde{e}z et al., Cell 184.9 (2021): 2503-2519, the contents of each of which are incorporated by reference in their entireties.

In certain embodiments, the CD70 locus is disrupted using a gRNA molecule to knockout expression of CD70. The gRNA molecule can target a coding sequence of a CD70 gene (e.g., a human CD70 gene) or a non-coding sequence of a CD70 gene (e.g., a human CD70 gene). In certain embodiments, the gRNA molecule targets a coding sequence of a CD70 gene (e.g., a human CD70 gene). In certain embodiments, the gRNA molecule targets a target sequence within a human CD70 gene. In certain embodiments, the target sequence comprises or consists of the nucleotide sequence set forth in SEQ ID NO: 82. In certain embodiments, the gRNA molecule comprises or consists of the nucleotide sequence set forth in SEQ ID NO: 68. SEQ ID NOs: 68 and 82 are provided below:

[SEQ ID NO: 68]
GGGCUUGGUGAUCUGCCUCG
[SEQ ID NO: 82]
GGGCTTGGTGATCTGCCTCG

In certain embodiments, zinc-finger nucleases are used to generate the gene disruption of the CD70 locus. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, a TALEN system is used to generate the gene disruption of the CD70 locus. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the gene disruption of the CD70 locus can be a disruption of the coding region of the CD70 locus and/or a disruption of the non-coding region of the CD70 locus. In certain embodiments, the gene disruption of the CD70 locus comprises a disruption of the coding region of the CD70 locus. In certain embodiments, the gene disruption of the CD70 locus comprises an insertion at the coding region of the CD70 locus. Human CD70 protein comprises three exons: exon 1, exon 2, and exon 3. In certain embodiments, the gene disruption of the CD70 locus comprises a disruption at one or more of exon 1, exon 2, and exon 3 of the CD70 locus. In certain embodiments, the gene disruption of the CD70 locus comprises a disruption at exon 1 of the CD70 locus. In certain embodiments, the gene disruption of the CD70 locus comprises an insertion at exon 1 of the CD70 locus.

In certain embodiments, the gene disruption of the CD70 locus is produced prior to the expression of the first antigen-recognizing receptor in the cell.

### 5.3. Cells

The presently disclosed subject matter provides cells comprising a) a gene disruption of a CD70 locus (e.g., one disclosed in Section 5.2) and b) a first antigen-recognizing receptor that targets a first antigen.

In certain embodiments, the cell is selected from the group consisting of cells of lymphoid lineage and cells of myeloid lineage. In certain embodiments, the cell is an immunoresponsive cell.

In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

In certain embodiments, the cell is a NK cell. Natural Killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the cell (e.g., T cell) is autologous. In certain embodiments, the cell (e.g., T cell) is non-autologous. In certain embodiments, the cell (e.g., T cell) is allogeneic. In certain embodiments, the cell (e.g., T cell) is derived in vitro from an engineered progenitor or stem cell.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

5.3.1. First Antigen-Recognizing Receptors

The first antigen-recognizing receptor targets a first antigen. The first antigen can be a tumor antigen or a pathogen antigen. In certain embodiments, the first antigen-recognizing receptor is a chimeric receptor. In certain embodiments, the chimeric receptor is a chimeric antigen receptor (CAR). In certain embodiments, the first antigen-recognizing receptor is a TCR like fusion molecule. In certain embodiments, the first antigen-recognizing receptor is a T Cell Receptor (TCR).

5.3.1.1. First Antigen

In certain embodiments, the first antigen is a tumor antigen. Any tumor antigen (antigenic peptide) can be used in the tumor-related embodiments described herein. Sources of antigen include, but are not limited to, cancer proteins. The first antigen can be expressed as a peptide or as an intact protein or a portion thereof. The intact protein or portion thereof can be native or mutagenized. Non-limiting examples of tumor antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, K-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the first antigen is CD19. In certain embodiments, the first antigen is expressed on an acute myeloid leukemia (AML) tissue. In certain embodiments, the first antigen is expressed on an acute myeloid leukemia (AIL) hematopoietic stem/progenitor cell (HSPC) and/or a leukemia stem cell (LSC). In certain embodiments, the AML HSPC expresses CD34. In certain embodiments, the first antigen is expressed in a malignant hematopoietic stem cell and/or a malignant hematopoietic progenitor cell. In certain embodiments, the first antigen is not expressed or expressed at a non-detectable level in a non-malignant hematopoietic stem cell and/or a non-malignant hematopoietic progenitor cell. In certain embodiments, the first antigen is selected from the group consisting of CD70, IL1RAP, CD19, CD33, CLEC12A, ADGRE2, CD123, and combinations thereof. In certain embodiments, the first antigen is CD70.

In certain embodiments, the first antigen is a pathogen antigen. Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g. M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, corynebacterium sp., Erysipelothrix rhusiopathiae, Clostridium spp., Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

5.3.1.2. Chimeric Receptors

In certain embodiments, the first antigen-recognizing receptor is a chimeric receptor. In certain embodiments, the chimeric receptor is a chimeric antigen receptor (CAR). In certain embodiments, the chimeric receptor that comprises a ligand or a portion thereof that binds to the first antigen (referred to as “chimeric ligand receptor”)

5.3.1.2.1 Chimeric Antigen Receptors (CARs)

CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs include a signaling domain of a co-stimulatory molecule (e.g., CD28, 4-1BB, ICOS, OX40, CD27, CD40, NKG2D, DAP-10, CD2, CD150, CD226) to the intracellular signaling domain of the CAR to provide co-stimulation signals to the cell (e.g., T cell or NK cell). “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ).

In certain embodiments, the first antigen-recognizing receptor is a CAR comprising an extracellular antigen-binding domain that binds to the first antigen, and an intracellular signaling domain. In certain embodiments, the CAR further comprises a transmembrane domain. In certain embodiments, the CAR further comprises a finger/spacer region.

In certain embodiments, the extracellular antigen-binding domain of the CAR (for example, an scFv) binds to the first antigen with a dissociation constant (K_{D}) of about 5×10⁻⁷ M or less, about 1×10⁻⁷ M or less, about 5×10⁻⁸ M or less, about 1×10⁻⁸ M or less, about 5×10⁻⁹ M or less, or about 1×10⁻⁹ M or less, or about 1×10⁻¹⁰ M or less. In certain embodiments, the extracellular antigen-binding domain of the CAR (for example, an scFv) binds to the first antigen with a K_{D} of about 1×10⁻⁸ M or less. In certain embodiments, the extracellular antigen-binding domain of the CAR (for example, an scFv) binds to the first antigen with a K_{D} of about 2×10⁻⁹ M.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

The extracellular antigen-binding domain can comprise or be an scFv, a Fab (which is optionally crosslinked), or a F(ab)₂. In certain embodiments, any of the foregoing molecules may be comprised in a fusion protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

In certain embodiments, the first antigen-recognizing receptor is a CAR comprising an extracellular antigen-binding domain that binds to CD70. In certain embodiments, the extracellular antigen-binding domain of the CAR comprises or consists of the amino acid sequence set forth in SEQ ID NO: 9 and specifically binds to CD70, e.g., a human CD70 polypeptide. SEQ ID NO: 9 is provided in Table 1 below.

In certain embodiments, the extracellular antigen-binding domain of the CAR comprises a heavy chain variable region (V_{H}) comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. In certain embodiments, the extracellular antigen-binding domain of the CAR comprises a V_{H} comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3.

In certain embodiments, the extracellular antigen-binding domain of the CAR comprises a light chain variable region (V_{L}) comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. In certain embodiments, the extracellular antigen-binding domain of the CAR comprises a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6. SEQ ID NOs: 4-6 are provided in Table 1 below.

In certain embodiments, the extracellular antigen-binding domain of the CAR comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3, a conservative modification thereof, a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. In certain embodiments, the extracellular antigen-binding domain of the CAR comprises a V_{H} comprising a CDR1 comprising amino acids having the sequence set forth in SEQ ID NO: 1, a CDR2 comprising the amino acid sequence set forth in SEQ ID NO: 2, and a CDR3 comprising the amino acid sequence set forth in SEQ ID NO: 3; and a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 7. For example, the extracellular antigen-binding domain of the first antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 7. In certain embodiments, the extracellular antigen-binding domain of the CAR comprises a V_{H} comprising the amino acid sequence set forth in SEQ ID NO: 7. SEQ ID NO: 7 is provided in Table 1 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 8. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 8. In certain embodiments, the extracellular antigen-binding domain of the CAR comprises a V_{L} comprising the amino acid sequence set forth in SEQ ID NO: 8. SEQ ID NO: 8 is provided in Table 1 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 7, and a V_{L} comprising an amino acid sequence that is at least about 80% (e.g., at least about 85%, at least about 90%, or at least about 95%) homologous or identical to the amino acid sequence set forth in SEQ ID NO: 8.

In certain embodiments, the extracellular antigen-binding domain of the CAR comprises a V_{H} comprising the amino acid sequence set forth in SEQ ID NO: 7. In certain embodiments, the extracellular antigen-binding domain of the CAR comprises a V_{L} comprising the amino acid sequence set forth in SEQ ID NO: 8. In certain embodiments, the extracellular antigen-binding domain of the CAR comprises V_{H} comprising the amino acid sequence set forth in SEQ ID NO: 7 and a V_{L} comprising the amino acid sequence set forth in SEQ ID NO: 8.

In certain embodiments, the extracellular antigen-binding domain of the CAR comprises a CDR1, a CDR2, and a CDR3 of a V_{H} sequence of an anti-CD70 antibody disclosed in International Patent Publication No. WO 2007/038637, which is incorporated by reference in its entirety. In certain embodiments, the extracellular antigen-binding domain of the CAR comprises a CDR1, a CDR2, and a CDR3 of the V_{H} sequence of the anti-CD70 antibody 2H5 disclosed in International Patent Publication No. WO 2007/038637. In certain embodiments, the extracellular antigen-binding domain of the CAR comprises a V_{H} sequence of an anti-CD70 antibody disclosed in International Patent Publication No. WO 2007/038637.

In certain embodiments, the extracellular antigen-binding domain of the CAR comprises a CDR1, a CDR2, and a CDR3 of a V_{L} sequence of an anti-CD70 antibody disclosed in International Patent Publication No. WO 2007/038637. In certain embodiments, the extracellular antigen-binding domain of the CAR comprises a CDR1, a CDR2, and a CDR3 of the V_{L} sequence of the anti-CD70 antibody 2H5 disclosed in International Patent Publication No. WO 2007/038637. In certain embodiments, the extracellular antigen-binding domain of the CAR comprises a V_{L} sequence of an anti-CD70 antibody disclosed in International Patent Publication No. WO 2007/038637.

In certain embodiments, the extracellular antigen-binding domain of the CAR comprises an antigen-binding fragment of an anti-CD70 antibody disclosed in International Patent Publication No. WO 2007/038637. In certain embodiments, the extracellular antigen-binding domain of the CAR comprises an antigen-binding fragment of the anti-CD70 antibody 2H5 disclosed in International Patent Publication No. WO 2007/038637.

In certain embodiments, the extracellular antigen-binding domain of the CAR comprises an antigen-binding fragment of an anti-CD70 antibody disclosed in International Patent Publication No. WO 2007/038637. In certain embodiments, the extracellular antigen-binding domain of the CAR comprises an antigen-binding fragment of the anti-CD70 antibody 2H5 disclosed in International Patent Publication No. WO 2007/038637.

In certain embodiments, the extracellular antigen-binding domain of the CAR is an scFv that comprises or consists of the amino acid sequence set forth in SEQ ID NO: 9. SEQ ID NOs: 1-9 are provided in the following Table 1.

In certain embodiments, the CDRs regions/sequences disclosed herein are delineated using the PyIgClassify system (Adolf-Bryfogle et al., Nucleic acids research 43.D1 (2015): D432-D438).

TABLE 1
anti-CD70 scFv

| CDRs | 1 | 2 | 3 |
|---|---|---|---|
| V_{H} | AASGFTFSSYIMH [SEQ ID NO: 1] | VISYDGRNKY [SEQ ID NO: 2] | ARDTDGYDFDY [SEQ ID NO: 3] |
| V_{L} | RASQSVSSYLA [SEQ ID NO: 4] | YDASNRAT [SEQ ID NO: 5] | QQRTNWPLT [SEQ ID NO: 6] |

| | |
|---|---|
| Full V_{H} | QVQLVESGGGVVQPGRSLRLSCAASGFTFSSYIMHWVRQAPGKGLEWVAVISYDGRNKYYADSVKGRFTISRDNSKNTLY |
| | LQMNSLRAEDTAVYYCARDTDGYDFDYWGQGBTLVTVSS [SEQ ID NO: 7] |
| Full V_{L} | EIVLTQSPATLSLSPGERATLSCRASQSVSSYLAWYQQKPGQAPRLLIYDASNRATGIPARFSGSGSGTDFTLTISSLEP |
| | EDFAVYYCQQRTNWPLTFGGGTKVEIK [SEQ ID NO: 8] |
| scFv | QVQLVESGGGVVQPGRSLRLSCAASGFTFSSYIMHWVRQAPGKGLEWVAVISYDGRNKYYADSVKGRFTISRDNSKNTLY |
| | LQMNSLRAEDTAVYYCARDTDGYDFDYWGQGTLVTVSSGGGGSGGGGSGGGGSEIVLTQSPATLSLSPGERATLSCRASQ |
| | SVSSYLAWYQQKPGQAPRLLIYDASNRATGIPARFSGSGSGTDFTLTISSLEPEDFAVYYCQQRTNWPLTFGGGTKVEIK |
| | [SEQ ID NO: 9] |

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 79. For example, the extracellular antigen-binding domain of the CAR comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 79. In certain embodiments, the extracellular antigen-binding domain of the CAR comprises a V_{H} comprising the amino acid sequence set forth in SEQ ID NO: 79. SEQ ID NO: 79 is provided below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 80. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 80. In certain embodiments, the extracellular antigen-binding domain of the CAR comprises a V_{L} comprising the amino acid sequence set forth in SEQ ID NO: 80. SEQ ID NO: 80 is provided below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 79, and a V_{L} comprising an amino acid sequence that is at least about 80% (e.g., at least about 85%, at least about 90%, or at least about 95%) homologous or identical to the amino acid sequence set forth in SEQ ID NO: 80.

In certain embodiments, the extracellular antigen-binding domain of the CAR comprises a V_{H} comprising the amino acid sequence set forth in SEQ ID NO: 79. In certain embodiments, the extracellular antigen-binding domain of the CAR comprises a V_{L} comprising the amino acid sequence set forth in SEQ ID NO: 80. In certain embodiments, the extracellular antigen-binding domain of the CAR comprises a V_{H} comprising the amino acid sequence set forth in SEQ ID NO: 79, and a V_{L} comprising the amino acid sequence set forth in SEQ ID NO: 80.

[SEQ ID NO: 79]
QVQLVESGGGVVQPGRSLRLSCAASGFTFSSYIMHWVRQAPGKGLE
WVAVISYDGRNKYYADSVKGRFTISRDNSKNTLYLQMNSLRAEDTA
VYYCARDTDGYDFDYWGQGTLVTV
[SEQ ID NO: 80]
EIVLTQSPATLSLSPGERATLSCRASQSVSSYLAWYQQKPGQAPRL
LIYDASNRATGIPARFSGSGSGTDFTLTISSLEPEDFAVYYCQQRT
NWPLTFGGGTKVEI

In certain embodiments, the V_{H} and V_{L} are linked via a linker. In certain embodiments, the linker comprises the amino acid sequence set forth in SEQ ID NO: 10.

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 7, 8, 79, and 80) may contain substitutions (e.g., conservative substitutions), insertions, or deletions relative to the specified sequence(s), but retain the ability to bind to a target antigen (e.g., CD70). In certain embodiments, a total of 1 to 10 amino acids are substituted, inserted and/or deleted in a specific sequence (e.g., SEQ ID NOs: 7, 8, 79, and 80). In certain embodiments, substitutions, insertions, or deletions occur in regions outside the CDRs (e.g., in the FRs) of the extracellular antigen-binding domain. In certain embodiments, the extracellular antigen-binding domain of the CAR comprises V_{H} and/or V_{L} sequence selected from SEQ ID NOs: 7, 8, 79, and 80, including post-translational modifications of that sequence (SEQ ID NO: 7, 8, 79, and 80). In certain embodiments, the first antigen-recognizing receptor is a CAR that comprises a transmembrane domain. Different transmembrane domains result in different receptor stability. After antigen recognition, receptors cluster and a signal are transmitted to the cell. In accordance with the presently disclosed subject matter, the transmembrane domain of the first antigen-recognizing receptor can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain of the CAR comprises a CD28 polypeptide (e.g., the transmembrane domain of CD28 or a portion thereof). In certain embodiments, the transmembrane domain of the CAR comprises a transmembrane domain of human CD28 or a portion thereof. In certain embodiments, the CD28 polypeptide comprises or consists of an amino acid sequence that is a consecutive portion of the amino acid sequence having a NCBI Reference No: NP_006130 (SEQ ID NO: 11), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 25. In certain embodiments, the transmembrane domain of the CAR comprises a CD28 polypeptide that comprises or consists of amino acids 153 to 179 of SEQ ID NO: 11. SEQ ID NO: 11 is provided below.

[SEQ ID NO: 11]
MLRLLLALNL FPSIQVTGNK ILVKQSPMLV AYDNAVNLSC
KYSYNLESRE FRASLHKGLD SAVEVCVVYG NYSQQLQVYS
KTGENCDGKL GNESVTFYLQ NLYVNQTDIY FCKIEVMYPP
PYLDNEKSNG TIIHVKGKHL CPSPLFPGPS KPFWVLVVVG
GVLACYSLLV TVAFIIFWVR SKRSRLLHSD YMNMTPRRPG
PTRKHYQPYA PPRDFAAYRS

In certain embodiments, the first antigen-recognizing receptor is a CAR that further comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 11), a portion of a CD8 polypeptide, or a synthetic spacer sequence.

In certain embodiments, the first antigen-recognizing receptor is a CAR that further comprises a hinge/spacer region comprising a native or modified hinge region of a CD28 polypeptide. In certain embodiments, the hinge/spacer region of the first antigen-recognizing receptor (e.g., a CAR) comprises a CD28 polypeptide comprising or consisting of amino acids 114 to 152 of SEQ ID NO: 11.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8a polypeptide and the transmembrane domain comprises a CD8a polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

In certain embodiments, the first antigen-recognizing receptor is a CAR that comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T-cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the native CD3ζ comprises or consists of an amino acid sequence that is at least about 85%, about 90%, about 95%, about 96%, about 97%, about 98%, about 99% or about 100% identical or homologous to the amino acid sequence having a NCBI Reference No: NP_932170 (SEQ ID NO: 12) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 12, which is at least about 20, or at least about 30, or at least about 40, or at least about 50, and up to about 164 amino acids in length. In certain embodiments, the native CD3ζ comprises or consists of the amino acid sequence of amino acids 1 to 164, 1 to 50, 50 to 100, 52 to 164, 100 to 150, or 150 to 164 of SEQ ID NO: 12. In certain embodiments, the intracellular signaling domain of the CAR comprises a native CD3ζ comprising or consisting of the amino acid sequence of amino acids 52 to 164 of SEQ ID NO: 12. SEQ ID NO: 12 is provided below:

[SEQ ID NO: 12]
MKWKALFTAA ILQAQLPITE AQSFGLLDPK LCYLLDGILF
IYGVILTALF LRVKFSRSAD APAYQQGQNQ LYNELNLGRR
EEYDVLDKRR GRDPEMGGKP QRRKNPQEGL YNELQKDKMA
EAYSEIGMKG ERRRGKGHDG LYQGLSTATK DTYDALHMQA
LPPR

In certain embodiments, the native CD3ζ comprises or consists of an amino acid sequence that is at least about 85%, about 90%, about 95%, about 96%, about 97%, about 98%, about 99% or about 100% identical or homologous to the amino acid sequence set forth in SEQ ID NO: 13. SEQ ID NO: 13 is provided below:

[SEQ ID NO: 13]
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 14.

[SEQ ID NO: 14]
QNQLYNELNLGRREEYDVLDKR

An exemplary nucleic acid sequence encoding the amino acid sequence of SEQ ID NO: 14 is set forth in SEQ ID NO: 15, which is provided below.

[SEQ ID NO: 15]
CAGAACCAGCTCTATAACGAGCTCAATCTAGGACGAAGAGAGGAGTACG
ATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 16, which is provided below.

[SEQ ID NO: 16]
QNQLFNELNLGRREEFDVLDKR

An exemplary nucleic acid sequence encoding the amino acid sequence of SEQ ID NO: 16 is set forth in SEQ ID NO: 17, which is provided below.

[SEQ ID NO: 17]
CAGAACCAGCTCTTTAACGAGCTCAATCTAGGACGAAGAGAGGAGTTCG
ATGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 18, which is provided below.

[SEQ ID NO: 18]
QEGLYNELQKDKMAEAYSEIGMK

An exemplary nucleic acid sequence encoding the amino acid sequence of SEQ ID NO: 18 is set forth in SEQ ID NO: 19, which is provided below.

[SEQ ID NO: 19]
CAGGAAGGCCTGTACAATGAACTGCAGAAAGATAAGATGGCGGAGGCCT
ACAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 20, which is provided below.

[SEQ ID NO: 20]
QEGLFNELQKDKMAEAFSEIGMK

An exemplary nucleic acid sequence encoding the amino acid sequence of SEQ ID NO: 20 is set forth in SEQ ID NO: 21, which is provided below.

[SEQ ID NO: 21]
CAGGAAGGCCTGTTCAATGAACTGCAGAAAGATAAGATGGCGGAGGCCT
TCAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 22, which is provided below.

[SEQ ID NO: 22]
HDGLYQGLSTATKDTYDALHMQ

An exemplary nucleic acid sequence encoding the amino acid sequence of SEQ ID NO: 22 is set forth in SEQ ID NO: 23, which is provided below.

[SEQ ID NO: 23]
CACGATGGCCTTTACCAGGGTCTCAGTACAGCCACCAAGGACACCTACG
ACGCCCTTCACATGCAG

In certain embodiments, the modified CD3ζ polypeptide comprises an ITAM3 variant. In certain embodiments, the ITAM3 variant comprises or consists of two loss-of-function mutations.

In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 24, which is provided below.

[SEQ ID NO: 24]
HDGLFQGLSTATKDTFDALHMQ

An exemplary nucleic acid sequence encoding the amino acid sequence of SEQ ID NO: 24 is set forth in SEQ ID NO: 25, which is provided below.

[SEQ ID NO: 25]
CACGATGGCCTTTTCCAGGGGCTCAGTACAGCCACCAAGGACACCTTCG
ACGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 14, an ITAM2 variant consisting of the amino acid sequence set forth in SEQ ID NO: 20, and an ITAM3 variant consisting of the amino acid sequence set forth in SEQ ID NO: 24. In certain embodiments, the CAR is designated as “1XX”. In certain embodiments, the modified CD3ζ polypeptide comprises or consists of the amino acid sequence set forth in SEQ ID NO: 26. SEQ ID NO: 26 is provided below:

[SEQ ID NO: 26]
RVKFSRSADA PAYQQGQNQL YNELNLGRRE EYDVLDKRRG
RDPEMGGKPR RKNPQEGLEN ELQKDKMAEA FSEIGMKGER
RRGKGHDGLF QGLSTATKDT FDALHMQALP PR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 26 or a fragment thereof, and/or may optionally comprise up to one or up to two or up to three conservative amino acid substitutions.

An exemplary nucleic acid sequence encoding the amino acid sequence of SEQ ID NO: 26 is set forth in SEQ ID NO: 27, which is provided below.

[SEQ ID NO: 27]
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTTCAATGAACTGCAGAAAGATA
AGATGGCGGAGGCCTTCAGTGAGATTGGGATGAAAGGCGAGCGCCGGAG
GGGCAAGGGGCACGATGGCCTTTTCCAGGGGCTCAGTACAGCCACCAAG
GACACCTTCGACGCCCTTCACATGCAGGCCCTGCCCCCTCGC

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, CD150, CD226, and NKG2D.

In certain embodiments, the intracellular signaling domain of the CAR comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the first antigen-recognizing receptor comprise or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 11 or a fragment thereof, and/or may optionally comprise up to one or up to two or up to three conservative amino acid substitutions. In certain embodiments, the CD28 polypeptide comprised in the co-stimulatory signaling region of the CAR comprises or consist of an amino acid sequence that is a consecutive portion of SEQ ID NO: 11, which is at least about 20, or at least about 30, or at least about 40, or at least about 50, and up to about 220 amino acids in length. Alternatively or additionally, in certain embodiments, the CD28 polypeptide comprised in the co-stimulatory signaling region of the CAR comprises or consists of amino acids 1 to 220, 1 to 50, 50 to 100, 100 to 150, 114 to 220, 150 to 200, 180 to 220, or 200 to 220 of SEQ ID NO: 11. In certain embodiments, the intracellular signaling domain of the CAR comprises a co-stimulatory signaling region that comprises a CD28 polypeptide comprising or consisting of amino acids 180 to 220 of SEQ ID NO: 11.

An exemplary nucleic acid sequence encoding the amino acid sequence of amino acids 180 to 220 of SEQ ID NO: 11 is set forth in SEQ ID NO: 28, which is provided below.

[SEQ ID NO: 28]
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC

In certain embodiments, the intracellular signaling domain of the first antigen-recognizing receptor comprises a co-stimulatory signaling region that comprises an intracellular domain of mouse CD28 or a portion thereof. In certain embodiments, the CD28 polypeptide comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having a NCBI Reference No: NP_031668.3 (or SEQ ID NO: 29) or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 29, which is at least about 20, or at least about 30, or at least about 40, or at least about 50, and up to 218 amino acids in length. In certain embodiments, the CD28 polypeptide comprised in the co-stimulatory signaling region of the CAR comprises or consists of the amino acid sequence of amino acids 1 to 218, 1 to 50, 50 to 100, 100 to 150, 150 to 218, 178 to 218, or 200 to 218 of SEQ ID NO: 29. In certain embodiments, the co-stimulatory signaling region of the CAR comprises a CD28 polypeptide that comprises or consists of amino acids 178 to 218 of SEQ ID NO: 29. SEQ ID NO: 29 is provided below.

[SEQ ID NO: 29]
MTLRLLFLAL NFFSVQVTEN KILVKQSPLL VVDSNEVSLS
CRYSYNLLAK EFRASLYKGV NSDVEVCVGN GNFTYQPQFR
SNAEENCDGD FDNETVTERL WNLHVNHTDI YFCKIEFMYP
PPYLDNERSN GTIIHIKEKH LCHTQSSPKL FWALVVVAGV
LFCYGLLVTV ALCVIWTNSR RNRLLQSDYM NMTPRRPGLT
RKPYQPYAPA RDFAAYRP

In certain embodiments, the intracellular signaling domain of the CAR comprises a co-stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region of the CAR comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having a NCBI Ref. No.: NP_001552 (SEQ ID NO: 30) or a fragment thereof, and/or may optionally comprise up to one or up to two or up to three conservative amino acid substitutions. In certain embodiments, the 4-1BB comprised in the co-stimulatory signaling region of the CAR comprises or consists of an amino acid sequence that is a consecutive portion of SEQ ID NO: 30, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the co-stimulatory signaling region of the CAR comprises a 4-1BB polypeptide that comprises or consists of the amino acid sequence of amino acids 1 to 255, 1 to 50, 50 to 100, 100 to 150, 150 to 200, or 200 to 255 of SEQ ID NO: 30. In certain embodiments, the co-stimulatory signaling region of the CAR comprises a 4-1BB polypeptide comprising or consisting of the amino acid sequence of amino acids 214 to 255 of SEQ ID NO: 30. SEQ ID NO: 30 is provided below.

[SEQ ID NO: 30]
MGNSCYNIVA TLLLVLNFER TRSLQDPCSN CPAGTFCDNN
RNQICSPCPP NSESSAGGQR TCDICRQCKG VFRTRKECSS
TSNAECDCTP GFHCLGAGCS MCEQDCKQGQ ELTKKGCKDC
CFGTENDQKR GICRPWTNCS LDGKSVLVNG TKERDVVCGP
SPADLSPGAS SVTPPAPARE PGHSPQIISF FLALTSTALL
FLLFFLTLRF SVVKRGRKKL LYIFKQPFMR PVQTTQEEDG
CSCRFPEEEE GGCEL

In certain embodiments, the intracellular signaling domain of the CAR comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, CD150, CD226, and NKG2D. In certain embodiments, the intracellular signaling domain of the CAR comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

In certain embodiments, the first antigen-recognizing receptor is a CAR that comprises i) an extracellular antigen-binding domain that binds to CD70 (e.g., human CD70), ii) a transmembrane domain comprising a CD28 polypeptide (e.g., human CD28 polypeptide, e.g., a transmembrane domain of CD28 (e.g., human CD28) or a portion thereof), iii) a hinge/spacer region derived from a CD28 polypeptide (e.g., a human CD28 polypeptide), iv) an intracellular signaling domain comprising a) a modified CD3ζ polypeptide (e.g., a modified human CD3ζ polypeptide) comprising a native ITAM1, an ITAM2 variant consisting of two loss-of-function mutations, and an ITAM3 variant consisting of two loss-of-function mutations, and b) a co-stimulatory signaling region comprising a CD28 polypeptide (e.g., a human CD28 polypeptide, e.g., an intracellular domain of CD28 (e.g., human CD28) of a portion thereof). In certain embodiments, the extracellular antigen-binding domain comprises i) a heavy chain variable region that comprises a V_{H} comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region (V_{L}) comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6. In certain embodiments, the V_{H} comprises the amino acid sequence set forth in SEQ ID NO: 7. In certain embodiments, the V_{L} comprises the amino acid sequence set forth in SEQ ID NO: 8. In certain embodiments, the extracellular antigen-binding domain comprises a linker between the V_{H} and the V_{L}. In certain embodiments, the linker comprises or consists of the amino acid sequence set forth in SEQ ID NO: 10. In certain embodiments, the transmembrane domain comprises a CD28 polypeptide comprising or consisting of amino acids 153 to 179 of SEQ ID NO: 11. In certain embodiments, the hinge/spacer region comprises a CD28 polypeptide comprising or consisting of amino acids 114 to 152 of SEQ ID NO: 11. In certain embodiments, the intracellular signaling domain comprises a modified CD3ζ polypeptide comprising or consisting of the amino acid sequence set forth in SEQ ID NO: 26, and a co-stimulatory signaling region comprising a CD28 polypeptide that comprises or consists of 180 to 220 of SEQ ID NO: 11. In certain embodiments, the CAR is designated as “CD70-28z1XX”. In certain embodiments, the CAR (e.g., CD70-28z1XX) comprises an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 31. In certain embodiments, the CAR (e.g., CD70-28z1XX) comprises the amino acid sequence set forth in SEQ ID NO: 31. SEQ ID NO: 31 is provided below.

[SEQ ID NO: 31]
QVQLVESGGGVVQPGRSLRLSCAASGFTFSSYIMHWVRQAPGKGLEWVA
VISYDGRNKYYADSVKGRFTISRDNSKNTLYLQMNSLRAEDTAVYYCAR
DTDGYDFDYWGQGTLVTVSSGGGGSGGGGSGGGGSEIVLTQSPATLSLS
PGERATLSCRASQSVSSYLAWYQQKPGQAPRLLIYDASNRATGIPARFS
GSGSGTDFTLTISSLEPEDFAVYYCQQRTNWPLTFGGGTKVEIKAAAIE
VMYPPPYLDNEKSNGTIIHVKGKHLCPSPLFPGPSKPFWVLVVVGGVLA
CYSLLVTVAFIIFWVRSKRSRLLHSDYMNMTPRRPGPTRKHYQPYAPPR
DFAAYRSRVKFSRSADAPAYQQGQNQLYNELNLGRREEYDVLDKRRGRD
PEMGGKPRRKNPQEGLFNELQKDKMAEAFSEIGMKGERRRGKGHDGLFQ
GLSTATKDTFDALHMQALPPR

An exemplary nucleotide sequence encoding the amino acid sequence of SEQ ID NO: 31 is set forth in SEQ ID NO: 32, which is provided below.

[SEQ ID NO: 32]
CAGGTGCAGCTGGTGGAGTCTGGGGGAGGCGTGGTCCAGCCTGGGAGGT
CCCTGAGACTCTCCTGTGCAGCCTCTGGATTTACCTTCAGTAGCTATAT
TATGCACTGGGTCCGCCAGGCTCCAGGCAAGGGGCTGGAGTGGGTGGCA
GTTATATCATATGATGGAAGAAACAAATACTACGCAGACTCCGTGAAGG
GCCGATTCACCATCTCCAGAGACAATTCCAAGAACACGCTGTATCTGCA
AATGAACAGCCTGAGAGCTGAGGACACGGCTGTGTATTACTGTGCGAGA
GATACGGATGGCTACGATTTTGACTACTGGGGCCAGGGAACCCTGGTCA
CCGTCTCCTCAGGTGGAGGTGGATCAGGTGGAGGTGGATCTGGTGGAGG
TGGATCTGAAATTGTGTTGACACAGTCTCCAGCCACCCTGTCTTTGTCT
CCAGGGGAAAGAGCCACCCTCTCCTGCAGGGCCAGTCAGAGTGTTAGCA
GCTACTTAGCCTGGTACCAACAGAAACCTGGCCAGGCTCCCAGGCTCCT
CATCTATGATGCATCCAACAGGGCCACTGGCATCCCAGCCAGGTTCAGT
GGCAGTGGGTCTGGGACAGACTTCACTCTCACCATCAGCAGCCTAGAGC
CTGAAGATTTTGCAGTTTATTACTGTCAGCAGCGTACCAACTGGCCGCT
CACTTTCGGCGGAGGGACCAAGGTGGAGATCAAAGCGGCCGCAATTGAA
GTTATGTATCCTCCTCCTTACCTAGACAATGAGAAGAGCAATGGAACCA
TTATCCATGTGAAAGGGAAACACCTTTGTCCAAGTCCCCTATTTCCCGG
ACCTTCTAAGCCCTTTTGGGTGCTGGTGGTGGTTGGTGGAGTCCTGGCT
TGCTATAGCTTGCTAGTAACAGTGGCCTTTATTATTTTCTGGGTGAGGA
GTAAGAGGAGCAGGCTCCTGCACAGTGACTACATGAACATGACTCCCCG
CCGCCCCGGGCCCACCCGCAAGCATTACCAGCCCTATGCCCCACCACGC
GACTTCGCAGCCTATCGCTCCAGAGTGAAGTTCAGCAGGAGCGCAGACG
CCCCCGCGTACCAGCAGGGCCAGAACCAGCTCTATAACGAGCTCAATCT
AGGACGAAGAGAGGAGTACGATGTTTTGGACAAGAGACGTGGCCGGGAC
CCTGAGATGGGGGGAAAGCCGAGAAGGAAGAACCCTCAGGAAGGCCTGT
TCAATGAACTGCAGAAAGATAAGATGGCGGAGGCCTTCAGTGAGATTGG
GATGAAAGGCGAGCGCCGGAGGGGCAAGGGGCACGATGGCCTTTTCCAG
GGGCTCAGTACAGCCACCAAGGACACCTTCGACGCCCTTCACATGCAGG
CCCTGCCCCCTCGC

In addition, the extracellular antigen-binding domain of the CAR can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the CAR. Exemplary leader sequences include, but is not limited to, a human IL-2 signal sequence (e.g., a human TL-2 signal sequence comprising or consisting of the amino acid sequence set forth in SEQ ID NO: 33), a mouse IL-2 signal sequence (e.g., a mouse IL-2 signal sequence comprising or consisting of the amino acid sequence set forth in SEQ ID NO: 34); a human kappa leader sequence (e.g., a human kappa leader sequence comprising or consisting of the amino acid sequence set forth in SEQ ID NO: 35), a mouse kappa leader sequence (e.g., a mouse kappa leader sequence comprising or consisting of the amino acid sequence set forth in SEQ ID NO: 36); a human CD8 leader sequence (e.g., a human CD8 leader sequence comprising or consisting of the amino acid sequence set forth in SEQ ID NO: 37); a truncated human CD8 signal peptide (e.g., a truncated human CD8 signal peptide comprising or consisting of the amino acid sequence set forth in SEQ ID NO: 38); a human albumin signal sequence (e.g., a human albumin signal sequence comprising or consisting of the amino acid sequence set forth in SEQ ID NO: 39); and a human prolactin signal sequence (e.g., a human prolactin signal sequence comprising or consisting of the amino acid sequence set forth in SEQ ID NO: 40). SEQ ID Nos: 33-40 are provided below.

[SEQ ID NO: 33]
MYRMQLLSCIALSLALVINS
[SEQ ID NO: 34]
MYSMQLASCVTLTLVLLVNS
[SEQ ID NO: 35]
METPAQLLFLLLLWLPDTTG
[SEQ ID NO: 36]
METDTLLLWVLLLWVPGSTG
[SEQ ID NO: 37]
MALPVTALLLPLALLLHAARP
[SEQ ID NO: 38]
MALPVTALLLPLALLLHA
[SEQ ID NO: 39]
MKWVTFISLLESSAYS
[SEQ ID NO: 40]
MDSKGSSQKGSRLLLLLVVSNLLLCQGVVS

In certain embodiments, the signal peptide comprises a CD8 polypeptide, e.g., the CAR comprises a truncated CD8 signal peptide. In certain embodiments, the signal peptide comprises or consists of the amino acid sequence set forth in SEQ ID NO: 38.

5.3.1.2.2 Chimeric Ligand Receptors

In certain embodiments, the first antigen-recognizing receptor is a chimeric ligand receptor that comprises a ligand or a portion thereof that binds to the first antigen. In certain embodiments, the first antigen is CD70. In certain embodiments, the ligand is CD27. In certain embodiments, the chimeric ligand receptor further comprises a transmembrane domain and an intracellular signaling domain.

In certain embodiments, the transmembrane domain is fused to the ligand or portion thereof. In certain embodiments, the transmembrane domain is fused to the intracellular signaling domain. In certain embodiments, the transmembrane domain is positioned between the ligand or portion thereof and the intracellular signaling domain. In certain embodiments the transmembrane domain of the chimeric ligand receptor is a transmembrane domain disclosed in Section 5.3.1.2.1. In certain embodiments, the intracellular signaling domain of the chimeric ligand receptor comprises a CD3ζ polypeptide (e.g., as disclosed in Section 5.3.1.2.1).

Additional information on the presently disclosed chimeric ligand receptor can be found in Sauer et al., Blood (2021) 138 (4): 318-330, the content of which is incorporated by reference in its entirety.

5.3.1.3. T Cell Receptors (TCRs)

In certain embodiments, the first antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the first antigen-recognizing receptor is an endogenous TCR. In certain embodiments, the first antigen-recognizing receptor is naturally occurring TCR.

In certain embodiments, the first antigen-recognizing receptor is an exogenous TCR. In certain embodiments, the first antigen-recognizing receptor is a recombinant TCR. In certain embodiments, the first antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

5.3.1.4. TCR Like Fusion Molecules

In certain embodiments, the first antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule is a recombinant T cell receptor (TCR). In certain embodiments, the recombinant TCR comprises at least one antigen-binding chain. In certain embodiments, the antigen-binding domain of the recombinant TCR comprises a ligand for a cell-surface receptor, a receptor for a cell surface ligand, an antigen binding portion of an antibody or a fragment thereof, or an antigen binding portion of a TCR. In certain embodiments, the recombinant TCR comprises two antigen binding chains, i.e., a first antigen binding chain and a second antigen binding chain. In certain embodiments, the first and second antigen-binding chains each comprises a constant domain. In certain embodiments, the recombinant TCR binds to an antigen (e.g., a first antigen or a second antigen) in an HLA-independent manner. Thus, in certain embodiments, the recombinant TCR is an HLA-independent (or non-HLA restricted) TCR (referred to as “HIT-CAR” or “HIT”).

In certain embodiments, the first antigen-binding chain comprises an antigen-binding fragment of a heavy chain variable region (V_{H}) of an antibody. In certain embodiments, the second antigen-binding chain comprises an antigen-binding fragment of a light chain variable region (V_{L}) of an antibody. In certain embodiments, the first antigen-binding chain comprises an antigen-binding fragment of a V_{H} of an antibody, and the second antigen-binding chain comprises an antigen-binding fragment of a V_{L} of the antibody.

In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the first antigen-binding chain comprises a TRAC polypeptide, and the second antigen-binding chain comprises a TRBC polypeptide. In certain embodiments, the first antigen-binding chain comprises a TRBC polypeptide, and the second antigen-binding chain comprises a TRAC polypeptide.

In certain embodiments, the first antigen-binding chain comprises a V_{H} of an antibody and a TRAC polypeptide, and the second antigen-binding chain comprises a V_{L} of an antibody and a TRBC polypeptide.

In certain embodiments, the first antigen-binding chain comprises a V_{H} of an antibody and a TRBC polypeptide, and the second antigen-binding chain comprises a V_{L} of an antibody and a TRAC polypeptide.

In certain embodiments, at least one of the TRAC polypeptide and the TRBC polypeptide is endogenous. In certain embodiments, the TRAC polypeptide is endogenous. In certain embodiments, the TRBC polypeptide is endogenous. In certain embodiments, both the TRAC polypeptide and the TRBC polypeptide are endogenous.

In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex.

In certain embodiments, the first antigen binding chain and the second antigen binding chain bind to CD70 (e.g., human CD70).

In certain embodiments, the first antigen binding chain comprises a heavy chain variable region (V_{H}) comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. In certain embodiments, the first antigen binding chain comprises a V_{H} comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3.

In certain embodiments, the second antigen binding chain comprises a light chain variable region (V_{L}) comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. In certain embodiments, the second antigen binding chain comprises a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the first antigen binding chain comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3, a conservative modification thereof, and the second antigen binding chain comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. In certain embodiments, the first antigen binding chain comprises a V_{H} comprising a CDR1 comprising amino acids having the sequence set forth in SEQ ID NO: 1, a CDR2 comprising the amino acid sequence set forth in SEQ ID NO: 2, and a CDR3 comprising the amino acid sequence set forth in SEQ ID NO: 3; and the second antigen binding chain comprises a V_{L} comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the first antigen binding chain comprises a CDR1, a CDR2, and a CDR3 of a V_{H} sequence of an anti-CD70 antibody disclosed in International Patent Publication No. WO 2007/038637. In certain embodiments, the first antigen binding chain comprises a CDR1, a CDR2, and a CDR3 of the V_{H} sequence of the anti-CD70 antibody 2H5 disclosed in International Patent Publication No. WO 2007/038637.

In certain embodiments, the second antigen binding chain comprises a CDR1, a CDR2, and a CDR3 of a V_{L} sequence of an anti-CD70 antibody disclosed in International Patent Publication No. WO 2007/038637. In certain embodiments, the second antigen binding chain comprises a CDR1, a CDR2, and a CDR3 of the V_{L} sequence of the anti-CD70 antibody 2H5 disclosed in International Patent Publication No. WO 2007/038637.

In certain embodiments, the first antigen binding chain comprises a V_{H} sequence of an anti-CD70 antibody disclosed in International Patent Publication No. WO 2007/038637. In certain embodiments, the second antigen binding chain comprises a V_{L} sequence of the anti-CD70 antibody 2H5 disclosed in International Patent Publication No. WO 2007/038637.

In certain embodiments, the first antigen binding chain comprises a V_{H} comprising an amino acid sequence that is at least about 80% (e.g., at least about 85%, at least about 90%, or at least about 95%) homologous or identical to the amino acid sequence set forth in SEQ ID NO: 7. For example, the first antigen binding chain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 7. In certain embodiments, the first antigen binding chain comprises a V_{H} comprising the amino acid sequence set forth in SEQ ID NO: 7.

In certain embodiments, the second antigen binding chain comprises a V_{L} comprising an amino acid sequence that is at least about 80% (e.g., at least about 85%, at least about 90%, or at least about 95%) homologous or identical to the amino acid sequence set forth in SEQ ID NO: 8. For example, the second antigen binding chain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 8. In certain embodiments, the second antigen binding chain comprises a V_{L} comprising the amino acid sequence set forth in SEQ ID NO: 8.

In certain embodiments, the first antigen binding chain comprises a V_{H} comprising an amino acid sequence that is at least about 80% (e.g., at least about 85%, at least about 90%, or at least about 95%) homologous or identical to the amino acid sequence set forth in SEQ ID NO: 7, and the second antigen binding chain comprises a V_{L} comprising an amino acid sequence that is at least about 80% (e.g., at least about 85%, at least about 90%, or at least about 95%) homologous or identical to the amino acid sequence set forth in SEQ ID NO: 8.

In certain embodiments, the first antigen binding chain comprises a V_{H} comprising the amino acid sequence set forth in SEQ ID NO: 7. In certain embodiments, the second antigen binding chain comprises a V_{L} comprising the amino acid sequence set forth in SEQ ID NO: 8. In certain embodiments, the first antigen binding chain comprises a V_{H} comprising the amino acid sequence set forth in SEQ ID NO: 7, and the second antigen binding chain comprises a V_{L} comprising the amino acid sequence set forth in SEQ ID NO: 8.

In certain embodiments, the first antigen binding chain comprises a V_{H} comprising an amino acid sequence that is at least about 80% (e.g., at least about 85%, at least about 90%, or at least about 95%) homologous or identical to the amino acid sequence set forth in SEQ ID NO: 79. For example, the first antigen binding chain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 79. In certain embodiments, the first antigen binding chain comprises a V_{H} comprising the amino acid sequence set forth in SEQ ID NO: 79.

In certain embodiments, the second antigen binding chain comprises a V_{L} comprising an amino acid sequence that is at least about 80% (e.g., at least about 85%, at least about 90%, or at least about 95%) homologous or identical to the amino acid sequence set forth in SEQ ID NO: 80. For example, the second antigen binding chain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 80. In certain embodiments, the second antigen binding chain comprises a V_{L} comprising the amino acid sequence set forth in SEQ ID NO: 80.

In certain embodiments, the first antigen binding chain comprises a V_{H} comprising an amino acid sequence that is at least about 80% (e.g., at least about 85%, at least about 90%, or at least about 95%) homologous or identical to the amino acid sequence set forth in SEQ ID NO: 79, and the second antigen binding chain comprises a V_{L} comprising an amino acid sequence that is at least about 80% (e.g., at least about 85%, at least about 90%, or at least about 95%) homologous or identical to the amino acid sequence set forth in SEQ ID NO: 80.

In certain embodiments, the first antigen binding chain comprises a V_{H} comprising the amino acid sequence set forth in SEQ ID NO: 79. In certain embodiments, the second antigen binding chain comprises a V_{L} comprising the amino acid sequence set forth in SEQ ID NO: 80. In certain embodiments, the first antigen binding chain comprises a V_{H} comprising the amino acid sequence set forth in SEQ ID NO: 79, and the second antigen binding chain comprises a V_{L} comprising the amino acid sequence set forth in SEQ ID NO: 80.

In certain embodiments, the first and second antigen binding chains bind to an antigen with a dissociation constant (K_{D}) of about 2×10⁻⁷ M or less. In certain embodiments, the first and second antigen binding chains bind to an antigen with a high binding affinity. In certain embodiments, the K_{D} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less. In certain embodiments, the K_{D} is about 1×10⁻⁸ M or less. In certain embodiments, the K_{D} is about 3×10⁻⁹ M or less. In certain embodiments, the K_{D} is about 5×10⁻⁹ M or less. In certain embodiments, the K_{D} is from about 1×10⁻⁹ M to about 1×10⁻⁸ M. In certain embodiments, the K_{D} is from about 1.5×10⁻⁹ M to about 1×10⁻⁸ M. In certain embodiments, the K_{D} is from about 5×10⁻⁹ M to about 1×10⁻⁸ M.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the first antigen binding chain or the second antigen binding chain comprises a constant domain that comprises a native or modified TRAC polypeptide. In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO:41 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 41. SEQ ID NO:41 is provided below.

[SEQ ID NO: 41]
IQNPDPAVYQLRDSKSSDKSVCLFTDFDSQTNVSQSKDSDVYITDKTVL
DMRSMDFKSNSAVAWSNKSDFACANAENNSIIPEDTFFPSPESSCDVKL
VEKSFETDTNLNFQNLSVIGFRILLLKVAGENLLMTLRLWSS

An exemplary nucleotide sequence encoding the amino acid sequence of SEQ ID NO: 41 is set forth in SEQ ID NO: 69, which is provided below.

[SEQ ID NO: 69]
ATCCAGAACCCTGACCCTGCCGTGTACCAGCTGAGAGACTCTAAATCCA
GTGACAAGTCTGTCTGCCTATTCACCGATTTTGATTCTCAAACAAATGT
GTCACAAAGTAAGGATTCTGATGTGTATATCACAGACAAAACTGTGCTA
GACATGAGGTCTATGGACTTCAAGAGCAACAGTGCTGTGGCCTGGAGCA
ACAAATCTGACTTTGCATGTGCAAACGCCTTCAACAACAGCATTATTCC
AGAAGACACCTTCTTCCCCAGCCCAGAAAGTTCCTGTGATGTCAAGCTG
GTCGAGAAAAGCTTTGAAACAGATACGAACCTAAACTTTCAAAACCTGT
CAGTGATTGGGTTCCGAATCCTCCTCCTGAAAGTGGCCGGGTTTAATCT
GCTCATGACGCTGCGGCTGTGGTCCAGC

In certain embodiments, the TRAC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by the gene of NCBI Genbank ID: 28755, NG_001332.3, range 925603 to 930229 (SEQ ID NO:42) or a fragment thereof, and/or may optionally comprise up to one or up to two or up to three conservative amino acid substitutions. In certain embodiments, the TRAC polypeptide comprises or consists of the amino acid sequence encoded by the nucleotide sequence of SEQ ID NO: 42. SEQ ID NO: 42 is provided below.

| [SEQ ID NO: 42] | |
|---|---|
| 1 | atatccagaa ccctgaccct gccgtgtacc agctgagaga ctctaaatcc agtgacaagt |
| 61 | ctgtctgcct attcaccgat tttgattctc aaacaaatgt gtcacaaagt aaggattctg |
| 121 | atgtgtatat cacagacaaa actgtgctag acatgaggtc tatggacttc aagagcaaca |
| 181 | gtgctgtggc ctggagcaac aaatctgact ttgcatgtgc aaacgccttc aacaacagca |
| 241 | ttattccaga agacaccttc ttccccagcc caggtaaggg cagctttggt gccttcgcag |
| 301 | gctgtttcct tgcttcagga atggccaggt tctgcccaga gctctggtca atgatgtcta |
| 361 | aaactcctct gattggtggt ctcggcctta tccattgcca ccaaaaccct ctttttacta |
| 421 | agaaacagtg agccttgttc tggcagtcca gagaatgaca cgggaaaaaa gcagatgaag |
| 481 | agaaggtggc aggagagggc acgtggccca gcctcagtct ctccaactga gttcctgcct |
| 541 | gcctgccttt gctcagactg tttgcccctt actgctcttc taggcctcat tctaagcccc |
| 601 | ttctccaagt tgcctctcct tatttctccc tgtctgccaa aaaatctttc ccagctcact |
| 661 | aagtcagtct cacgcagtca ctcattaacc caccaatcac tgattgtgcc ggcacatgaa |
| 721 | tgcaccaggt gttgaagtgg aggaattaaa aagtcagatg aggggtgtgc ccagaggaag |
| 781 | caccattcta gttgggggag cccatctgtc agctgggaaa agtccaaata acttcagatt |
| 841 | ggaatgtgtt ttaactcagg gttgagaaaa cagctacctt caggacaaaa gtcagggaag |
| 901 | ggctctctga agaaatgcta cttgaagata ccagccctac caagggcagg gagaggaccc |
| 961 | tatagaggcc tgggacagga gctcaatgag aaaggagaag agcagcaggc atgagttgaa |
| 1021 | tgaaggaggc agggccgggt cacagggcct tctaggccat gagagggtag acagtattct |
| 1081 | aaggacgcca gaaagctgtt gatcggcttc aagcagggga gggacaccta atttgctttt |
| 1141 | cttttttttt tttttttttt tttttttttt tgagatggag ttttgctctt gttgcccagg |
| 1201 | ctggagtgca atggtgcatc ttggctcact gcaacctccg cctcccaggt tcaagtgatt |
| 1261 | ctcctgcctc agcctcccga gtagctgaga ttacaggcac ccgccaccat gcctggctaa |
| 1321 | ttttttgtat ttttagtaga gacagggttt cactatgttg gccaggctgg tctcgaactc |
| 1381 | ctgacctcag gtgatccacc cgcttcagcc tcccaaagtg ctgggattac aggcgtgagc |
| 1441 | caccacaccc ggcctgcttt tcttaaagat caatctgagt gctgtacgga gagtgggttg |
| 1501 | taagccaaga gtagaagcag aaagggagca gttgcagcag agagatgatg gaggcctggg |
| 1561 | cagggtggtg gcagggaggt aaccaacacc attcaggttt caaaggtaga accatgcagg |
| 1621 | gatgagaaag caaagagggg atcaaggaag gcagctggat tttggcctga gcagctgagt |
| 1681 | caatgatagt gccgtttact aagaagaaac caaggaaaaa atttggggtg cagggatcaa |
| 1741 | aactttttgg aacatatgaa agtacgtgtt tatactcttt atggcccttg tcactatgta |
| 1801 | tgcctcgctg cctccattgg actctagaat gaagccaggc aagagcaggg tctatgtgtg |
| 1861 | atggcacatg tggccagggt catgcaacat gtactttgta caaacagtgt atattgagta |
| 1921 | aatagaaatg gtgtccagga gccgaggtat cggtcctgcc agggccaggg gctctcccta |
| 1981 | gcaggtgctc atatgctgta agttccctcc agatctctcc acaaggaggc atggaaaggc |
| 2041 | tgtagttgtt cacctgccca agaactagga ggtctggggt gggagagtca gcctgctctg |
| 2101 | gatgctgaaa gaatgtctgt ttttcctttt agaaagttcc tgtgatgtca agctggtcga |
| 2161 | gaaaagcttt gaaacaggta agacaggggt ctagcctggg tttgcacagg attgcggaag |
| 2221 | tgatgaaccc gcaataaccc tgcctggatg agggagtggg aagaaattag tagatgtggg |
| 2281 | aatgaatgat gaggaatgga aacagcggtt caagacctgc ccagagctgg gtggggtctc |
| 2341 | tcctgaatcc ctctcaccat ctctgacttt ccattctaag cactttgagg atgagtttct |
| 2401 | agcttcaata gaccaaggac tctctcctag gcctctgtat tcctttcaac agctccactg |
| 2461 | tcaagagagc cagagagagc ttctgggtgg cccagctgtg aaatttctga gtcccttagg |
| 2521 | gatagcccta aacgaaccag atcatcctga ggacagccaa gaggttttgc cttctttcaa |
| 2581 | gacaagcaac agtactcaca taggctgtgg gcaatggtcc tgtctctcaa gaatcccctg |
| 2641 | ccactcctca cacccaccct gggcccatat tcatttccat ttgagttgtt cttattgagt |
| 2701 | catccttcct gtggtagcgg aactcactaa ggggcccatc tggacccgag gtattgtgat |
| 2761 | gataaattct gagcacctac cccatcccca gaagggctca gaaataaaat aagagccaag |
| 2821 | tctagtcggt gtttcctgtc ttgaaacaca atactgttgg ccctggaaga atgcacagaa |
| 2881 | tctgtttgta aggggatatg cacagaagct gcaagggaca ggaggtgcag gagctgcagg |
| 2941 | cctcccccac ccagcctgct ctgccttggg gaaaaccgtg ggtgtgtcct gcaggccatg |
| 3001 | caggcctggg acatgcaagc ccataaccgc tgtggcctct tggttttaca gatacgaacc |
| 3061 | taaactttca aaacctgtca gtgattgggt tccgaatcct cctcctgaaa gtggccgggt |
| 3121 | ttaatctgct catgacgctg cggctgtggt ccagctgagg tgaggggcct tgaagctggg |
| 3181 | agtggggttt agggacgcgg gtctctgggt gcatcctaag ctctgagagc aaacctccct |
| 3241 | gcagggtctt gcttttaagt ccaaagcctg agcccaccaa actctcctac ttcttcctgt |
| 3301 | tacaaattcc tcttgtgcaa taataatggc ctgaaacgct gtaaaatatc ctcatttcag |
| 3361 | ccgcctcagt tgcacttctc ccctatgagg taggaagaac agttgtttag aaacgaagaa |
| 3421 | actgaggccc cacagctaat gagtggagga agagagacac ttgtgtacac cacatgcctt |
| 3481 | gtgttgtact tctctcaccg tgtaacctcc tcatgtcctc tctccccagt acggctctct |
| 3541 | tagctcagta gaaagaagac attacactca tattacaccc caatcctggc tagagtctcc |
| 3601 | gcaccctcct cccccagggt ccccagtcgt cttgctgaca actgcatcct gttccatcac |
| 3661 | catcaaaaaa aaactccagg ctgggtgcgg gggctcacac ctgtaatccc agcactttgg |
| 3721 | gaggcagagg caggaggagc acaggagctg gagaccagcc tgggcaacac agggagaccc |
| 3781 | cgcctctaca aaaagtgaaa aaattaacca ggtgtggtgc tgcacacctg tagtcccagc |
| 3841 | tacttaagag gctgagatgg gaggatcgct tgagccctgg aatgttgagg ctacaatgag |
| 3901 | ctgtgattgc gtcactgcac tccagcctgg aagacaaagc aagatcctgt ctcaaataat |
| 3961 | aaaaaaaata agaactccag ggtacatttg ctcctagaac tctaccacat agccccaaac |
| 4021 | agagccatca ccatcacatc cctaacagtc ctgggtcttc ctcagtgtcc agcctgactt |
| 4081 | ctgttcttcc tcattccaga tctgcaagat tgtaagacag cctgtgctcc ctcgctcctt |
| 4141 | cctctgcatt gcccctcttc tccctctcca aacagaggga actctcctac ccccaaggag |
| 4201 | gtgaaagctg ctaccacctc tgtgcccccc cggcaatgcc accaactgga tcctacccga |
| 4261 | atttatgatt aagattgctg aagagctgcc aaacactgct gccaccccct ctgttccctt |
| 4321 | attgctgctt gtcactgcct gacattcacg gcagaggcaa ggctgctgca gcctcccctg |
| 4381 | gctgtgcaca ttccctcctg ctccccagag actgcctccg ccatcccaca gatgatggat |
| 4441 | cttcagtggg ttctcttggg ctctaggtcc tgcagaatgt tgtgaggggt ttattttttt |
| 4501 | ttaatagtgt tcataaagaa atacatagta ttcttcttct caagacgtgg ggggaaatta |
| 4561 | tctcattatc gaggccctgc tatgctgtgt atctgggcgt gttgtatgtc ctgctgccga |
| 4621 | tgccttc |

In certain embodiments, the first antigen binding chain or the second antigen binding chain comprises a constant domain comprising a native or modified TRBC polypeptide. In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO:43 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 43. SEQ ID NO:43 is provided below.

[SEQ ID NO: 43]
DLKNVFPPEVAVFEPSEAEISHTQKATLVCLATGFYPDHVELSWWVNGK
EVHSGVSTDPQPLKEQPALNDSRYCLSSRLRVSATFWQNPRNHFRCQVQ
FYGLSENDEWTQDRAKPVTQIVSAEAWGRADCGFTSESYQQGVLSATIL
YEILLGKATLYAVLVSALVLMAMVKRKDSRG

An exemplary nucleotide sequence encoding the amino acid sequence of SEQ ID NO: 43 is set forth in SEQ ID NO: 70, which is provided below.

[SEQ ID NO: 70]
GATCTGAAAAACGTGTTCCCTCCTGAAGTGGCTGTCTTTGAACCATCCG
AGGCCGAGATTTCCCATACCCAGAAAGCAACTCTGGTCTGTCTGGCCAC
TGGATTCTACCCCGATCACGTGGAACTGTCTTGGTGGGTGAACGGCAAG
GAAGTCCATTCCGGAGTCTCTACCGACCCTCAGCCCCTCAAGGAGCAGC
CTGCTCTCAACGATTCTCGGTACTGCCTGTCATCTCGACTGAGAGTGTC
TGCCACCTTCTGGCAGAACCCTAGAAACCACTTTCGGTGTCAGGTCCAG
TTTTACGGCCTGAGCGAGAACGATGAGTGGACACAGGATAGAGCCAAAC
CTGTGACACAGATTGTGAGCGCCGAGGCTTGGGGACGAGCCGATTGTGG
CTTCACATCCGAGTCTTACCAGCAGGGAGTGCTGTCTGCTACAATCCTC
TACGAAATTCTCCTGGGGAAGGCCACCCTGTACGCTGTCCTCGTGTCTG
CTCTGGTGCTCATGGCTATGGTCAAACGAAAGGACTCTAGAGGC

In certain embodiments, the TRBC polypeptide is a TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO:44 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 44. SEQ ID NO:44 is provided below.

[SEQ ID NO: 44]
LNKVFPPEV AVFEPSEAEI SHTQKATLVC LATGFFPDHV
ELSWWVNGKE VHSGVSTDPQ PLKEQPALND SRYCLSSRLR
VSATFWQNPR NHERCQVQFY GLSENDEWTQ DRAKPVTQIV
SAEAWGRADC GFTSVSYQQG VLSATILYEI LLGKATLYAV
LVSALVLMAM VKRKDF

In certain embodiments, the TRBC1 polypeptide comprises or consists of amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 81 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 81. SEQ ID NO: 81 is provided below.

[SEQ ID NO: 81]
DLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQ
FYGLSENDEWTQDRAKPVTQIVSAEAWGRADCGFTSVSYQQGVLSATIL
YEILLGKATLYAVLVSALVLMAMVKRKDF

An exemplary nucleotide sequence encoding the amino acid sequence of SEQ ID NO: 81 is set forth in SEQ ID NO: 71, which is provided below.

[SEQ ID NO: 71]
GACCTGAACAAGGTGTTCCCACCCGAGGTCGCTGTGTTTGAGCCATCAG
AAGCAGAGATCTCCCACACCCAAAAGGCCACACTGGTGTGCCTGGCCAC
AGGCTTCTTCCCCGACCACGTGGAGCTGAGCTGGTGGGTGAATGGGAAG
GAGGTGCACAGTGGGGTCAGCACAGACCCGCAGCCCCTCAAGGAGCAGC
CCGCCCTCAATGACTCCAGATACTGCCTGAGCAGCCGCCTGAGGGTCTC
GGCCACCTTCTGGCAGAACCCCCGCAACCACTTCCGCTGTCAAGTCCAG
TTCTACGGGCTCTCGGAGAATGACGAGTGGACCCAGGATAGGGCCAAAC
CCGTCACCCAGATCGTCAGCGCCGAGGCCTGGGGTAGAGCAGACTGTGG
CTTTACCTCGGTGTCCTACCAGCAAGGGGTCCTGTCTGCCACCATCCTC
TATGAGATCCTGCTAGGGAAGGCCACCCTGTATGCTGTGCTGGTCAGCG
CCCTTGTGTTGATGGCCATGGTCAAGAGAAAGGATTTC

In certain embodiments, the TRBC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by a gene of NCBI Genbank ID: 28639, NG_001333.2, range 645749 to 647196 (TRBC1, SEQ ID NO: 45), NCBI Genbank ID: 28638, NG_001333.2 range 655095 to 656583 (TRBC2, SEQ ID NO:46) or a fragment thereof, and/or may optionally comprise up to one or up to two or up to three conservative amino acid substitutions. In certain embodiments, the TRBC polypeptide comprises or consists of the amino acid sequence encoded by the nucleotide sequence of SEQ ID NO: 45. In certain embodiments, the TRBC polypeptide comprises or consists of the amino acid sequence encoded by the nucleotide sequence of SEQ ID NO: 46. SEQ ID NOS:45 and 46 are provided below.

| [SEQ ID NO: 45] | |
|---|---|
| 1 | aggacctgaa caaggtgttc ccacccgagg tcgctgtgtt tgagccatca gaagcagaga |
| 61 | tctcccacac ccaaaaggcc acactggtgt gcctggccac aggcttcttc cccgaccacg |
| 121 | tggagctgag ctggtgggtg aatgggaagg aggtgcacag tggggtcagc acagacccgc |
| 181 | agcccctcaa ggagcagccc gccctcaatg actccagata ctgcctgagc agccgcctga |
| 241 | gggtctcggc caccttctgg cagaaccccc gcaaccactt ccgctgtcaa gtccagttct |
| 301 | acgggctctc ggagaatgac gagtggaccc aggatagggc caaacccgtc acccagatcg |
| 361 | tcagcgccga ggcctggggt agagcaggtg agtggggcct ggggagatgc ctggaggaga |
| 421 | ttaggtgaga ccagctacca gggaaaatgg aaagatccag gtagcagaca agactagatc |
| 481 | caaaaagaaa ggaaccagcg cacaccatga aggagaattg ggcacctgtg gttcattctt |
| 541 | ctcccagatt ctcagcccaa cagagccaag cagctgggtc ccctttctat gtggcctgtg |
| 601 | taactctcat ctgggtggtg ccccccatcc ccctcagtgc tgccacatgc catggattgc |
| 661 | aaggacaatg tggctgacat ctgcatggca gaagaaagga ggtgctgggc tgtcagagga |
| 721 | agctggtctg ggcctgggag tctgtgccaa ctgcaaatct gactttactt ttaattgcct |
| 781 | atgaaaataa ggtctctcat ttattttcct ctccctgctt tctttcagac tgtggcttta |
| 841 | cctcgggtaa gtaagccctt ccttttcctc tccctctctc atggttcttg acctagaacc |
| 901 | aaggcatgaa gaactcacag acactggagg gtggagggtg ggagagacca gagctacctg |
| 961 | tgcacaggta cccacctgtc cttcctccgt gccaacagtg tcctaccagc aaggggtcct |
| 1021 | gtctgccacc atcctctatg agatcctgct agggaaggcc accctgtatg ctgtgctggt |
| 1081 | cagcgccctt gtgttgatgg ccatggtaag caggagggca ggatggggcc agcaggctgg |
| 1141 | aggtgacaca ctgacaccaa gcacccagaa gtatagagtc cctgccagga ttggagctgg |
| 1201 | gcagtaggga gggaagagat ttcattcagg tgcctcagaa gataacttgc acctctgtag |
| 1261 | gatcacagtg gaagggtcat gctgggaagg agaagctgga gtcaccagaa aacccaatgg |
| 1321 | atgttgtgat gagccttact atttgtgtgg tcaatgggcc ctactacttt ctctcaatcc |
| 1381 | tcacaactcc tggctcttaa taacccccaa aactttctct tctgcaggtc aagagaaagg |
| 1441 | atttctga |
| [SEQ ID NO: 46] | |
| 1 | aggacctgaa aaacgtgttc ccacccgagg tcgctgtgtt tgagccatca gaagcagaga |
| 61 | tctcccacac ccaaaaggcc acactggtat gcctggccac aggcttctac cccgaccacg |
| 121 | tggagctgag ctggtgggtg aatgggaagg aggtgcacag tggggtcagc acagacccgc |
| 181 | agcccctcaa ggagcagccc gccctcaatg actccagata ctgcctgagc agccgcctga |
| 241 | gggtctcggc caccttctgg cagaaccccc gcaaccactt ccgctgtcaa gtccagttct |
| 301 | acgggctctc ggagaatgac gagtggaccc aggatagggc caaacccgtc acccagatcg |
| 361 | tcagcgccga ggcctggggt agagcaggtg agtggggcct ggggagatgc ctggaggaga |
| 421 | ttaggtgaga ccagctacca gggaaaatgg aaagatccag gtagcggaca agactagatc |
| 481 | cagaagaaag ccagagtgga caaggtggga tgatcaaggt tcacagggtc agcaaagcac |
| 541 | ggtgtgcact tcccccacca agaagcatag aggctgaatg gagcacctca agctcattct |
| 601 | tccttcagat cctgacacct tagagctaag ctttcaagtc tccctgagga ccagccatac |
| 661 | agctcagcat ctgagtggtg tgcatcccat tctcttctgg ggtcctggtt tcctaagatc |
| 721 | atagtgacca cttcgctggc actggagcag catgagggag acagaaccag ggctatcaaa |
| 781 | ggaggctgac tttgtactat ctgatatgca tgtgtttgtg gcctgtgagt ctgtgatgta |
| 841 | aggctcaatg tccttacaaa gcagcattct ctcatccatt tttcttcccc tgttttcttt |
| 901 | cagactgtgg cttcacctcc ggtaagtgag tctctccttt ttctctctat ctttcgccgt |
| 961 | ctctgctctc gaaccagggc atggagaatc cacggacaca ggggcgtgag ggaggccaga |
| 1021 | gccacctgtg cacaggtgcc tacatgctct gttcttgtca acagagtctt accagcaagg |
| 1081 | ggtcctgtct gccaccatcc tctatgagat cttgctaggg aaggccacct tgtatgccgt |
| 1141 | gctggtcagt gccctcgtgc tgatggccat ggtaaggagg agggtgggat agggcagatg |
| 1201 | atgggggcag gggatggaac atcacacatg ggcataaagg aatctcagag ccagagcaca |
| 1261 | gcctaatata tcctatcacc tcaatgaaac cataatgaag ccagactggg gagaaaatgc |
| 1321 | agggaatatc acagaatgca tcatgggagg atggagacaa ccagcgagcc ctactcaaat |
| 1381 | taggcctcag agcccgcctc ccctgcccta ctcctgctgt gccatagccc ctgaaaccct |
| 1441 | gaaaatgttc tctcttccac aggtcaagag aaaggattcc agaggctag |

In certain embodiments, the first antigen binding chain or the second antigen binding chain comprises a constant domain comprising a native or modified TRGC polypeptide. In certain embodiments, the TRGC polypeptide is a native or modified TRGC1 polypeptide. In certain embodiments, the TRGC1 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 47, which is provided below. In certain embodiments, the TRGC1 polypeptide comprises or consists of the amino acid sequence set forth in SEQ ID NO: 47.

[SEQ ID NO: 47]
DKQLDADVSP KPTIFLPSIA ETKLQKAGTY LCLLEKFFPD
VIKIHWQEKK SNTILGSQEG NTMKINDTYM KESWLTVPEK
SLDKEHRCIV RHENNKNGVD QEIIFPPIKT DVITMDPKDN
CSKDANDTLL LQLTNTSAYY MYLLLLLKSV VYFAIITCCL
LRRTAFCCNG EKS

In certain embodiments, the TRGC polypeptide is a native or modified TRGC2 polypeptide. In certain embodiments, the TRGC2 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 48, which is provided below. In certain embodiments, the TRGC2 polypeptide comprises or consists of the amino acid sequence set forth in SEQ ID NO: 48.

[SEQ ID NO: 48]
DKQLDADVSP KPTIFLPSIA ETKLQKAGTY LCLLEKFFPD
IIKIHWQEKK SNTILGSQEG NTMKINDTYM KFSWLTVPEE
SLDKEHRCIV RHENNKNGID QEIIFPPIKT DVTTVDPKYN
YSKDANDVIT MDPKDNWSKD ANDTLLLQLT NTSAYYTYLL
LLLKSVVYFA IITCCLLRRT AFCCNGEKS

In certain embodiments, the TRGC polypeptide comprises or consists of an amino acid sequence that is at least about 85%, about 90%, about 95%, about 96%, about 97%, about 98%, about 99% or 100% homologous or identical to the amino acid sequence encoded by a transcript expressed by a gene of NCBI Genbank ID: 6966, NG_001336.2, range 108270 to 113860 (TRGC1, SEQ ID NO: 49), NCBI Genbank ID: 6967, NG_001336.2, range 124376 to 133924 (TRGC2, SEQ ID NO: 50) or a fragment thereof, and/or may optionally comprise up to one or up to two or up to three conservative amino acid substitutions. In certain embodiments, the TRGC polypeptide comprises or consists of the amino acid sequence encoded by the nucleotide sequence of SEQ ID NO: 49. In certain embodiments, the TRGC polypeptide comprises or consists of the amino acid sequence encoded by the nucleotide sequence of SEQ ID NO: 50. SEQ ID NOS: 49 and 50 are provided below.

| [SEQ ID NO: 49] | |
|---|---|
| 1 | ataaacaact tgatgcagat gtttccccca agcccactat ttttcttcct tcaattgctg |
| 61 | aaacaaagct ccagaaggct ggaacatacc tttgtcttct tgagaaattt ttccctgatg |
| 121 | ttattaagat acattggcaa gaaaagaaga gcaacacgat tctgggatcc caggagggga |
| 181 | acaccatgaa gactaacgac acatacatga aatttagctg gttaacggtg ccagaaaagt |
| 241 | cactggacaa agaacacaga tgtatcgtca gacatgagaa taataaaaac ggagttgatc |
| 301 | aagaaattat ctttcctcca ataaagacag gtatgtgttt acgcatatca tctgtcagaa |
| 361 | cacttctttg aaagtgaatg ctgcattttt tcctttcagt attaatgaaa aacaaacata |
| 421 | aatctttctt aaatattgtt acatttaatg gtagcataaa tgccctgcta cttttctata |
| 481 | gaattaaaat ggtataggtt ttggagaaaa caaaattgaa aaagttactg aaggtttgtc |
| 541 | agcctcagct ccattatcca aaataagaaa gtcacgtgct ggtttttagg gttgttagat |
| 601 | ggattaaaga aacaacatac acagaagcat ctagcaacgt gacacgtggt aaacgctcaa |
| 661 | aaagtgttct cccttctttt gatgacttta cttgatcagg aaataacata tatatgtctt |
| 721 | tcaggaatgt tctgcccaag caggagagtc actcacctca atcttgctac ccacaaagtt |
| 781 | taacctaaaa acaacgggtt cattgttgac aaaatgatgt ttatctgttg ttgacagaat |
| 841 | gatgtttatc taaaaacagt tccaattttc tatttccttt gctgagacac aaaggggagg |
| 901 | caaatgtgca aagcttgagg gtagtcttac cactgtgctt aagtgttctg atttttctag |
| 961 | tgatcagggc aaaataaaaa gtatagtaag ttccaaggca gtgaatatta tacaggagag |
| 1021 | aagttacagt tttataatgt gttttccttt acactaaatt ctaaaagtaa aaagtctttt |
| 1081 | tttttttttg acagagtttc actcttgttg cccaagcagg tgtgctatgg tatgatctca |
| 1141 | gctcactgca acctccacct cccgggttca agtgattctc ttacttcagc ctcccgacag |
| 1201 | gctgggattg caggcgcctg ccaccacacc tggctaattt ttgtgttttt agtagagatg |
| 1261 | gggtttcacc atgttggcca ggctggtctc aaattcctga cctcaagtga tccatccacc |
| 1321 | tcggcctcca agtgctggga ttatgggcgt cagccactgt gcccagccta aaagtaaaat |
| 1381 | gtctttcatg agcttcccaa ggcagctacg ttaaggagga cacttctctt aatgtcattc |
| 1441 | tacagtagat ttctaatgct ctttcttgga agtttgtttt tctgagaaaa gctaaaaata |
| 1501 | taacatggaa gtgatcatat tatataatca atgaagtgct tttcaaggag ataaaactaa |
| 1561 | tctggtccac acttgcaacc aaccttgatt gagagagaga gagaactcag gatacacttg |
| 1621 | aagattttat tatggggaac agttacttta ttctttttac ctcaatcaat gcatggaaat |
| 1681 | aagtgatagt cattttcatt tatcttttaa taaatgaagt caccatgagg aaaataaaaa |
| 1741 | gacattgaaa acccattaaa gtcagccctt aaagatattt ggacatgcag acttgataac |
| 1801 | taacgtttgc attcttgaga cttacccaaa acccatacct caagtccaag tttttagaat |
| 1861 | tcatgaaata aagatctcag tgagtgcata aaattgcgca ccagaatcat atccgtatag |
| 1921 | acaagaacac atctactaga aaaataataa accaacacac caatgcaact gtgttttctt |
| 1981 | ctgttttaaa gtatgttgtc tttgtatgca tgtttgcttc ttcctttttt tttttaacat |
| 2041 | cacagataaa ttcaactctc acctcaggtt ttattgagag aactgtcaat gtgacttggc |
| 2101 | ctctgtcttt ctagtcccag aaagaattgc actgaaatct gagctcctgt aataaaaaca |
| 2161 | accatttgct gagagtaatt aacatactga aagagatttt cttagagtac acaatggtga |
| 2221 | cattatattg cctctttata aataactttc tatctatttc tgtggattat tcctacaaag |
| 2281 | tacttttcat atgtccaatt tcttttcttc ccctacaact actgtctgaa tactggctct |
| 2341 | gctatttgct gatatgattc tcggcaagtt gcctgcactt tttaaacttt atttcctcat |
| 2401 | tcagaacatg gggccataca taatacaact cacttcagtg ttattgggga attaaacaaa |
| 2461 | aaatgcatgg gaagcattta acatagtgcc tgacacaata atgagtactc agtagatgtt |
| 2521 | agcttttatt aatattgttg ttgttatgtc cagaaacact atacctccag aaaatcatgg |
| 2581 | gtacttgctg gggacattgg ggatatgcat gatttggaaa agaatgactg ctttttttgc |
| 2641 | ttagatgaga aatttttcta agccagactc cttcaaatat gtaagattct gttgtggatt |
| 2701 | caaggactga aagaattctt ggccgagtgt ggtggcttat ccctgtaatc ccagcatttt |
| 2761 | gtgaggacaa ggcaggaaga ttgcttgagt ccaggagttt gaaaccagcc tgcgcaacat |
| 2821 | ggcgaaaccc tgtctctaca aaaaatacaa acattagctc ggagtgagtg ctgacatgtg |
| 2881 | cctgtactcc cagctactca gaaggctgag atgggaggat ctcatgagcc tggggagttt |
| 2941 | gaggcttcag tgagccgtga tgacaccgta ctatactcca ctccagcctg ggtgacagtg |
| 3001 | agaccctgcc tcaaaaaaca aacaaacaaa caaacaaaac aaaattaatc tttttgctga |
| 3061 | tgtcatgtca gcagtgtgtg ttgaaggctg taaagcagcc atttgttcag tttatttttc |
| 3121 | cattgaacaa gtatttatca aaaacatact ttgtggcagt cactatgcta ggagctatga |
| 3181 | atacagaagg aaaagtaaat gctcttggat actacactcc agttgtgata aaaaagaaaa |
| 3241 | aatgtattct tcaccaactt caacatcttg atgtgcaaaa acataataca tgaattagat |
| 3301 | ctacctaatt acacagaatt agaccaattg tttctggaat tgtgggctca tatttttaat |
| 3361 | aactgtcctc ctgcctctct gtcgacaggt tttataaata ttcatttaat tacacacaca |
| 3421 | cacacgaaca attgactagt acttgctctc attcttctag atgtcatcac aatggatccc |
| 3481 | aaagacaatt gttcaaaaga tgcaaatggt aagcttttgt gtttttccct tcctcctgat |
| 3541 | cattttgttt tgaacttctc tggcttgaaa aatcagggaa tggattttgc taggttggat |
| 3601 | gctgcagaat ggacctagtg atattttaaa ttagtccctc attttctagg agttgtatta |
| 3661 | acaaacctaa ctactgcttt ggggtatgag atgactgtaa attagagagg gtacagtggt |
| 3721 | atagtgatat gcttttaatt atttcaaaaa aaagatttta ttcattcatg tgtctttttt |
| 3781 | ctttttcttt tctttttttt ttttttttgg acagagtctt gctctgtcac ccaggctgga |
| 3841 | gtgcggtggc agtatctcag ctcaccacaa cctccgcctc ccggcttcaa gtgattctcc |
| 3901 | tgcctcagct tctcgagtag ctgggactac aggcgcgtgc caccatgccc ggctaatttt |
| 3961 | tgtattttta gtagagttgg ggtttcacca tgttggccag gatggcctcg aatttgtgac |
| 4021 | ctcgtgatct gccccctcgc cctcccgaac tgttgggatt acaggcgtga gtcactgtgc |
| 4081 | ccggcctcct gtcctgtctt ttgtttaatg actgggaaaa acatgatacc atgttgcttc |
| 4141 | tcgagttgtt ttgttttagt ctttggtctt tgctagtagc taataacacg aactagtgtt |
| 4201 | tatcaagtgc tttttacaca gaagggcttg ggctgtgttc tgcattttct tgtttaaccc |
| 4261 | tcttaaaact cctataaaat ggtacatatt tttctcccaa tttacagtcc ctttaaagca |
| 4321 | aataattata aaaatcccta tacatgtcac acagctagat ctgggatttc aaatcaggcc |
| 4381 | atcaaacaaa gagtttatgt acttagtaag ttttctgttc tttttctaca atagagtcag |
| 4441 | atagcaagaa attaccaagc caggaacctg aaacaaaacg gacatcatgt ggggctgggt |
| 4501 | gggtgcatgg gctttgcaga ctggactttc actccagctc ttttaatgat taggtgtaag |
| 4561 | tgacctacat tttgtgagca acagttttct catcagccaa caaagaataa ttacaccaga |
| 4621 | ttcacagtta ttgaagagat aaaggcatga atgtgagatg tctggcatag ggcatctcat |
| 4681 | ttagcagaca cagaatgagt acttgtttct ggctttttct ctctacatat gcacaaagaa |
| 4741 | tgcgactaga agcatgggct ctagccctgc tcaactttcc tctatttcca ataccaaggg |
| 4801 | gctctgactt aggctgccac accaggcaag gagggcagta ccacctcact tgaccaaggg |
| 4861 | cagggagtca cggacacatc acttcttgag atccttttcc acaccaagga ctgatgtttc |
| 4921 | tggaattctc actttatgaa gacaaaacat ataaatggaa attttctcag gtagagactc |
| 4981 | actcttgtag ctcattgagt aggcactagt ggtccacccc cactgtcttt acttattcct |
| 5041 | tgacatcaca tatctcttgc aaaacctcaa ataatattaa atgcaatcac ccaataatag |
| 5101 | catagccata attagaggca tttaggaaag acaggtgagt gtgccacaac tacctaacac |
| 5161 | atcagcaaat ctggattaac cactttcttt gattttccac aatgcaacct tactttttaa |
| 5221 | tagttgggaa tgttctaagt gaatttagca gaggttgtta atcaacttga aagctgaatt |
| 5281 | ctgacttgtc tgactcttgg tggtgctggt agcagtagat gtttactttt aggttttggt |
| 5341 | ggtggtggaa tatcacttca acgtaaatca tcagaaataa gtatttgtga acccctctcg |
| 5401 | cattaatgta tcttattctg taaaaagaac atgtgcaatt tctcttagat acactactgc |
| 5461 | tgcagctcac aaacacctct gcatattaca tgtacctcct cctgctcctc aagagtgtgg |
| 5521 | tctattttgc catcatcacc tgctgtctgc ttagaagaac ggctttctgc tgcaatggag |
| 5581 | agaaatcata a |
| [SEQ ID NO: 50] | |
| 1 | ataaacaact tgatgcagat gtttccccca agcccactat ttttcttcct tcgattgctg |
| 61 | aaacaaaact ccagaaggct ggaacatacc tttgtcttct tgagaaattt ttcccagata |
| 121 | ttattaagat acattggcaa gaaaagaaga gcaacacgat tctgggatcc caggagggga |
| 181 | acaccatgaa gactaacgac acatacatga aatttagctg gttaacggtg ccagaagagt |
| 241 | cactggacaa agaacacaga tgtatcgtca gacatgagaa taataaaaac ggaattgatc |
| 301 | aagaaattat ctttcctcca ataaagacag gtatgtgttt acacatatca tctgtcagaa |
| 361 | cacttctttg aaagtgaatg ctgcattttt tcctttcagt attaatgaaa aacataaatc |
| 421 | tttcttaaaa attgttacat ttaatggtag cgtaaatgcc ctgctacttt tctatagaat |
| 481 | taaaatggta taggttttgg agaaaacaaa attgaaaaag ttgctgaagg tttgtcagcc |
| 541 | tcagctccat tatccaaaat aagaaagtca cgtgctggtt tttagggttg ttagatggat |
| 601 | taaagaaaca acatacacag aagcatctag caacgtgaca cgtggtaaac gctcaaaaag |
| 661 | tgttctccct tcttttgatg actttacttg atcaggaaat aacatatata tgtctttcag |
| 721 | gaatgttctg cccaagcagg agagtcactc acctcaatct tgctacccac aaagtttaac |
| 781 | ctaaaaacaa cgggttcatt gttgacaaaa taatgtttat ctgaagataa ctgtagatca |
| 841 | tatttatctg tagataatgt ttatctgtgg agtgtggctc tacaaaacat agaatagtct |
| 901 | tggtcactgc agttttatag aggccttggg tttttcagag tttcatttta tatatcacca |
| 961 | taaagtaaca tttcataatt acaggttggt aaggcttaca tgtacaaaca ttcttccatt |
| 1021 | ttccataata aatgcatttc ctgccattgg tgaatgcagc tcaataaaca tttattgtac |
| 1081 | aattatgaca cgccaggctt agtggaaatg tggatgaaca gacaaggatg agttactgtc |
| 1141 | ctaaggatga tgcatgacag tgcagagaat atactctctt cctgatcact cagggtcact |
| 1201 | catgattcat gcgcgaggtc ccaaaacagt gcctttgatg cagattctgt acatctctag |
| 1261 | acgattggtc caagggctga atgtgctctg gcccagtggt ccagtctgtc actatatgtc |
| 1321 | aacatcctga atatgaacat aacagtccaa catctcaaga gtgggcatga aaaggactca |
| 1381 | ttttgtgctt tttcctgtgg ttaacaagtc ctttttagcc tgggggaaca agcattaaca |
| 1441 | aaatgtttga agatctttgc cacgtaccat tccaaatttc tagggtaagt ctttagcttt |
| 1501 | tcagatcctg agtttctgca atgatcaaat gtgatttgga cagttgcgtt gactttctcc |
| 1561 | tggggctata atggagtgca aaggaaacaa tggcagggaa aatgcttgct ttcaaaatgg |
| 1621 | tagcatggat gtgttcattc gtgtagttac tgtattaggt atagcctttc ctgaaactaa |
| 1681 | ctgaagtggg gttataaaaa cagtcccaat tttctatttc ctttgctgag acacaaagag |
| 1741 | gagacaaaag agcaaagctt gagggtagtt ttaccactgt gcttaagtgt tctgattttt |
| 1801 | ccagtgatca gggtgaaata aaaagcatag taagttccag ggcagtgaat accatacagg |
| 1861 | agacaagtta cagttttata atgtgtttta ctttacacta aattctaaaa gtaaaatgtc |
| 1921 | tttttttttt tccgagacag agtttcactc ttgtagccca ggcaggagtg ctatggtgtg |
| 1981 | atctcggctc acagcaacct ccacctccca gtttcaagcg attcttctgc ctcagcctcc |
| 2041 | cgagaagttg aaattacagg tgcctggcac catatctcgc taattattct atttttagta |
| 2101 | gagatcgggt tttaccatgt tggccaggct ggtctcgaac tcctgacttc aagtgatcca |
| 2161 | cccgcctcag cctcccaaag tgctgggatt acaggtgtga gtcactgtgc cggacctaac |
| 2221 | agtaaaatgt ctttcatgtg cttctcaagg caactacatt aaggaggaca catctcttaa |
| 2281 | tgtcattcta cagtagattt ctaatgctct ttcttggaag tttgtttttc tgagaagagc |
| 2341 | taaaaatata ataacatgga agtgatcata ttatataatc aatgaagtgc tttcaaagga |
| 2401 | gataaaacta acctggtctg catttgcaac cagccttgat tgagagagag agaactcagg |
| 2461 | atacacttag agattttatt atggggaata gttactttat tcattttacc tcaatcaatg |
| 2521 | catggaaata agtgacagtc attttcattt atcttttaat aaataaagtc accatgagga |
| 2581 | aaatgaaaac ccattaaagt cagtccttaa agatatttgg acatgcagac atgataacta |
| 2641 | acatttccat tcgtgagact tacccaaaac ctatacctca agtccatttc ttagaataca |
| 2701 | tgaaataaag atctcagtga gtgtataaaa ctgcacacca gaatcatatc cgtatagaca |
| 2761 | agaatacatc tactagaaaa atataaacca aaacaccaag gtgactctgt ttttttctgt |
| 2821 | tttaaaatat gttgtctttg tatgcatgtt tgcttcttcc tttttttttt taaacatcgc |
| 2881 | agataaattc aactctcacc tcagttgaga gagaactgtc aatgtgactt ggcctctctc |
| 2941 | tttctagtcc cagaaagaat tgcactgaaa tgctgagctc ctgtaataaa aatgaccatt |
| 3001 | tgctgagagt aattaacata ctgaaagaga ttttcttaga atagtgcaca atggcccaat |
| 3061 | ggtgacatta tattgtctct ttataaatta ttttctatct atttctgtgg attatttcta |
| 3121 | caaagcactt ttcatatgtc caattccttt tattccccta caagtactga ctgactactg |
| 3181 | gctctgctgt tcactgatat gactttcggc aagttgcctg cactttttaa acgttatttc |
| 3241 | ctcattcaga acatggggcc atacaaaata caactcactt cagtgttatt ggggaattaa |
| 3301 | acaaataaat gcatgggaag catttaacat agtgcctgac acaataatga gcactcagta |
| 3361 | gatgttagct tttattaata ttgttgttgc tatgtccaga aacactatac ctccagaaaa |
| 3421 | tcatgggtac ttgctgggga cgttggggat atgcatgatt ttgaaaggag tgactgctct |
| 3481 | ttactgctca gatgagaaat ttttctaagc cagactcctt caaacatgta agattctgtt |
| 3541 | gtggattcta ggactgaaag aattcttggc cgagtgtggt ggcttatcct ggtaatctca |
| 3601 | tcatttggga ggacaaggca ggaagattgc ttgagcccag gagttggaaa caagcctgga |
| 3661 | caacatggcg aaaccctgtc tctacaaaaa atacaaacat tagctggtca tgggagtgag |
| 3721 | tgcctgtact cccagctact caggaggcta agataggagg atcacctgag cctgggcagt |
| 3781 | ttgaggtttc agtgagccgt gatgacacca tactatactc cactccagcc tgggtgacag |
| 3841 | tgacatcctg cctcaaaaaa acccccaaaa ttattctttt tgctgatttc atgtcagcag |
| 3901 | tgtgtgctga aggctgtaaa gtagccactt gttctgttta tttttccatt gaacaagtat |
| 3961 | ttatcaaaaa cgtactttgt ggaaggcact gtgctaggaa ctatgcatac agaaggaaaa |
| 4021 | ccaaatgttc ttggatacta cactccagtt gtgataaaaa agaaaaaagt attcttcaca |
| 4081 | aacttcaaca ttttgatgtg caaaaacata atatatgaat tagatctacc taactacaca |
| 4141 | gaattagacc aattatttct gggattatgg gctcatattt ttaataactg tcctcctacc |
| 4201 | tctctgttga caggttttat aaatattcat ttaattacac acagtcacag acacactcag |
| 4261 | acacacacac atacacacac acacacacct tgacaaataa tgggcatgaa caattgactg |
| 4321 | gtacttgctc tcattcttct agatgtcacc acagtggatc ccaaatacaa ttattcaaag |
| 4381 | gatgcaaatg gtaagttttt gtgtttttta tttcctcctg atcattttaa gttttgaact |
| 4441 | tctctggctt gaaaaatcag ggaatggatt ttgctaggtt ggatgctgca gaatggacct |
| 4501 | aatcatattt taaattagtc cctctttttc taggagttgt attaacaaac ctaactactg |
| 4561 | cttcatgtaa gagatgactg taaattgaag ggtacagtga tatgctttca gttatttcaa |
| 4621 | aaaacagact ttactcatcc atgtgtcttt tttcttttct tttttttctt ttttgagacg |
| 4681 | gagtctcgct ctgttgaaca ggctggattg cagtgacgcg atctcacctc actacaacct |
| 4741 | ccgcctctgg agttcaagcg attctccagc ctcagcttct caagtagctg ggactacagg |
| 4801 | cacatgccac catgtccggg tcatctttgt atttttagca gagaccgggt ttcactatgt |
| 4861 | tggccaggct ggtctagaat tcctgacttc gtgatctgcc ccctcagccc tccgaagtgc |
| 4921 | tgggattaca gacgtgagtc actgtgcccg gcctaacagt aaaatgtctt tcatgcgctt |
| 4981 | ctcaaggcaa ctacgttaag gaggacactt ctcttaatgt cattctacag tagatttcta |
| 5041 | atgctctttc ttggaagttt gtttttctga gaaaagctaa aaatataaca tggaagtgat |
| 5101 | catattgtat aatcaatgaa gtgcttttca aggagataaa actaatctgg tccacgtttg |
| 5161 | caaccaacct tgattgagag agagagagaa ctcaggatac acttggagat tttattatgg |
| 5221 | ggaatagtta ctttattctt ttttcctcaa tcaattcatg gaaataagtg atagtcatat |
| 5281 | tcatttatct tttaataaat gaagtcacca tgaggaaaat aaaaagacat tgaaaaccca |
| 5341 | ttaaagttag cccttaaaga tatttggaca tgcagacttg ataactaacg tttgcattct |
| 5401 | tgagacttac ccaaaaccca tacctcaagt ccatgttttt agaattcatg aaataaagat |
| 5461 | ctcagtgagt gcataaaatt gcgcaccaga atcatatccg tatagacaag aacacatcta |
| 5521 | ctagaaaaat aataaaccaa cacaccaatg caactgtgtt ttcttctgtt ttaaaatatg |
| 5581 | ttgtctttgt atgcatgttt gcttcttcct tttttttttt taacatcaca gataaattca |
| 5641 | actctcacct caggttttat tgagagaact gtcaatgtga cttggcctct gtctttctag |
| 5701 | tcccagaaag aatcgcactg aaatgctgag ctcctgtaat aaaaatgacc atttgctgag |
| 5761 | agtaattaac atactgaaag agattttctt agagtacaca atggtgacat tatattgtct |
| 5821 | ctttataaat aactttctat ctatttctgt ggattattcc tacaaagtac ttttcatatg |
| 5881 | tccagtttct tttcttcccc tacaactacc gtctgaatac tggctctgct atttgctgat |
| 5941 | atgattctcg gcaagttgcc tgcacttttt aaactttatt tcctcattca gaacatgggg |
| 6001 | ccatgtaata ctcatgtacg tgagtattac gtaataatgc tcacttaagt gttactgggg |
| 6061 | aattaaacaa aaaaatgcat ggcaagcatt taacatagtg cctgacacaa taatgagcac |
| 6121 | tcagtagatg ttagatttta ttaatattgt tgttgttatg tccggaaaca ctatacctcc |
| 6181 | agaaaatcat gggtacttgc ttgggatgtt ggggatatgc atgatttgga aaggtatgac |
| 6241 | tgcttttttc tgcttagatg agaaattttt ctaagccaga ctccttcaaa tatgtaagat |
| 6301 | tctgttgtgg attctaggac ggaaagaatt cttggtcagg tgtggtttct tatccctgta |
| 6361 | atcccagaat tttgggagga caaggcagga agattgcttg agcccaggag tttgaaacca |
| 6421 | gcctgggcaa caagacgaaa ccctgtctct acaaaagtac ataaattagc ttggcttggt |
| 6481 | ggtgtgtgcc tgtattacca gctattcggg agactgagat gggaggatct cctgaacctg |
| 6541 | tgaagtttga ggcttcagtg agccgtgatg acaccatact atactcgact ccagcctgtg |
| 6601 | cgacagtgag actctgcgtc aaaaaaaaaa ccccaaaatt attgtttttg ctgatttcag |
| 6661 | gtcagcagtg tgtgctgaag ggtgtaaagt agccacttga tcagtttatt tttccactga |
| 6721 | acaagtattt atcaaaaaca tactttgtgg tctgtttttg ataaataaaa aggcactgtg |
| 6781 | ctaggagcca tgaatacaga aggaaaacca aatgttcttg gatactacac tccagttgtg |
| 6841 | ataaaaaaga aaaatgtatt cttcacgaac ttcaacattt tgatatgcaa aaacatagta |
| 6901 | tataaattag atctacctga ttacgtagaa tcagaccaat tatttctgga attgagggct |
| 6961 | catattttta ataactgtcc tcctgcctct ctgttgacag gttttataaa tattcattta |
| 7021 | attacacaca cacacacaca caccttgaca aataatggac atgaacaatt gactagtact |
| 7081 | tgctctcatt cttctagatg tcatcacaat ggatcccaaa gacaattggt caaaagatgc |
| 7141 | aaatggtaag cttttgtgtt tttcctttcc tcctgatcat tttaagtttt gaacttctct |
| 7201 | ggcttgaaaa atcagggaat gggccgggtg cggtggctca cgcctgtaat cccagcactt |
| 7261 | tgggaggccg aggcgggcgg atcacgaggt caggagatcg agaccatccc ggctaaaacg |
| 7321 | gtgaaacccc gtctctacta aaaatacaaa aaattagccg ggcttagtgg cgggcgcctg |
| 7381 | tagtcccagc tacttgggag gctgaggcag gagaatggcg tgaacccggg aggcggagct |
| 7441 | tgcagtgagc cgagattgcg ccactgcact ccactccagc ctgggcgaca gagcgagact |
| 7501 | ccgtctcaaa aaaaaaaaaa aaaaaaaaaa aagaaaaatc agggaatgga ttttgctagg |
| 7561 | ttggatgctg cagaatggac ctagtgatat tttaaattag tccctctttt tctaggagtt |
| 7621 | gtattaacaa acctaactac tgcttcgggt atgagatgac tgtaaattag agggtacagt |
| 7681 | gatatgcttt cagttatttc aaaaaacaga ctttattcat ccgtctgtct tttttttttt |
| 7741 | tttttttttt tttttttgag acggaggagt ctcactctat cacccaggct ggagtgcagt |
| 7801 | ggcgcgatct cggctcacca taacctccgc cttactggtt caagcgattc tccagcctca |
| 7861 | gcttctcaag tagctgggac tacaggtgca caccaccata cctggctaat ttttgtattt |
| 7921 | ttaatagaga tggggtttca ccacgctggc caggatggtc ttgaattctt gacctcgtga |
| 7981 | tctgccccct cgggctccca aacttctggg attataggcg tgagccactg tgcccggcct |
| 8041 | tctgtctttt gttataatga ctggggaaaa catgatacca tgttgcttct tgagttgttt |
| 8101 | tgttttagtc tttggtcttt gctagtagct aataacacga actagtgttt atcaagtgct |
| 8161 | ttttacacag aagggcttgt tctgcatttt ctagtttaat catcttaata ctcctataaa |
| 8221 | gtagtacaat atattttctc ccattttaca gtccctttaa agtaaataac tataaaaatc |
| 8281 | ccttatacat gtcacacagc taggtctggc atttcaaatc aggacatcaa acaaagaatt |
| 8341 | cgtgcagtta ctaagtcctc tattttttct acaatagaaa aaatagcaag aattacagat |
| 8401 | agcaagacat tacaaggcag gaatctgaaa cgaaagggac ataatgtggg gctgggtggg |
| 8461 | tgcatgagct ttgcagacta gactttcatt ccagctcttt taatgattag gtgtaagtga |
| 8521 | cctacatttt gtgagtaaca gttttctcat cagccaacta agaataatta caccagattc |
| 8581 | acagttattg aagagataag ggcatgaatg tgagatgtct ggcgtagggt atctcattta |
| 8641 | gcagacacag aatgaatact tgtttctggc tttttctctc tacatatgca caaagaatgt |
| 8701 | gactagaagc attggctcta gccctgctca actttcctct atttccaata ccaaggggct |
| 8761 | ctgacttagg ctgccacacc aggcaaggag gggcagtacc acctcacttg accaagggca |
| 8821 | gggagtcacg gacacatcac ttcctgagat ccttttccac accaaggact gatgtttctg |
| 8881 | gaattctcac tttatgaaga caaaacatat aaatggaaat ttctgcagga agagactcac |
| 8941 | tcttgtagct cattgagtag gcactagtgg tccaccccca ctgtctttac ttattccttg |
| 9001 | acatcacata tctcttgtaa aacctcaaat aatgttaaat gcaatcaccc aataatagca |
| 9061 | tagccataat tagaggcatt taggaaagac aggtgagtgt gccacaacta cctaacacat |
| 9121 | cagcaaatct ggattaacca ctttctttga ttttccacaa tgcaacctta ctttttaata |
| 9181 | gttgggaatg ttctaagtga atttagcaga ggttgttaat caacttgaaa gctgaattct |
| 9241 | gacttgtctg actcttggtg gtgctggtag cagtagatgt ttacttttag gttttggtgg |
| 9301 | tggtggaata tcacttcaac gtaaatcatc agaaataagt atttgtgaac ccctctcgca |
| 9361 | ttaatatatc ttattctgta aaaagaacat gtgcaatttc tcttagatac actactgctg |
| 9421 | cagctcacaa acacctctgc atattacacg tacctcctcc tgctcctcaa gagtgtggtc |
| 9481 | tattttgcca tcatcacctg ctgtctgctt agaagaacgg ctttctgctg caatggagag |
| 9541 | aaatcataa |

In certain embodiments, the first antigen binding chain or the second antigen binding chain comprises a constant domain comprising a native or modified TRDC polypeptide. In certain embodiments, the TRDC polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 51, which is provided below. In certain embodiments, the TRDC polypeptide comprises or consists of the amino acid sequence set forth in SEQ ID NO: 51.

[SEQ ID NO: 51]
SQPHTKPSVF VMKNGTNVAC LVKEFYPKDI RINLVSSKKI
TEFDPAIVIS PSGKYNAVKL GKYEDSNSVT CSVQHDNKTV
HSTDFEVKTD STDHVKPKET ENTKQPSKSC HKPKAIVHTE
KVNMMSLTVL GLRMLFAKTV AVNELLTAKL FFL

In certain embodiments, the TCR like fusion molecule comprises a hinge/spacer region that links the first antigen binding chain to the constant domain. In certain embodiments, the TCR like fusion molecule comprises a hinge/spacer region that links the second antigen binding chain to the constant domain. The hinge/spacer region can be flexible enough to allow the antigen binding chain to orient in different directions to facilitate antigen recognition. In certain embodiments, the hinge/spacer region can be the hinge region from IgG1, the CH₂CH₃ region of immunoglobulin and portions of CD3, a portion of a TCRα polypeptide, a portion of a TCRβ polypeptide, a portion of a CD28 polypeptide, a portion of a CD8 polypeptide, or a synthetic spacer sequence. In certain embodiments, the hinge/spacer region comprises a portion of a TCRα polypeptide. In certain embodiments, the hinge/spacer region comprises a portion of the variable region (TRAV), a portion of the diversity region (TRAD), a portion of the joining region (TRAJ), a portion of the constant region (TRAC), or a combination thereof. In certain embodiments, the hinge/spacer region comprises a portion of the TRAJ region and a portion of the TRAC region of the TCRα polypeptide. In certain embodiments, the hinge/spacer region comprises or consists of the amino acid sequence set forth in SEQ ID NO: 64. In certain embodiments, the hinge/spacer region comprises or consists of amino acids 1 to 3 of the sequence set forth in SEQ ID NO: 64. An exemplary nucleic acid sequence encoding the amino acid sequence of SEQ ID NO: 64 is set forth in SEQ ID NO: 65. SEQ ID Nos: 64 and 65 are provided below.

[SEQ ID NO: 64]
IPNIQNPDP
[SEQ ID NO: 65]
ATTCCCAATATCCAGAACCCTGACCCTGCC

In certain embodiments, the hinge/spacer region comprises a portion of a TCRβ polypeptide. In certain embodiments, the hinge/spacer region comprises a portion of the variable region (TRBV), a portion of the diversity region (TRBD), a portion of the joining region (TRBJ), a portion of the constant region (TRBC), or a combination thereof. In certain embodiments, the hinge/spacer region comprises a portion of the TRBJ region and a portion of the TRAC region (C) of the TCRβ polypeptide. In certain embodiments, the hinge/spacer region comprises or consists of the amino acid sequence set forth in SEQ ID NO: 66. In certain embodiments, the hinge/spacer region comprises or consists of amino acid 1 to 2 of the sequence set forth in SEQ ID NO: 66. An exemplary nucleic acid sequence encoding the amino acid sequence of SEQ ID NO: 66 is set forth in SEQ ID NO: 67. SEQ ID Nos: 66 and 67 are provided below.

[SEQ ID NO: 66]
LEDLKNVEPPE
[SEQ ID NO: 67]
CTGGAGGATCTGAAAAACGTGTTCCCTCCTGAA

In certain embodiments, the antigen binding chain does not comprise an intracellular domain. In certain embodiments, the antigen binding chain is capable of associating with a CD3ζ polypeptide. In certain embodiments, the antigen binding chain associating with the CD3ζ polypeptide via the constant domain. In certain embodiments, the CD3ζ polypeptide is endogenous. In certain embodiments, the CD3ζ polypeptide is exogenous. In certain embodiments, binding of the antigen binding chain to a target antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the CD3ζ polypeptide comprises or consists of an amino acid sequence that is at least about 85%, about 90%, about 95%, about 96%, about 97%, about 98%, about 99% or about 100% homologous to the amino acid sequence set forth in SEQ ID NO: 12 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 12, which is at least about 20, or at least about 30, or at least about 40, or at least about 50, and up to about 164 amino acids in length. In certain embodiments, the CD3ζ comprises or consists of the amino acid sequence of amino acids 1 to 164, 1 to 50, 50 to 100, 52 to 164, 100 to 150, or 150 to 164 of SEQ ID NO: 12. In certain embodiments, the CD3ζ polypeptide comprises or consists of amino acids 52 to 164 of SEQ ID NO: 12.

In certain embodiments, the CD3ζ polypeptide comprises or consists of an amino acid sequence that is at least about 85%, about 90%, about 95%, about 96%, about 97%, about 98%, about 99% or about 100% homologous or identical to SEQ ID NO: 13 or a fragment thereof, and/or may optionally comprise up to one or up to two or up to three conservative amino acid substitutions. In certain embodiments, the CD3ζ polypeptide comprises or consists of the amino acid sequence set forth in SEQ ID NO: 13.

In certain embodiments, the TCR like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3ζ polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR like fusion molecule is capable of associating with a CD3 complex (also known as “T-cell co-receptor”). In certain embodiments, the TCR like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR like fusion molecule replaces a native and/or an endogenous TCR in the CD3/TCR complex. In certain embodiments, the CD3 complex comprises a CD3γ chain, a CD3δ chain, and two CD3ε chains.

In certain embodiments, the CD3γ chain comprises or consists of an amino acid sequence that is at least about 85%, about 90%, about 95%, about 96%, about 97%, about 98%, about 99% or about 100% homologous or identical to the amino acid sequence having a NCBI reference number: NP_000064.1 (SEQ ID NO: 52) or a fragment thereof, and/or may optionally comprise up to one or up to two or up to three conservative amino acid substitutions. SEQ ID NO: 52 is provided below.

[SEQ ID NO: 52]
MEQGKGLAVL ILAIILLQGT LAQSIKGNHL VKVYDYQEDG
SVLLTCDAEA KNITWEKDGK MIGFLTEDKK KWNLGSNAKD
PRGMYQCKGS QNKSKPLQVY YRMCQNCIEL NAATISGELE
AEIVSIFVLA VGVYFIAGQD GVRQSRASDK QTLLPNDQLY
QPLKDREDDQ YSHLQGNQLR RN

In certain embodiments, the CD3δ chain comprises or consists of an amino acid sequence that is at least about 85%, about 90%, about 95%, about 96%, about 97%, about 98%, about 99% or about 100% homologous or identical to the amino acid sequence having a NCBI reference numbers: NP_000723.1 (SEQ ID NO: 53) or a fragment thereof, or the amino acid sequence having a NCBI reference numbers: NP_001035741.1 (SEQ ID NO: 54) or a fragment thereof, and/or may optionally comprise up to one or up to two or up to three conservative amino acid substitutions. SEQ ID NOS: 53 and 54 are provided below.

[SEQ ID NO: 53]
MEHSTFLSGL VLATLLSQVS PFKIPIEELE DRVFVNCNTS
ITWVEGTVGT LLSDITRLDL GKRILDPRGI YRCNGTDIYK
DKESTVQVHY RMCQSCVELD PATVAGIIVT DVIATLLLAL
GVFCFAGHET GRLSGAADTQ ALLRNDQVYQ PLRDRDDAQY
SHLGGNWARN K
[SEQ ID NO: 54]
MEHSTELSGL VLATLLSQVS PFKIPIEELE DRVFVNCNTS
ITWVEGTVGT LLSDITRLDL GKRILDPRGI YRCNGTDIYK
DKESTVQVHY RTADTQALLR NDQVYQPLRD RDDAQYSHLG
GNWARNK

In certain embodiments, the CD3ε chain comprises or consists of an amino acid sequence that is at least about 85%, about 90%, about 95%, about 96%, about 97%, about 98%, about 99% or about 100% homologous or identical to the amino acid sequence having a NCBI reference number: NP_000724.1 (SEQ ID NO: 55) or a fragment thereof, and/or may optionally comprise up to one or up to two or up to three conservative amino acid substitutions. SEQ ID NO: 55 is provided below.

[SEQ ID NO: 55]
mqsgthwrvl glcllsvgvw gqdgneemgg itqtpykvsi
sgttviltcp qypgseilwq hndkniggde ddknigsded
hlslkefsel eqsgyyvcyp rgskpedanf ylylrarvce
ncmemdvmsv ativivdici tggllllvyy wsknrkakak
pvtrgagagg rqrgqnkerp ppvpnpdyep irkgqrdlys
glnqrri

In certain embodiments, the TCR like fusion molecule exhibits a greater antigen sensitivity than a CAR targeting the same antigen. In certain embodiments, the TCR like fusion molecule is capable of inducing an immune response when binding to an antigen that has a low density on the surface of a tumor cell. In certain embodiments, cells comprising the TCR like fusion molecule can be used to treat a subject having tumor cells with a low expression level of a surface antigen, e.g., from a relapse of a disease, wherein the subject received treatment which leads to residual tumor cells. In certain embodiments, the tumor cells have a low density of a target molecule on the surface of the tumor cells. In certain embodiments, a target molecule having a low density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low density on the cell surface has a density of less than about 2,000 molecules per cell. In certain embodiments, a target molecule having a low density on the cell surface has a density of less than about 1,500 molecules per cell. In certain embodiments, a target molecule having a low density on the cell surface has a density of less than about 1,000 molecules per cell. In certain embodiments, a target molecule having a low density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

In certain embodiments, the first antigen-recognizing receptor is a TCR like fusion molecule that comprises a first antigen binding chain comprising a V_{H} of an antibody and a constant domain comprising a TRBC polypeptide; and a second antigen binding chain comprising a V_{L} of an antibody and a constant domain comprising a TRAC polypeptide. In certain embodiments, the first antigen binding chain is designated as “V_{H}-TRBC chain”. In certain embodiments, the second antigen binding chain is designated as “V_{L}-TRAC chain”. In certain embodiments, the first antigen binding chain comprises a hinge region between the V_{H} and the TRBC polypeptide. In certain embodiments, the hinge region comprises or consists of the amino acid sequence set forth in SEQ ID NO: 66. In certain embodiments, the second antigen binding chain comprises a hinge region between the V_{L} and the TRAC polypeptide. In certain embodiments, the hinge region comprises or consists of the amino acid sequence set forth in SEQ ID NO: 64. In certain embodiments, the first antigen binding chain and the second antigen binding chain bind to CD70 (e.g., human CD70). In certain embodiments, the V_{H}-TRBC chain comprises the amino acid sequence set forth in SEQ ID NO: 72. An exemplary nucleotide sequence encoding the amino acid sequence of SEQ ID NO: 72 is set forth in SEQ ID NO: 73. In certain embodiments, the V_{L}-TRAC chain comprises the amino acid sequence set forth in SEQ ID NO: 74. An exemplary nucleotide sequence encoding the amino acid sequence of SEQ ID NO: 74 is set forth in SEQ ID NO: 75. SEQ ID NOs: 72-75 are provided below.

[SEQ ID NO: 72]
QVQLVESGGGVVQPGRSLRLSCAASGFTFSSYIMHWVRQAPGKGLEWVA
VISYDGRNKYYADSVKGRFTISRDNSKNTLYLQMNSLRAEDTAVYYCAR
DTDGYDFDYWGQGTLVTVLEDLKNVFPPEVAVFEPSEAEISHTQKATLV
CLATGFYPDHVELSWWVNGKEVHSGVSTDPQPLKEQPALNDSRYCLSSR
LRVSATFWQNPRNHFRCQVQFYGLSENDEWTQDRAKPVTQIVSAEAWGR
ADCGFTSESYQQGVLSATILYEILLGKATLYAVLVSALVLMAMVKRKDS
RG
[SEQ ID NO: 73]
CAGGTGCAGCTGGTGGAGTCTGGGGGAGGCGTGGTCCAGCCTGGGAGGT
CCCTGAGACTCTCCTGTGCAGCCTCTGGATTTACCTTCAGTAGCTATAT
TATGCACTGGGTCCGCCAGGCTCCAGGCAAGGGGCTGGAGTGGGTGGCA
GTTATATCATATGATGGAAGAAACAAATACTACGCAGACTCCGTGAAGG
GCCGATTCACCATCTCCAGAGACAATTCCAAGAACACGCTGTATCTGCA
AATGAACAGCCTGAGAGCTGAGGACACGGCTGTGTATTACTGTGCGAGA
GATACGGATGGCTACGATTTTGACTACTGGGGCCAGGGAACCCTGGTCA
CCGTCCTGGAGGATCTGAAAAACGTGTTCCCTCCTGAAGTGGCTGTCTT
TGAACCATCCGAGGCCGAGATTTCCCATACCCAGAAAGCAACTCTGGTC
TGTCTGGCCACTGGATTCTACCCCGATCACGTGGAACTGTCTTGGTGGG
TGAACGGCAAGGAAGTCCATTCCGGAGTCTCTACCGACCCTCAGCCCCT
CAAGGAGCAGCCTGCTCTCAACGATTCTCGGTACTGCCTGTCATCTCGA
CTGAGAGTGTCTGCCACCTTCTGGCAGAACCCTAGAAACCACTTTCGGT
GTCAGGTCCAGTTTTACGGCCTGAGCGAGAACGATGAGTGGACACAGGA
TAGAGCCAAACCTGTGACACAGATTGTGAGCGCCGAGGCTTGGGGACGA
GCCGATTGTGGCTTCACATCCGAGTCTTACCAGCAGGGAGTGCTGTCTG
CTACAATCCTCTACGAAATTCTCCTGGGGAAGGCCACCCTGTACGCTGT
CCTCGTGTCTGCTCTGGTGCTCATGGCTATGGTCAAACGAAAGGACTCT
AGAGGC
[SEQ ID NO: 74]
EIVLTQSPATLSLSPGERATLSCRASQSVSSYLAWYQQKPGQAPRLLIY
DASNRATGIPARFSGSGSGTDFTLTISSLEPEDFAVYYCQQRTNWPLTF
GGGTKVEIIPNIQNPDPAVYQLRDSKSSDKSVCLFTDFDSQTNVSQSKD
SDVYITDKTVLDMRSMDFKSNSAVAWSNKSDFACANAFNNSIIPEDTFF
PSPESSCDVKLVEKSFETDTNLNFQNLSVIGFRILLLKVAGENLLMTLR
LWSS
[SEQ ID NO: 75]
GAAATTGTGTTGACACAGTCTCCAGCCACCCTGTCTTTGTCTCCAGGGG
AAAGAGCCACCCTCTCCTGCAGGGCCAGTCAGAGTGTTAGCAGCTACTT
AGCCTGGTACCAACAGAAACCTGGCCAGGCTCCCAGGCTCCTCATCTAT
GATGCATCCAACAGGGCCACTGGCATCCCAGCCAGGTTCAGTGGCAGTG
GGTCTGGGACAGACTTCACTCTCACCATCAGCAGCCTAGAGCCTGAAGA
TTTTGCAGTTTATTACTGTCAGCAGCGTACCAACTGGCCGCTCACTTTC
GGCGGAGGGACCAAGGTGGAGATCATTCCCAATATCCAGAACCCTGACC
CTGCCGTGTACCAGCTGAGAGACTCTAAATCCAGTGACAAGTCTGTCTG
CCTATTCACCGATTTTGATTCTCAAACAAATGTGTCACAAAGTAAGGAT
TCTGATGTGTATATCACAGACAAAACTGTGCTAGACATGAGGTCTATGG
ACTTCAAGAGCAACAGTGCTGTGGCCTGGAGCAACAAATCTGACTTTGC
ATGTGCAAACGCCTTCAACAACAGCATTATTCCAGAAGACACCTTCTTC
CCCAGCCCAGAAAGTTCCTGTGATGTCAAGCTGGTCGAGAAAAGCTTTG
AAACAGATACGAACCTAAACTTTCAAAACCTGTCAGTGATTGGGTTCCG
AATCCTCCTCCTGAAAGTGGCCGGGTTTAATCTGCTCATGACGCTGCGG
CTGTGGTCCAGC

In certain embodiments, the first antigen-recognizing receptor is a TCR like fusion molecule that comprises a first antigen binding chain comprising a V_{H} of an antibody and a constant domain comprising a TRAC polypeptide; and a second antigen binding chain comprising a V_{L} of an antibody and a constant domain comprising a TRBC polypeptide. In certain embodiments, the first antigen binding chain is designated as “V_{H}-TRAC chain”. In certain embodiments, the second antigen binding chain is designated as “V_{L}-TRAC chain”. In certain embodiments, the first antigen binding chain comprises a hinge region between the V_{H} and the TRBC polypeptide. In certain embodiments, the second antigen binding chain comprises a hinge region between the V_{L} and the TRAC polypeptide. In certain embodiments, the first antigen binding chain and the second antigen binding chain bind to CD70 (e.g., human CD70).

In certain embodiments, the first antigen-recognizing receptor is a TCR like fusion molecule that binds to CD70 (e.g., human CD70) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD70. In certain embodiments, the V_{H} comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3. In certain embodiments, the V_{H} comprises the amino acid sequence set forth in SEQ ID NO: 79. In certain embodiments, the V_{L} comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6. In certain embodiments, the V_{L} comprises the amino acid sequence set forth in SEQ ID NO: 80. In certain embodiments, the TRAC polypeptide comprises the amino acid sequence set forth in SEQ ID NO: 41. In certain embodiments, the TRBC polypeptide is a TRBC2 polypeptide. In certain embodiments, the TRBC2 polypeptide comprises the amino acid sequence set forth in SEQ ID NO: 43. In certain embodiments, the V_{H}-TRBC chain comprises the amino acid sequence set forth in SEQ ID NO: 72. In certain embodiments, the V_{L}-TRAC chain comprises the amino acid sequence set forth in SEQ ID NO: 74. In certain embodiments, the TCR like fusion molecule is designated as “CD70-HIT”.

Various TCR like fusion molecules are disclosed in International Patent Application Publication No. WO2019/133969, which is incorporated by reference hereby in its entirety.

5.3.1.5. Delivery of the First Antigen-Recognizing Receptor

In certain embodiments, the first antigen-recognizing receptor is delivered to the cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the first antigen-recognizing receptor is delivered to the cell by a non-viral method. Any targeted genome editing methods can also be used to deliver the first antigen-recognizing receptor to the cell. In certain embodiments, the first antigen-recognizing receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the first antigen-recognizing receptor to the cell.

In certain embodiments, the cell is a T cell, and the first antigen-recognizing receptor is integrated at a locus within the genome of the T cell. Non-limiting examples of loci include a TRAC locus, a TRBC locus, a TRDC locus, and a TRGC locus. In certain embodiments, the locus is a TRAC locus or a TRBC locus. In certain embodiments, the cell is a T cell, and the first antigen-recognizing receptor is integrated at a TRAC locus. Methods of targeting a CAR to a site within the genome of T cell are disclosed in WO2017180989 and Eyquem et al., Nature. (2017 Mar. 2); 543(7643): 113-117, both of which are incorporated by reference in their entireties. In certain embodiments, the cell is a T Cell, the first antigen-recognizing receptor is a TCR, and the first antigen-recognizing receptor is integrated at a TRAC locus. In certain embodiments, the cell further comprises a gene disruption of a TRBC locus. In certain embodiments, the gene disruption of a TRBC locus results in knockout of TRBC locus.

In certain embodiments, the cell is a T cell, the first antigen-recognizing receptor is a recombinant TCR, and the first antigen-recognizing receptor is integrated at a TRAC locus, wherein the recombinant TCR comprises a first antigen binding chain that comprises a V_{H} of an antibody and a constant domain comprising a TRAC or TRBC polypeptide and a second antigen binding chain that comprises a V_{L} of an antibody and a constant domain comprising a TRAC or TRBC polypeptide, wherein the recombinant TCR binds to the first antigen in an HLA-independent manner. In certain embodiments, the first antigen is CD70. In certain embodiments, the cell further comprises a gene disruption of a TRBC locus. In certain embodiments, the gene disruption of a TRBC locus results in knockout of TRBC locus. In certain embodiments, the cell further comprises a gene disruption of a TRAC locus. In certain embodiments, the gene disruption of a TRAC locus results in knockout of TRAC locus.

In certain embodiments, the first antigen-recognizing receptor is integrated into a CD70 locus of the cell, e.g., in order to generate a gene disruption of the CD70 locus (e.g., a knockout of the CD70 locus). The first antigen-recognizing receptor can be integrated to the coding region of the CD70 locus and/or the non-coding region of the CD70 locus. In certain embodiments, the first antigen-recognizing receptor is integrated to the coding region of the CD70 locus. In certain embodiments, the first antigen-recognizing receptor is integrated to exon 1, exon 2, or exon 3 of the CD70 locus. In certain embodiments, the first antigen-recognizing receptor is integrated to exon 1 of the CD70 locus.

5.3.2. Co-Stimulatory Ligands

In certain embodiments, a presently disclosed cell comprising a presently disclosed gene disruption of a CD70 locus and a presently disclosed first antigen-recognizing receptor further comprises at least one recombinant or exogenous co-stimulatory ligand. For example, a presently disclosed cell can be further transduced with at least one co-stimulatory ligand, such that the cell expresses or is induced to express the first antigen-recognizing receptor and the at least one co-stimulatory ligand. The at least one co-stimulatory ligand provides a co-stimulation signal to the cell.

Non-limiting examples of co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Non-limiting examples of TNF superfamily members include nerve growth factor (NGF), CD40L (also known as “CD154”), 4-1BBL, TNF-α, OX40L, CD70, Fas ligand (FasL), CD30L, tumor necrosis factor beta (TNFβ)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Non-limiting examples of immunoglobulin superfamily ligands include CD80, CD86, and ICOSLG. In certain embodiments, the at least one co-stimulatory ligand is selected from the group consisting of 4-1BBL, CD80, CD86, CD70, GITRL, CD40L, OX40L, CD30L, TNFRSF14, ICOSLG, TRAIL, and combinations thereof.

In certain embodiments, the cell further comprises one exogenous co-stimulatory ligand that is 4-1BBL. In certain embodiments, the co-stimulatory ligand is human 4-1BBL. In certain embodiments, the 4-1BBL comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a Uniprot Reference No: P41273-1 (SEQ ID NO: 76) or a fragment thereof, and/or may optionally comprise up to one or up to two or up to three conservative amino acid substitutions. In certain embodiments, the 4-1BBL comprises or consists of an amino acid sequence that is a consecutive portion of the amino acid sequence of SEQ ID NO: 76. SEQ ID NO: 76 is provided below.

[SEQ ID NO: 76]
MEYASDASLDPEAPWPPAPRARACRVLPWALVAGLLLLLLLAAACAVFL
ACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSA
FGFQGRLLHLSAGQRLGVHLHTEARARHAWQLTQGATVLGLFRVTPEIP
AGLPSPRSE

An exemplary nucleotide sequence encoding the amino acid sequence of SEQ ID NO: 76 is set forth in SEQ ID NO: 77.

[SEQ ID NO: 77]
ATGGAATACGCCTCTGACGCTTCACTGGACCCCGAAGCCCCGTGGCCTC
CCGCGCCCCGCGCTCGCGCCTGCCGCGTACTGCCTTGGGCCCTGGTCGC
GGGGCTGCTGCTGCTGCTGCTGCTCGCTGCCGCCTGCGCCGTCTTCCTC
GCCTGCCCCTGGGCCGTGTCCGGGGCTCGCGCCTCGCCCGGCTCCGCGG
CCAGCCCGAGACTCCGCGAGGGTCCCGAGCTTTCGCCCGACGATCCCGC
CGGCCTCTTGGACCTGCGGCAGGGCATGTTTGCGCAGCTGGTGGCCCAA
AATGTTCTGCTGATCGATGGGCCCCTGAGCTGGTACAGTGACCCAGGCC
TGGCAGGCGTGTCCCTGACGGGGGGCCTGAGCTACAAAGAGGACACGAA
GGAGCTGGTGGTGGCCAAGGCTGGAGTCTACTATGTCTTCTTTCAACTA
GAGCTGCGGCGCGTGGTGGCCGGCGAGGGCTCAGGCTCCGTTTCACTTG
CGCTGCACCTGCAGCCACTGCGCTCTGCTGCTGGGGCCGCCGCCCTGGC
TTTGACCGTGGACCTGCCACCCGCCTCCTCCGAGGCTCGGAACTCGGCC
TTCGGTTTCCAGGGCCGCTTGCTGCACCTGAGTGCCGGCCAGCGCCTGG
GCGTCCATCTTCACACTGAGGCCAGGGCACGCCATGCCTGGCAGCTTAC
CCAGGGCGCCACAGTCTTGGGACTCTTCCGGGTGACCCCCGAAATCCCA
GCCGGACTCCCTTCACCGAGGTCGGAA

In certain embodiments, the cell further comprises one exogenous co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_005182 (SEQ ID NO: 56) or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 56. SEQ ID NO: 56 is provided below.

| [SEQ ID NO: 56] | |
|---|---|
| 1 | MGHTRRQGTS PSKCPYLNFF QLLVLAGLSH FCSGVIHVTK EVKEVATLSC GHNVSVEELA |
| 61 | QTRIYWQKEK KMVLTMMSGD MNIWPEYKNR TIFDITNNLS IVILALRPSD EGTYECVVLK |
| 121 | YEKDAFKREH LAEVTLSVKA DEPTPSISDF EIPTSNIRRI ICSTSGGFPE PHLSWLENGE |
| 181 | ELNAINTTVS QDPETELYAV SSKLDENMTT NHSFMCLIKY GHLRVNOTEN WNTTKQEHFP |
| 241 | DNLLPSWAIT LISVNGIFVI CCLTYCFAPR CRERRRNERL RRESVRPV |

An exemplary nucleotide sequence encoding the amino acid sequence of SEQ ID NO: 56 is set forth in SEQ ID NO: 78. SEQ ID NO: 78 is provided below.

[SEQ ID NO: 78]
ATGGGCCACACACGGAGGCAGGGAACATCACCATCCAAGTGTCCATACC
TCAATTTCTTTCAGCTCTTGGTGCTGGCTGGTCTTTCTCACTTCTGTTC
AGGTGTTATCCACGTGACCAAGGAAGTGAAAGAAGTGGCAACGCTGTCC
TGTGGTCACAATGTTTCTGTTGAAGAGCTGGCACAAACTCGCATCTACT
GGCAAAAGGAGAAGAAAATGGTGCTGACTATGATGTCTGGGGACATGAA
TATATGGCCCGAGTACAAGAACCGGACCATCTTTGATATCACTAATAAC
CTCTCCATTGTGATCCTGGCTCTGCGCCCATCTGACGAGGGCACATACG
AGTGTGTTGTTCTGAAGTATGAAAAAGACGCTTTCAAGCGGGAACACCT
GGCTGAAGTGACGTTATCAGTCAAAGCTGACTTCCCTACACCTAGTATA
TCTGACTTTGAAATTCCAACTTCTAATATTAGAAGGATAATTTGCTCAA
CCTCTGGAGGTTTTCCAGAGCCTCACCTCTCCTGGTTGGAAAATGGAGA
AGAATTAAATGCCATCAACACAACAGTTTCCCAAGATCCTGAAACTGAG
CTCTATGCTGTTAGCAGCAAACTGGATTTCAATATGACAACCAACCACA
GCTTCATGTGTCTCATCAAGTATGGACATTTAAGAGTGAATCAGACCTT
CAACTGGAATACAACCAAGCAAGAGCATTTTCCTGATAACCTGCTCCCA
TCCTGGGCCATTACCTTAATCTCAGTAAATGGAATTTTTGTGATATGCT
GCCTGACCTACTGCTTTGCCCCAAGATGCAGAGAGAGAAGGAGGAATGA
GAGATTGAGAAGGGAAAGTGTACGCCCTGTA

In certain embodiments, the cell further comprises two exogenous co-stimulatory ligands that are 4-1BBL and CD80. In certain embodiments, the cell further comprises two exogenous co-stimulatory ligands that are 4-1BBL and CD80, wherein the 4-1BBL comprises or consists of the amino acid sequence set forth in SEQ ID NO: 76, and the CD80 comprises or consists of the amino acid sequence set forth in SEQ ID NO: 56.

Receptor-comprising cells comprising at least one exogenous co-stimulatory ligand are described in U.S. Pat. No. 8,389,282, which is incorporated by reference in its entirety.

5.3.3. Fusion Polypeptides

In certain embodiments, a presently disclosed cell comprising a presently disclosed gene disruption of a CD70 locus and a presently disclosed first antigen-recognizing receptor further comprises a fusion polypeptide. For example, a presently disclosed cell can be further transduced with the fusion polypeptide, such that the cell expresses or is induced to express the first antigen-recognizing receptor and the fusion polypeptide. The fusion polypeptide provides a co-stimulation signal to the cell. The fusion polypeptides are capable of enhancing the activity and/or efficacy of a cell comprising the first antigen-recognizing receptor (e.g., a CAR or a TCR like fusion molecule). In certain embodiments, the fusion polypeptide comprises a) an extracellular domain and a transmembrane domain of a co-stimulatory ligand, and b) an intracellular domain of a first co-stimulatory molecule.

Non-limiting examples of the co-stimulatory ligand include tumor necrosis factor (TNF) family members, immunoglobulin (Ig) superfamily members, and combinations thereof. The TNF family member can be selected from the group consisting of 4-1BBL, OX40L, CD70, GITRL, CD40L, and combinations thereof. The Ig superfamily member can be selected from the group consisting of CD80, CD86, ICOS ligand (ICOSLG (also known as “CD275”), and combinations thereof. In certain embodiments, the co-stimulatory ligand is selected from the group consisting of 4-1BBL, OX40L, CD70, GITRL, CD40L, CD80, CD86, ICOSLG, and combinations thereof.

In certain embodiments, the fusion polypeptide comprises an extracellular domain and a transmembrane domain of a co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 56 or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 56.

In certain embodiments, the extracellular domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 1-242 of SEQ ID NO: 56. In certain embodiments, the extracellular domain of CD80 comprises or consists of amino acids 1-242 of SEQ ID NO: 56 or a functional fragment thereof. A functional fragment can be a consecutive portion of amino acids 1-242 of SEQ ID NO: 56, which is at least about 50, at least about 75, at least about 100, at least about 125, at least about 150, at least about 175, or at least about 200, or at least about 220 amino acids in length. In certain embodiments, the functional fragment retains at least about 80%, at least about 85%, at least about 90%, at least about 95%, or at least about 100% of the primary function of the extracellular domain of CD80. Non-limiting examples of the primary functions of the extracellular domain of CD80 include binding to/interacting with CD28, binding to/interacting with CTLA-4, binding to/interacting with PD-L1, and contributing to CD80 homodimerization. In certain embodiments, an extracellular domain of CD80 comprises or consists of amino acids 1-242 of SEQ ID NO: 56).

In certain embodiments, the transmembrane domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 243-263 of SEQ ID NO: 56. In certain embodiments, the transmembrane domain of CD80 comprises or consists of amino acids 243-263 of SEQ ID NO: 56 or a fragment thereof. Such fragment can be at least about 5, at least about 10, at least about 15, or at least about 20 amino acids in length. In certain embodiments, the transmembrane domain of CD80 comprises or consists of amino acids 243-263 of SEQ ID NO: 56.

Non-limiting examples of co-stimulatory molecules include CD28, 4-1BB, OX40, ICOS, DAP-10, CD27, CD40, NKG2D, CD2, and combinations thereof.

In certain embodiments, the fusion polypeptide comprises an extracellular domain and a transmembrane domain of a co-stimulatory molecule that is 4-1BB. In certain embodiments, the co-stimulatory molecule is human 4-1BB. In certain embodiments, the 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 30 or a fragment thereof, and/or may optionally comprise up to one or up to two or up to three conservative amino acid substitutions. In certain embodiments, the 4-1BB comprises or consists of an amino acid sequence that is a consecutive portion of the amino acid sequence of SEQ ID NO: 30. In certain embodiments, the intracellular domain of 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 214-255 of SEQ ID NO: 30 or a fragment thereof, and/or may optionally comprise up to one or up to two or up to three conservative amino acid substitutions. In certain embodiments, the intracellular domain of 4-1BB comprises or consists of amino acids 214-255 of SEQ ID NO: 30 or a functional fragment thereof. Such functional fragment can be a consecutive portion of amino acids 214-255 of SEQ ID NO: 30, which is at least about 20, at least about 25, at least about 30, at least about 35, or at least about 40 amino acids in length. In certain embodiments, the functional fragment of amino acids 214-255 of SEQ ID NO: 30 retains at least about 80%, at least about 85%, at least about 90%, at least about 95%, or at least about 100% of the primary functions of the intracellular domain of 4-1BB. Non-limiting examples of the primary functions of the intracellular domain of 4-1BB include providing co-stimulatory signaling for the activation and proliferation of an immunoresponsive cell (e.g., a T cell), and interacting and activating downstream adaptors (e.g., TRAFs). In certain embodiments, the intracellular domain of 4-1BB comprises or consists of amino acids 214-255 of SEQ ID NO: 30.

In certain embodiments, the co-stimulatory molecule is CD28. In certain embodiments, the co-stimulatory molecule is human CD28. In certain embodiments, the CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 11 or a fragment thereof, and/or may optionally comprise up to one or up to two or up to three conservative amino acid substitutions. In certain embodiments, the CD28 comprises or consists of an amino acid sequence that is a consecutive portion of the amino acid sequence of SEQ ID NO: 11. In certain embodiments, the intracellular domain of CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 180 to 219 of SEQ ID NO: 11 or a fragment thereof, and/or may optionally comprise up to one or up to two or up to three conservative amino acid substitutions. In certain embodiments, the intracellular domain of CD28 comprises or consists of amino acids 180 to 219 of SEQ ID NO: 11 or a functional fragment thereof. A functional fragment of amino acids 180 to 219 of SEQ ID NO: 11 can be a consecutive portion of amino acids 180 to 219 of SEQ ID NO: 11, which is at least about 20, at least about 25, at least about 30, or at least about 35 amino acids in length. In certain embodiments, such functional fragment retains at least about 80%, at least about 85%, at least about 90%, at least about 95%, or at least about 100% of the primary function of the intracellular domain of CD28. Non-limiting examples of the primary functions of the intracellular domain of CD28 include providing co-stimulatory signaling for the activation and proliferation of an immunoresponsive cell (e.g., a T cell), and interacting with protein adaptors (e.g., PI3K, GRB2, and LCK). In certain embodiments, the intracellular domain of CD28 comprises or consists of amino acids 180 to 219 of SEQ ID NO: 11.

In certain embodiments, the fusion polypeptide comprises an intracellular domain of a second co-stimulatory molecule. In certain embodiments, the fusion polypeptide comprises an intracellular domain of a third co-stimulatory molecule. In certain embodiments, the fusion polypeptide comprises an intracellular domain of a fourth co-stimulatory molecule. In certain embodiments, the fusion polypeptide comprises an intracellular domain of a fifth co-stimulatory molecule. In certain embodiments, the first, second, third, fourth, and fifth co-stimulatory molecule can be the same or different among each other.

In certain embodiments, the fusion polypeptide comprises an extracellular domain and a transmembrane domain of a co-stimulatory ligand that is CD80, and an intracellular domain of a co-stimulatory molecule that is 4-1BB. In certain embodiments, the fusion polypeptide comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 57. In certain embodiments, the fusion polypeptide comprises or consists of the amino acid sequence set forth in SEQ ID NO: 57. SEQ ID NO: 57 is provided below.

[SEQ ID NO: 57]
MGHTRRQGTSPSKCPYLNFFQLLVLAGLSHFCSGVIHVTKEVKEVATLS
CGHNVSVEELAQTRIYWQKEKKMVLTMMSGDMNIWPEYKNRTIFDITNN
LSIVILALRPSDEGTYECVVLKYEKDAFKREHLAEVTLSVKADFPTPSI
SDFEIPTSNIRRIICSTSGGFPEPHLSWLENGEELNAINTTVSQDPETE
LYAVSSKLDFNMTTNHSFMCLIKYGHLRVNQTFNWNTTKQEHFPDNLLP
SWAITLISVNGIFVICCLTYCFKRGRKKLLYIFKQPFMRPVQTTQEEDG
CSCRFPEEEEGGCEL

In certain embodiments, the fusion polypeptide comprises an extracellular domain and a transmembrane domain of a co-stimulatory ligand that is CD80, an intracellular domain of a first co-stimulatory molecule that is 4-1BB, and an intracellular domain of a second co-stimulatory molecule that is CD28.

In certain embodiments, the fusion polypeptide comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 58. In certain embodiments, the fusion polypeptide comprises or consists of the amino acid sequence set forth in SEQ ID NO: 58. SEQ ID NO: 58 is provided below.

[SEQ ID NO: 58]
MGHTRRQGTSPSKCPYLNFFQLLVLAGLSHFCSGVIHVTKEVKEVATLS
CGHNVSVEELAQTRIYWQKEKKMVLTMMSGDMNIWPEYKNRTIFDITNN
LSIVILALRPSDEGTYECVVLKYEKDAFKREHLAEVTLSVKADFPTPSI
SDFEIPTSNIRRIICSTSGGFPEPHLSWLENGEELNAINTTVSQDPETE
LYAVSSKLDFNMTTNHSFMCLIKYGHLRVNQTFNWNTTKQEHFPDNLLP
SWAITLISVNGIFVICCLTYCFRSKRSRLLHSDYMNMTPRRPGPTRKHY
QPYAPPRDFAAYRKRGRKKLLYIFKQPFMRPVQTTQEEDGCSCRFPEEE
EGGCEL

Various modified fusion polypeptides are disclosed in International Patent Application No. PCT/US20/42753, which is incorporated by reference hereby in its entirety.

5.3.4. Second Antigen-Recognizing Receptors

In certain embodiments, a presently disclosed cell comprising a presently disclosed gene disruption of a CD70 locus and a presently disclosed first antigen-recognizing receptor further comprises a second antigen-recognizing receptor that binds to a second antigen. In certain embodiments, the second antigen-recognizing receptor is a chimeric antigen receptor (CAR) or a chimeric co-stimulating receptor (CCR).

5.3.4.1. CARs

In certain embodiments, the second antigen-recognizing receptor is a CAR, e.g., one described in Section 5.3.1.2, provided that the extracellular antigen-binding domain of the CAR binds to the second antigen. In certain embodiments, second antigen-recognizing receptor is a CAR that comprises an extracellular antigen-binding domain that binds to a second antigen, and an intracellular signaling domain. In certain embodiments, the CAR further comprises a transmembrane domain.

5.3.4.2. Second Antigens

In certain embodiments, the second antigen is a tumor antigen, e.g., one disclosed in Section 5.31.1. In certain embodiments, the second antigen is expressed on an acute myeloid leukemia (AML) tissue. In certain embodiments, the second antigen is expressed in an acute myeloid leukemia (AML) hematopoietic stem/progenitor cell (HSPC) and/or a leukemia stem cell (LSC). In certain embodiments, the AML HSPC expresses CD34. In certain embodiments, the AML LSC expresses CD34. In certain embodiments, the second antigen is not expressed or expressed at a non-detectable level in a non-malignant hematopoietic stem cell and/or a non-malignant hematopoietic progenitor cell. In certain embodiments, the second antigen is expressed in a malignant hematopoietic stem cell and/or a malignant hematopoietic progenitor cell. In certain embodiments, the second antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHCC11. In certain embodiments, the second antigen is selected from the group consisting of CD70, IL1RAP, CD33, CLEC12A, ADGRE2, CD123, and combinations thereof. In certain embodiments, the first antigen and the second antigen are different. In certain embodiments, the second antigen is IL1RAP. In certain embodiments, the first antigen is CD70 and the second antigen is IL1RAP.

In certain embodiments, the second antigen is a pathogen antigen, e.g., one disclosed in Section 5.3.1.1.

5.3.4.3. CCRs

In certain embodiments, the second antigen-recognizing receptor is a CCR. The term “chimeric co-stimulating receptor” or “CCR” refers to a chimeric receptor that binds to an antigen and provides a co-stimulatory signal, but does not provide a T-cell activation signal to a cell comprising the CCR. Various CCRs are described in US20020018783 the contents of which are incorporated by reference in their entireties. CCRs mimic co-stimulatory signals, but unlike, CARs, do not provide a T-cell activation signal. In certain embodiments, the CCR lacks a CD3ζ polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013); 31(1):71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to a second antigen and an intracellular domain that is capable of delivering a costimulatory signal to the cell but does not alone deliver an activation signal to the cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the CCR comprises an intracellular domain of CD28 or a portion thereof. In certain embodiments, the CCR comprises an intracellular domain of 4-1BB or a portion thereof. In certain embodiments, the CCR comprises an intracellular domain of CD28 or a portion thereof, and an intracellular domain of 4-1BB or a portion thereof.

In certain embodiments, the second antigen is selected so that expression of both the first antigen and the second antigen is restricted to the targeted cells (e.g., cancerous tissue or cancerous cells, or LSCs, or AML HSPCs). Similar to a CAR, the extracellular antigen-binding domain can be an scFv, a Fab, a F(ab)₂, or a fusion protein with a heterologous sequence to form the extracellular antigen-binding domain.

In certain embodiments, the cell comprising the first antigen-recognizing receptor and the second antigen-recognizing receptor (e.g., a CCR) exhibits a greater degree of cytolytic activity against cells that are positive for both the first antigen and the second antigen as compared to against cells that are singly positive for the first antigen. In certain embodiments, the cell comprising the first antigen-recognizing receptor and the second antigen-recognizing receptor (e.g., a CCR) exhibits substantially no or negligible cytolytic activity against cells that are singly positive for the first antigen.

In certain embodiments, the first antigen recognizing receptor (e.g., a CAR, a TCR, or a TCR like fusion molecule) binds to the first antigen with a low binding affinity, e.g., a dissociation constant (K_{D}) of about 1×10⁻⁸ M or more, about 5×10⁻⁸ M or more, about 1×10⁻⁷ M or more, about 5×10⁻⁷ M or more, or about 1×10⁻⁶ M or more, or from about 1×10⁻⁸ M to about 1×10⁻⁶ M. In certain embodiments, the first antigen recognizing receptor (e.g., a CAR, a TCR, or a TCR like fusion molecule) binds to the first antigen with a low binding avidity. In certain embodiments, the first antigen recognizing receptor (e.g., a CAR, a TCR, or a TCR like fusion molecule) binds to the first antigen at an epitope of low accessibility. In certain embodiments, the first antigen recognizing receptor (e.g., a CAR, a TCR, or a TCR like fusion molecule) binds to the first antigen with a binding affinity that is lower compared to the binding affinity with which the second antigen-recognizing receptor (e.g., a CCR) binds to the second antigen. In certain embodiments, the CCR binds to the second antigen with a binding affinity K_{D} of from about 1×10⁻⁹ M to about 1×10⁻⁷ M, e.g., about 1×10⁻⁷ M or less, about 1×10⁻⁸ M or less, or about 1×10⁻⁹ M or less.

5.3.4.4. Delivery of the Second Antigen-Recognizing Receptor

In certain embodiments, the second antigen-recognizing receptor is delivered to the cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the first antigen-recognizing receptor is delivered to the cell by a non-viral method. Any targeted genome editing methods can also be used to deliver the first antigen-recognizing receptor to the cell. In certain embodiments, the first antigen-recognizing receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the second antigen-recognizing receptor to the cell.

In certain embodiments, the cell is a T cell, and the second antigen-recognizing receptor is integrated at a locus within the genome of the T cell. Non-limiting examples of loci include a TRAC locus, a TRBC locus, a TRDC locus, and a TRGC locus. In certain embodiments, the locus is a TRAC locus or a TRBC locus. In certain embodiments, the cell is a T cell, and the second antigen-recognizing receptor is integrated at a TRAC locus.

In certain embodiments, the second antigen-recognizing receptor is integrated into a CD70 locus of the cell, e.g., in order to generate a gene disruption of the CD70 locus (e.g., a knockout of the CD70 locus). The second antigen-recognizing receptor can be integrated to the coding region of the CD70 locus and/or the non-coding region of the CD70 locus. In certain embodiments, the second antigen-recognizing receptor is integrated to the coding region of the CD70 locus. In certain embodiments, the second antigen-recognizing receptor is integrated to exon 1, exon 2, or exon 3 of the CD70 locus. In certain embodiments, the second antigen-recognizing receptor is integrated to exon 1 of the CD70 locus.

In certain embodiments, the cell is a T cell, the first antigen-recognizing receptor is integrated at a TRAC locus, and the second antigen-recognizing receptor is integrated at a CD70 locus. In certain embodiments, the gene disruption of the CD70 locus is produced by the integration of the second antigen-recognizing receptor at the CD70 locus. In certain embodiments, a CRISPR system is used to integrate the first antigen-recognizing receptor to the TRAC locus. In certain embodiments, a CRISPR system is used to integrate the second antigen-recognizing receptor to the CD70 locus.

In certain embodiments, the cell is a T cell, the first antigen-recognizing receptor is integrated at a CD70 locus, and the second antigen-recognizing receptor is integrated at a TRAC locus. In certain embodiments, the gene disruption of the CD70 locus is produced by the integration of the first antigen-recognizing receptor at the CD70 locus. In certain embodiments, a CRISPR system is used to integrate the first antigen-recognizing receptor to the CD70 locus. In certain embodiments, a CRISPR system is used to integrate the second antigen-recognizing receptor to the TRAC locus.

In certain embodiments, the cell is a T cell, the first antigen-recognizing receptor is integrated at a TRAC locus, and the second antigen-recognizing receptor is integrated at a TRAC locus. In certain embodiments, a CRISPR system is used to integrate the first antigen-recognizing receptor to the TRAC locus. In certain embodiments, a CRISPR system is used to integrate the second antigen-recognizing receptor to the TRAC locus.

5.3.5. Gene Disruption of B2M Locus

In certain embodiments, a presently disclosed cell further comprises a gene disruption of a B2M locus. In certain embodiments, the gene disruption of the B2M locus results in a non-functional beta 2-microglobulin. In certain embodiments, the gene disruption of the B2M locus results in knockout of the B2M gene expression.

Any methods to generate the gene disruption of the CD70 locus as disclosed in Section 5.2 can be used to generate the gene disruption of the B2M locus. In certain embodiments, the gene disruption of the B2M locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the B2M locus can be a disruption of the coding region of the B2M locus and/or a disruption of the non-coding region of the B2M locus. In certain embodiments, the gene disruption of the B2M locus comprises a disruption of the coding region of the B2M locus. In certain embodiments, the gene disruption of the B2M locus comprises an insertion at the coding region of the B2M locus. Human B2M protein comprises four exons: exon 1, exon 2, exon 3, and exon 4. In certain embodiments, the coding region of the B2M locus comprises exon 1, exon 2, and exon 3. In certain embodiments, the gene disruption of the B2M locus comprises a disruption at one or both of exon 1 and exon 2 of the B2M locus. In certain embodiments, the gene disruption of the B2M locus comprises a disruption at exon 1 of the B2M locus. In certain embodiments, the gene disruption of the B2M locus comprises an insertion at exon 1 of the B2M locus.

Immune rejection of cells is due to the expression of human leucocyte antigen class I molecules (HLA-I) on the surface of these cells (Zhang et al., J Cell Mol Med. (2020); 24:695-710). HLA-I presents “non-self” antigens to CD8⁺ T cells that eliminate the transplanted cells through direct cytotoxic effect (Zhang 2020). Due to the polymorphic nature of the HLA-I genes, it is often difficult to identify a perfect match between donor and recipient prior to transplantation (Zhang 2020). HLA-I comprises a heavy chain and a light chain, which is also called β₂-microglobulin (B2M). HLA-I structure is disrupted and non-functional when the B2M gene is deleted (Zhang 2020). In certain embodiments, the gene disruption of the B2M locus can reduce cell-induced immune rejection, thereby making the cells more suitable for an allogeneic setting.

5.3.6. Gene Disruption of CIITA Locus

In certain embodiments, a presently disclosed cell further comprises a gene disruption of a Class II transactivator (CIITA) locus. In certain embodiments, the gene disruption of the CIITA locus results in a non-functional MHC class II transactivator. In certain embodiments, the gene disruption of the CIITA locus results in knockout of the CIITA gene expression.

Any methods to generate the gene disruption of the CD70 locus as disclosed in Section 5.2 and any methods to generate the gene disruption of a B2M locus as disclose din 5.3.5 can be used to generate the gene disruption of the CIITA locus. In certain embodiments, the gene disruption of the CIITA locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the CIITA locus can be a disruption of the coding region of the CIITA locus and/or a disruption of the non-coding region of the CIITA locus.

In certain embodiments, the gene disruption of the CIITA locus comprises a disruption of the coding region of the CIITA locus. In certain embodiments, the gene disruption of the CIITA locus comprises an insertion at the coding region of the CIITA locus. Human CIITA protein comprises 20 exons: exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, and exon 20. In certain embodiments, the coding region of the CIITA locus comprises exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, and exon 19. In certain embodiments, the gene disruption of the CIITA locus comprises a disruption at one or more of exon 1 through exon 19 of the CIITA locus. In certain embodiments, the gene disruption of the CIITA locus comprises a disruption at exon 3 of the CIITA locus. In certain embodiments, the gene disruption of the CIITA locus comprises an insertion at exon 3 of the CIITA locus.

CIITA is a transcriptional coactivator that regulates y-interferon-activated transcription of Major Histocompatibility Complex (MHC) class I and II genes (Devaiah et al., Frontiers in Immunology (2013); Vol. 4; Article 476:1-6). Thus, CIITA plays a critical role in immune responses: CIITA deficiency results in aberrant MHC gene expression and consequently in autoimmune diseases such as Type II bare lymphocyte syndrome (Devaiah 2013). Although CIITA does not bind to DNA directly, it regulates MHC transcription in two distinct ways—as a transcriptional activator and as a general transcription factor (Devaiah 2013). The CIITA is a master regulator of MHC gene expression (Devaiah 2013). CIITA induces de novo transcription of MHC class II genes and enhances constitutive MHC class I gene expression (Devaiah 2013). In certain embodiments, the gene disruption of the CIITA locus can reduce immune rejection, and improve survival of allogeneic immune cells, thereby making the cells more suitable for an allogeneic setting.

5.4. Nucleic Acid Compositions and Vectors

The presently disclosed subject matter provides nucleic acid compositions comprising a first polynucleotide encoding a first antigen-recognizing receptor disclosed herein (e.g., disclosed in Section 5.3.1) and a second polynucleotide encoding a second antigen-recognizing receptor disclosed herein (e.g., disclosed in Section 5.3.4). Also provided are cells comprising such nucleic acid compositions. In certain embodiments, the nucleic acid composition further comprises a first promoter that is operably linked to the first antigen-recognizing receptor. In certain embodiments, the nucleic acid composition further comprises a second promoter that is operably linked to the second antigen-recognizing receptor.

In addition, the presently disclosed subject matter provides nucleic acid compositions comprising a first polynucleotide encoding a first antigen-recognizing receptor disclosed herein (e.g., disclosed in Section 5.3.1) and a second polynucleotide encoding a fusion polypeptide disclosed herein (e.g., disclosed in Section 5.3.3). Also provided are cells comprising such nucleic acid compositions. In certain embodiments, the nucleic acid composition further comprises a first promoter that is operably linked to the fusion polypeptide. In certain embodiments, the nucleic acid composition further comprises a second promoter that is operably linked to the first antigen-recognizing receptor.

In certain embodiments, one or both of the first and second promoters are endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

In certain embodiments, the first and/or the second antigen-recognizing receptors are integrated at a locus within the genome of the T cell, e.g., a TRAC locus, a TRBC locus, a TRDC locus, or a TRGC locus. In certain embodiments, the locus is a TRAC locus. In certain embodiments, the expression of the first and/or second antigen-recognizing receptors are under the control of an endogenous promoter. Non-limiting examples of endogenous promoters include an endogenous TRAC promoter, an endogenous TRBC promoter, an endogenous TRDC promoter, and an endogenous TRGC promoter. In certain embodiments, the endogenous promoter is an endogenous TRAC promoter.

In certain embodiments, the nucleic acid composition is a vector. In certain embodiments, the vector is a retroviral vector (e.g., a gamma-retroviral vector or a lentiviral vector). In certain embodiments, the vector is viral vectors selected from the group consisting of adenoviral vectors, adena-associated viral vectors, vaccinia viruses, bovine papilloma viruses, and herpes viruses (e.g., such as Epstein-Barr Virus).

Additionally, the nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, the first polynucleotide and the second polynucleotide can be cloned into a retroviral vector and expression can be driven from its endogenous promoter, from the retroviral long terminal repeat, or from a promoter specific for a target cell type of interest. Non-viral vectors may be used as well.

The first polynucleotide and the second polynucleotide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be delivered into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

5.5. Formulations and Administration

The presently disclosed subject matter provides compositions comprising presently disclosed cells (e.g., disclosed in Section 5.3). In certain embodiments, the compositions are pharmaceutical compositions that further comprise a pharmaceutically acceptable excipient.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. In certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus). The cells can be introduced by injection, catheter, or the like.

Compositions comprising the presently disclosed cells can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, compositions, or nucleic acid compositions are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells) or NK cells) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

5.6. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor (or neoplasm) in a subject. The presently disclosed cells and compositions comprising thereof can be used for prolonging the survival of a subject suffering from a tumor. In certain embodiments, the tumor is cancer. The presently disclosed cells, compositions, and nucleic acid compositions can also be used for treating and/or preventing a pathogen infection or other infectious disease in a subject, such as an immunocompromised human subject. The presently disclosed cells, compositions, and nucleic acid compositions can also be used for treating and/or preventing an autoimmune disease in a subject. In certain embodiments, each of the above-noted method comprises administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of tumors (or neoplasms) include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, compositions, nucleic acid compositions can be used for treating and/or preventing blood cancers (e.g., leukemias, lymphomas, and myelomas) or ovarian cancer, which are not amenable to conventional therapeutic interventions.

In certain embodiments, the tumor and/or neoplasm is a solid tumor. Non limiting examples of solid tumor include renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the tumor and/or neoplasm is a blood cancer. Non-limiting examples of blood cancer include multiple myeloma, leukemia, and lymphomas. Non-limiting examples of leukemia include acute myeloid leukemia (AML), chronic myeloid leukemia (CMIL), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. The lymphoma can be Hodgkin's lymphoma or non-Hodgkin's lymphoma. In certain embodiments, the lymphoma is non-Hodgkin's lymphoma, including B-cell non-Hodgkin's lymphoma and T-cell non-Hodgkin's lymphoma.

In certain embodiments, the tumor and/or neoplasm is a B cell malignancy. Non-limiting examples of B cell malignancy include B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments, the tumor and/or neoplasm is a B cell-related neoplasm. Non-limiting examples of B cell-related neoplasm include chronic lymphocytic leukemia/small lymphocytic lymphoma (CLL/SLL), B-cell prolymphocytic leukemia, splenic marginal zone lymphoma, hairy cell leukemia, splenic B-cell lymphoma/leukemia (unclassifiable), splenic diffuse red pulp small B-cell lymphoma, lymphoplasmacytic lymphoma, Waldenstrom macroglobulinemia, monoclonal gammopathy of undetermined significance (MGUS, IgM), heavy-chain diseases (μ, γ, α), MGUS (IgG/A), plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with IRF4 rearrangement, primary cutaneous follicle center cell lymphoma, mantle cell lymphoma, in situ mantle cell neoplasia, diffuse large B-cell lymphoma (DLBCL) (not otherwise specified (NOS)), germinal center B-cell type, activated B-cell type, T-cell/histiocyte-rich large B-cell lymphoma, primary DLBCL of the central nervous system (CNS), primary cutaneous DLBCL (leg type), Epstein-Barr virus (EBV)-positive DLBCL (NOS), EBV-positive mucocutaneous ulcer, DLBCL associated with chronic inflammation, lymphomatoid granulomatosis, primary mediastinal (thymic) large B-cell lymphoma, intravascular large B-cell lymphoma, anaplastic lymphoma kinase (ALK)-positive large B-cell lymphoma, plasmablastic lymphoma, primary effusion lymphoma, human herpesvirus 8 (HHV-8)-associated DLBCL (NOS), Burkitt lymphoma, Burkitt-like lymphoma with 11q aberration, high-grade B-cell lymphoma with MYC and BLC2 and/or BCL6 rearrangements, high-grade B-cell lymphoma (NOS), and B-cell lymphoma (unclassifiable).

In certain embodiments, the tumor and/or neoplasm is a myeloid disorder. Non-limiting examples of myeloid disorders include myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

In certain embodiments, the myeloid disorder is acute myeloid leukemia (AML). In certain embodiments, the first and/or second antigens are independently selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHCC11.

In certain embodiments, the myeloid disorder is acute myeloid leukemia (AML), and the first and/or second antigens are expressed on an AML hematopoietic stem/progenitor cell (HSPC) and/or a leukemia stem cell (LSC). In certain embodiments, the AML HSPC expresses CD34. In certain embodiments, the first antigen and/or the second antigen are not expressed or expressed at a non-detectable level in a non-hematopoietic stem cell and/or a non-hematopoietic progenitor cell. In certain embodiments, the first antigen and/or the second antigen are independently selected from the group consisting of CD70, IL1RAP, CD33, CLEC12A, ADGRE2, CD123 and combinations thereof. In certain embodiments, the first antigen is CD70. In certain embodiments, the first antigen and the second antigen are CD70 and IL1RAP.

In certain embodiments, the method further comprises administering to the subject a CD70-targeted therapy. In certain embodiments, the subject receives a CD70-targeted therapy. In certain embodiments, the CD70-targeted therapy is administered prior to, simultaneously, or post the administration of the presently disclosed cells or compositions comprising thereof. In certain embodiments, the CD70-targeted therapy comprises an anti-CD70 antibody (e.g., monoclonal antibodies (mAbs), CD70-targeted antibody-drug conjugates (ADC), CD70-targeted multispecific or bispecific molecules, e.g. bispecific T cell engagers (BiTE), checkpoint-inhibitory T cell-engagers (CiTE), simultaneous multiple interaction T cell engagers (SMiTE), dual affinity retargeting bispecific molecules (DART), tetravalent tandem diabodies (TandAb), Triomab, kih IgG common LC, ortho-Fab IgG, DVD-Ig, 2 in 1-IgG, IgG-scFv, scFv2-Fc, bi-Nanobody, DART-Fc, scFv-HAS-scFv, DNL-Fab3, CrossMAb asymmetric IgG-based bispecific T cell-engaging or trispecific killer engagers (TriKEs), CD70-targeted-BiTE-secreting CAR T cells (CART.BiTE cells), and CAR-T cells secreting one or more CD70-targeting molecules.

The presently disclosed subject matter provides methods for treating and/or preventing a viral infection in a subject. The method can comprise administering an effective amount of the presently disclosed cells, a presently disclosed composition, or a presently disclosed nucleic acid composition to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

The presently disclosed subject matter provides methods for treating and/or preventing a bacterial infection in a subject. The method can comprise administering an effective amount of the presently disclosed cells, a presently disclosed composition, or a presently disclosed nucleic acid composition to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella, shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an autoimmune disease.

The presently disclosed subject matter provides methods for treating and/or preventing an infectious disease in a subject. The method can comprise administering an effective amount of the presently disclosed cells, a presently disclosed composition, or a presently disclosed nucleic acid composition to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed antigen-recognizing receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed antigen-recognizing receptor. The incorporation of a suicide gene into the a presently disclosed antigen-recognizing receptor gives an added level of safety with the ability to eliminate the majority of receptor-expressing cells within a very short time period. A presently disclosed cell incorporated with a suicide gene can be pre-emptively eliminated at a given timepoint post the cell infusion, or eradicated at the earliest signs of toxicity.

5.7. Kits

The presently disclosed subject matter provides kits for inducing and/or enhancing an immune response and/or treating and/or preventing a neoplasm or a pathogen infection (e.g., an autoimmune disease or an infectious disease) in a subject. In certain embodiments, the kit comprises an effective amount of presently disclosed cells, a presently disclosed composition, or a presently disclosed nucleic acid composition. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR, a TCR, or a TCR like fusion molecule) directed toward an antigen of interest in expressible form, which may optionally be comprised in the same or different vectors.

If desired, the cells, composition, or nucleic acid composition are provided together with instructions for administering the cells, composition, or nucleic acid composition to a subject having or at risk of developing a tumor (e.g., a cancer) or a pathogen infection (e.g., an infectious disease), or immune disorder (e.g., an autoimmune disease). The instructions generally include information about the use of the cell, composition or nucleic acid composition for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

Summary

The inventors previously determined that knocking-in the cDNA encoding a CAR or a TCR into the TRAC locus generates markedly superior T cells compared to T cells generated by g-retroviral or lentiviral transduction methods. The need to engineer multiple receptors for antigen into single cells (to address tumor heterogeneity) or additional genes of therapeutic interest (e.g. for “armored” T cells) creates the need for multiplexed editing. Furthermore, endogenous genes may be simultaneously knocked-out in the same cell, e.g., to enhance CAR T cell function and/or prevent immune rejection of CAR T cells in allogeneic recipients (e.g., by knocking out the B2M gene (encodes β2-microglobulin)). The inventors studied various knock-in (KI)/knock-out (KO) “scenarios” of clinical relevance in a hematological model, by analyzing T cell potency and editing safety (off-target effects and chromosomal translocations). The multiplexed editing is critical for both therapeutic efficacy and safety in clinical protocols.

Technology

The inventors previously demonstrated that gene editing using the CRISPR/Cas9 technology allows targeted integration of the CAR cDNA into the TRAC locus, thereby placing CAR expression under the control of the TCR promoter, which provides optimal regulation of CAR expression and better tumor control when compared to current gene transfer methods (WO2017180989 and Eyquem et al., Nature. (2017 Mar. 2); 543(7643): 113-117). It is unequivocal that precision engineering can enhance the use of CARs, TCR, CCRs and HITs, as well as that of other therapeutic genes that also ought to be expressed in regulated fashion in CAR T cells (cytokines, chemokines, scFv's, Bites, etc).

Multiplexed gene editing thus becomes a platform to advance immune cell-based therapies. Multiplexed CRISPR/Cas9-based gene editing consisting of 3 knockouts combined with a lentiviral vector transduction was recently tested in three subjects (Stadtmauer et al., Science (2020), volume 367, issue 6481, eaba7365), which highlighted the need to improve editing conditions and minimize translocations. In order to establish the multiple editing components in a controlled fashion, the inventors investigated three scenarios of different complexity regarding the number of targeted integration sites and additional gene KO sites in acute myeloid leukemia (AML)
Scenario I: 1 KI and 2 KO (2 receptors expressed at same gene locus, under control of the same promoter, and 1 additional KO)
Scenario II: 2 KI and 2 KO (2 receptors expressed at separate gene loci, under control of different promoters, no additional KO)
Scenario III: Either 1 or 2 KI (whatever proves to be superior) and accordingly 2 or 1 additional KO

Results

The AML targets the inventors investigated were CD70 and IL1RAP. The inventors previously identified these two cell surface molecules to possess attractive expression features due to their presence in leukemic stem cells and absence or very low-level expression in hematopoietic stem and progenitor cells (Perna et al, Cancer cell 32.4 (2017): 506-519).). FIGS. 1A and 1B show that CD70 was highly expressed on a substantial fraction of CAR T cells, which may lead to T cell fratricide/suicide and impair CAR efficacy.

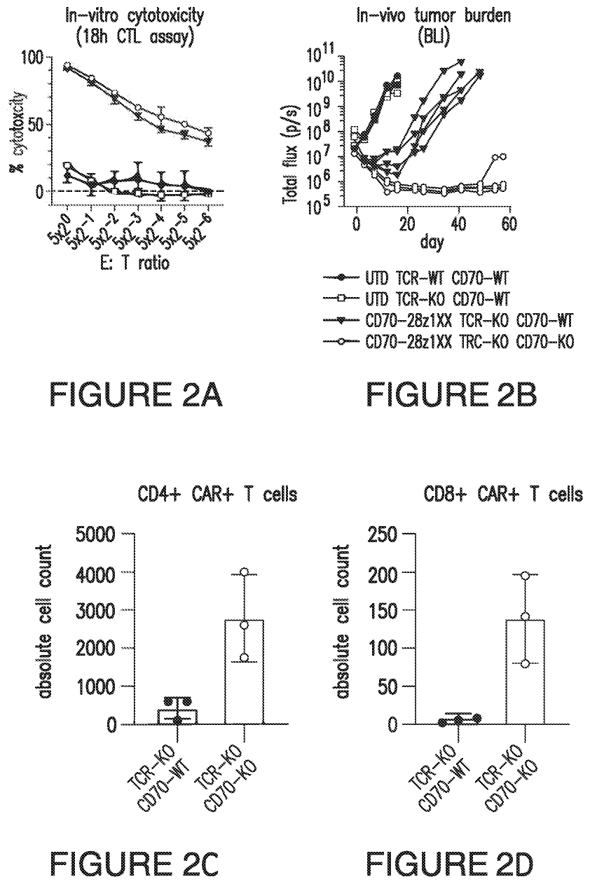

A highly efficient CRISPR/Cas9-based knockout (KO) of CD70 in T cells was established prior to CD70-CAR expression, which allowed complete tumor control and increased CAR T cell numbers in our in vivo model (see FIGS. 2A-2D). As shown in FIGS. 2A-2D, knocking-out CD70 improved the efficacy of CD70-CAR T cells, which translated in reduced tumor burden and concomitant increase of CD4⁺ and CD8⁺ CAR T cell numbers.

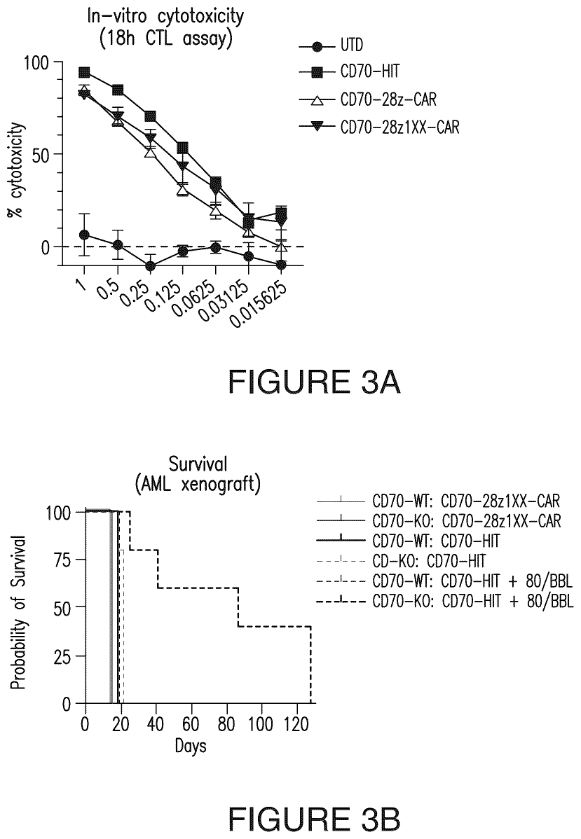

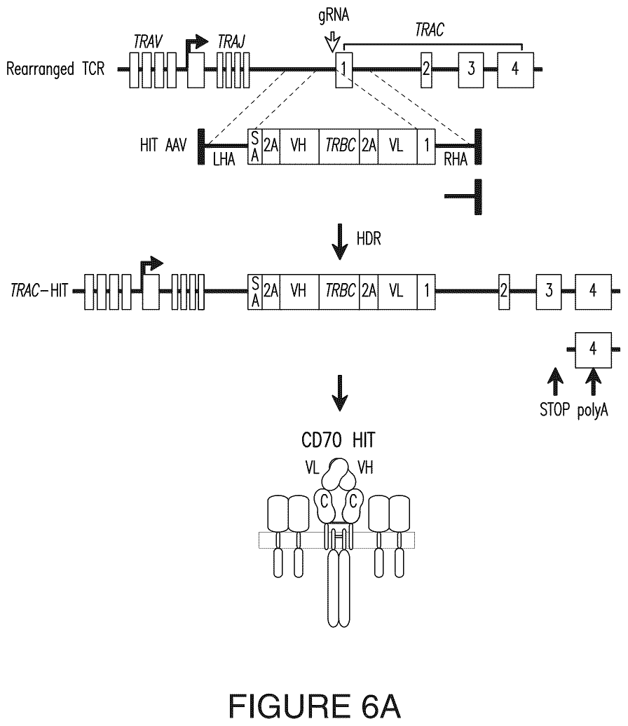

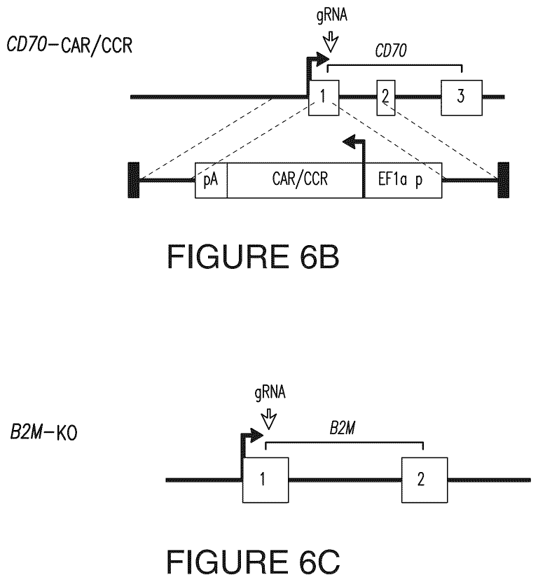

As CD70 is expressed at very low levels in AML (leading to failure of conventional second-generation CAR constructs), a TCR-like HIT CAR targeting CD70 as previously disclosed in International Patent Publication No.: WO2019157454A1 was generated to eliminate leukemia cells with very low CD70 expression. The CD70-HIT was integrated to a TRAC locus as shown in FIG. 6A. By knocking-out CD70 and providing additional co-stimulation, CD70-HIT allowed efficient reduction of AML burden leading to a significant survival benefit while a second-generation CAR failed to control leukemic growth (see FIGS. 3A and 3B). As shown in FIG. 3B, CD70-HIT with CD70 knockout in vivo efficiently reduced leukemic growth leading to long-term survival.

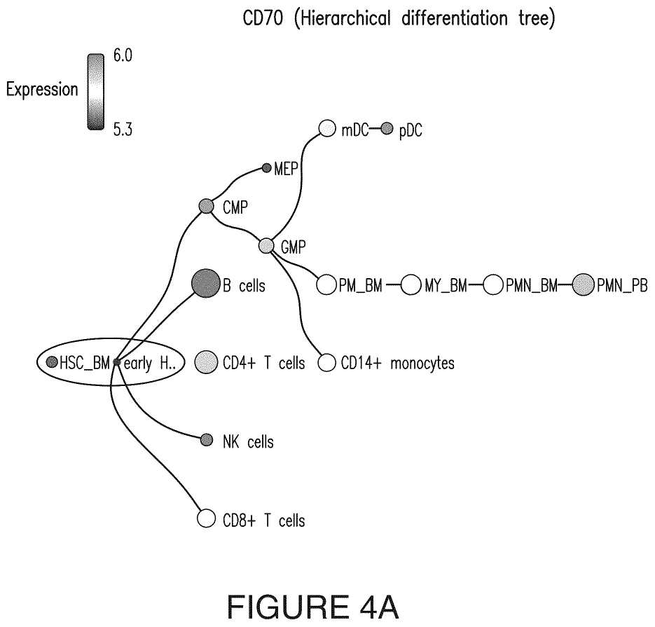

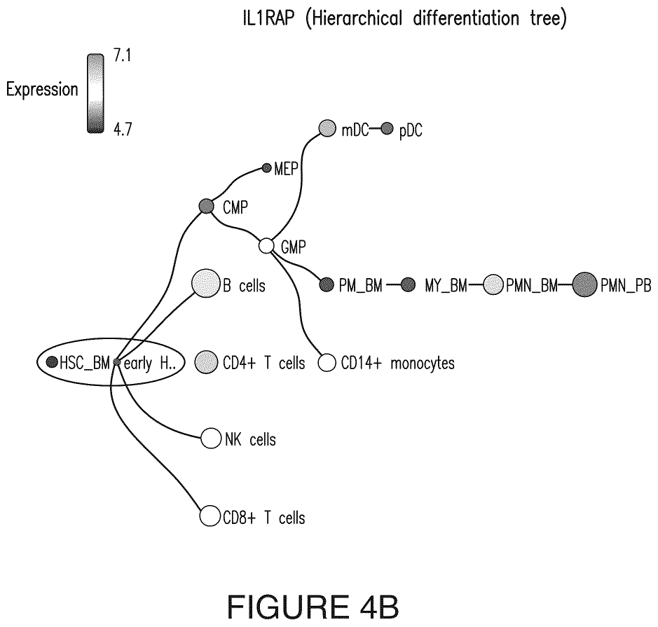

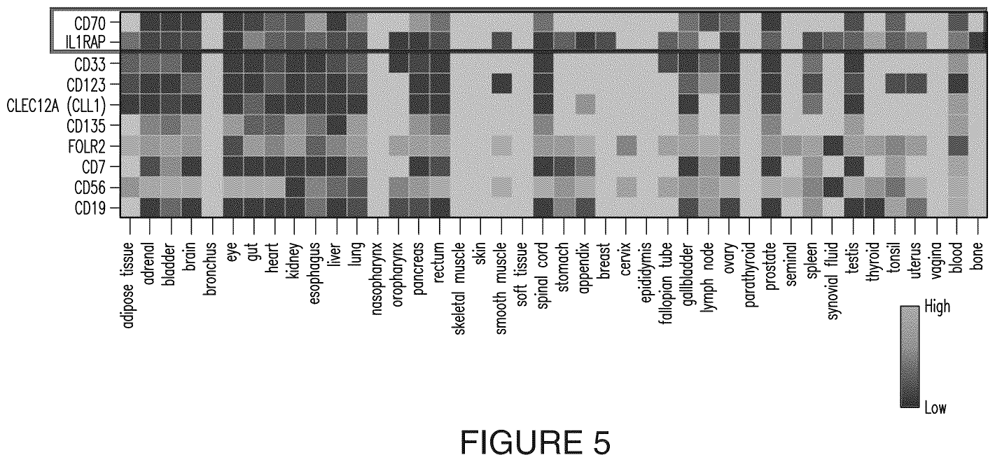

In order to further enhance specific elimination of leukemic stem cells (LSC), which have been reported to cause relapse in AML, IL1RAP was chosen as a second target as it has been shown that IL1RAP expression contributes to clonogenicity of LSC and correlates with poor patient survival (Barreyro et al. Blood (2012); 120(6):1290-8). The expressions of CD70 and IL1RAP were assessed in normal hematopoiesis and non-hematopoietic normal tissues. The data shown in FIGS. 4A, 4B, and 5 demonstrate a favorable combinatorial profile of both CD70 and IL1RAP, suggesting that co-targeting may spare HSPC and other vital normal tissues, allowing for both OR-gated and AND-gated combinatorial targeting approaches. A second knock-in (e.g., a CAR or a CCR expression cassette) is introduced to exon 1 of the first exon of the CD70 gene as shown in FIG. 6B.

An additionally knock-out of B2M (scenario III) is introduced to the cell in order to provide options for potential applications in an allogeneic setting. FIG. 6C depicts gene-editing strategy to generate B2M-KO. CRISPR/Cas9 can be targeted to the first exon of the B2M gene (as shown in Eyquem et al., Nature (2017); 543:113-117).

The combinations that were and are investigated are shown in Table 2.

TABLE 2

| | | | 2^{nd} | | |
|---|---|---|---|---|---|
| | | | antigen- | | |
| Combi- | | | recog- | | Tar- |
| nation | 1^{st} antigen-recog- | KI | nizing | KI | geted |
| No. | nizing receptor | locus | receptor | locus | KO loci |
| 1 | CAR/HIT (e.g., CD70 | TRAC | X | X | TRAC, |
| | HIT or CD70 CAR) | | | | CD70 |
| 2 | CAR/HIT (e.g., CD70 | TRAC | CAR/CCR | TRAC | TRAC, |
| | HIT or CD70 CAR) | | | | CD70 |
| 3 | CAR/HIT (e.g., CD70 | TRAC | CAR/CCR | CD70 | TRAC, |
| | HIT or CD70 CAR) | | | | CD70 |
| 4 | CAR/HIT (e.g., CD70 | CD70 | CAR/CCR | TRAC | TRAC, |
| | HIT or CD70 CAR) | | | | CD70 |
| 5 | CAR/HIT (e.g., CD70 | TRAC | X | X | TRAC, |
| | HIT or CD70 CAR) | | | | CD70, |
| | | | | | B2M |
| 6 | CAR/HIT (e.g., CD70 | TRAC | CAR/CCR | TRAC | TRAC, |
| | HIT or CD70 CAR) | | | | CD70, |
| | | | | | B2M |
| 7 | CAR/HIT (e.g., CD70 | TRAC | CAR/CCR | CD70 | TRAC, |
| | HIT or CD70 CAR) | | | | CD70, |
| | | | | | B2M |
| 8 | CAR/HIT (e.g., CD70 | CD70 | CAR/CCR | TRAC | TRAC, |
| | HIT or CD70 CAR) | | | | CD70, |
| | | | | | B2M |
| 9 | CAR/HIT (e.g., CD70 | TRBC | X | X | TRBC, |
| | HIT or CD70 CAR) | | | | CD70 |
| 10 | CAR/HIT (e.g., CD70 | TRBC | CAR/CCR | TRBC | TRBC, |
| | HIT or CD70 CAR) | | | | CD70 |
| 11 | CAR/HIT (e.g., CD70 | TRBC | CAR/CCR | CD70 | TRBC, |
| | HIT or CD70 CAR) | | | | CD70 |
| 12 | CAR/HIT (e.g., CD70 | CD70 | CAR/CCR | TRBC | TRBC, |
| | HIT or CD70 CAR) | | | | CD70 |
| 13 | CAR/HIT (e.g., CD70 | TRBC | X | X | TRBC, |
| | HIT or CD70 CAR) | | | | CD70, |
| | | | | | B2M |
| 14 | CAR/HIT (e.g., CD70 | TRBC | CAR/CCR | TRBC | TRBC, |
| | HIT or CD70 CAR) | | | | CD70, |
| | | | | | B2M |
| 15 | CAR/HIT (e.g., CD70 | TRBC | CAR/CCR | CD70 | TRBC, |
| | HIT or CD70 CAR) | | | | CD70, |
| | | | | | B2M |
| 16 | CAR/HIT (e.g., CD70 | CD70 | CAR/CCR | TRBC | TRBC, |
| | HIT or CD70 CAR) | | | | CD70, |
| | | | | | B2M |

### Example 2

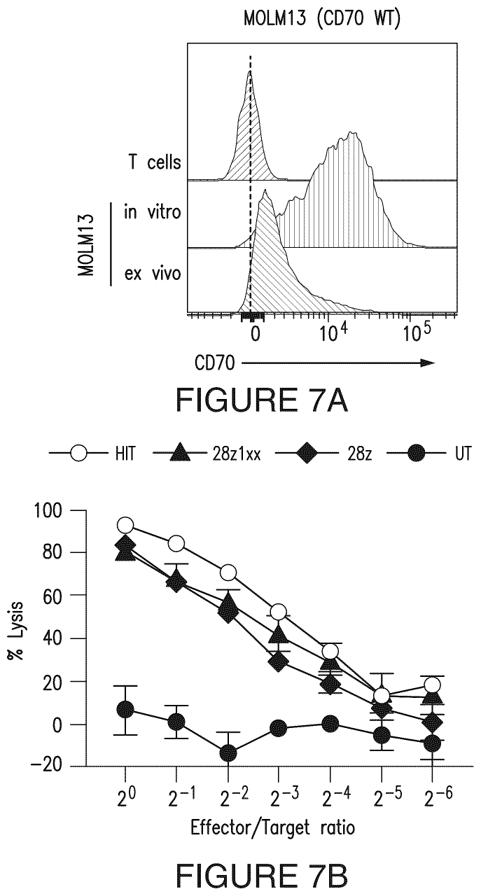

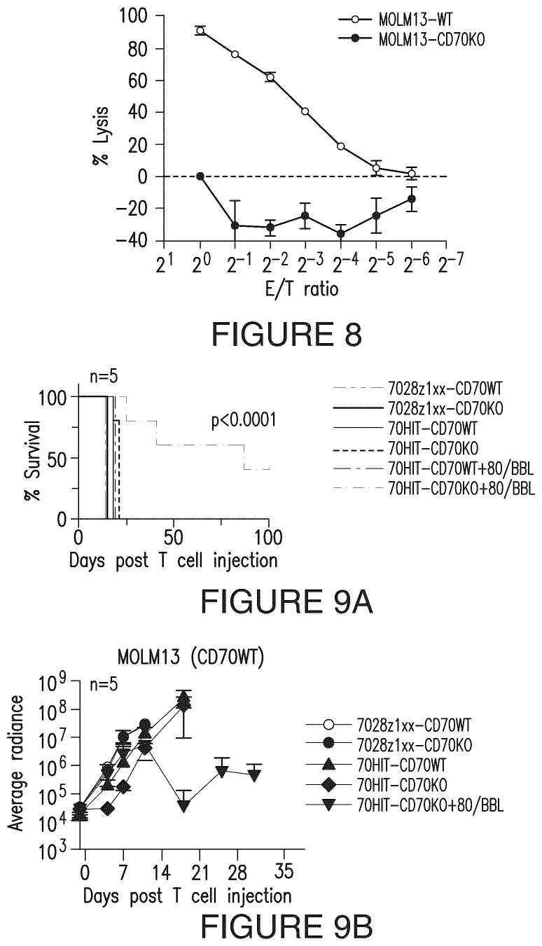

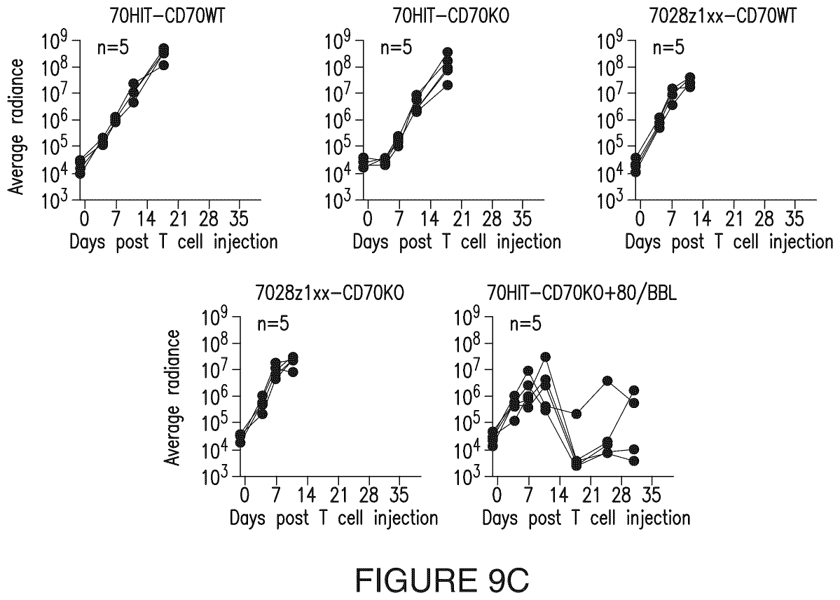

The capacity of TRAC-HIT T cells to control tumors expressing different antigen levels in vivo was studied using the AML cell line MOLM13 which was derived from FLT3-ITD relapsed AML and expresses a reduced number of CD70 molecules per cell based on ex vivo bone marrow analysis compared to in vitro cultured MOLM13 cells (see FIG. 7A). CD70-HIT and CD70-CAR T cells effectively lysed CD70+ MOLM13 in vitro (see FIG. 7B) but not CD70-target cells (see FIG. 8). However, neither CAR nor HIT T cells were effective in vivo (see FIGS. 9A-9C). CD70 expression was induced in activated T cells and high CD70 levels were detectable in a sizable fraction of CAR-transduced T cells (see FIG. 1), which result in HIT/CAR-mediated fratricide or suicide T cell killing and/or HIT/CAR malfunction when targeting CD70. Therefore, CD70 expression was ablated (see FIG. 1A) and it was found that anti-AML efficacy was improved. In this instance, CD70-HIT+80/BBL T cells vastly outperformed the CAR T cells (p<0.0001, see FIGS. 9B and 9C).

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A cell comprising:
a) a first antigen-recognizing receptor that targets a first antigen and is integrated at a locus within the genome of the T cell; and
b) a gene disruption of a CD70 locus.

2. The cell of claim 1, wherein the gene disruption comprises a substitution, a deletion, an insertion, or a combination thereof.

3. The cell of claim 1, wherein the gene disruption of the CD70 locus results in a non-functional CD70 protein or in knockout of the CD70 gene expression.

4. The cell of claim 1, wherein the cell is a cell of the lymphoid lineage or a cell of the myeloid lineage.

5. The cell of claim 4, wherein the cell of the lymphoid lineage is selected from the group consisting of T cells, B cells, Natural Killer (NK) cells, and dendritic cells.

6. The cell of claim 5, wherein the cell is a T cell.

7. The cell of claim 11, wherein the T cell is
a) a T cell derived from an induced pluripotent stem cell;
b) a CD8⁺ T cell;
c) a CD8⁺ T cell is CD4 independent; or
d) selected from the group consisting of a cytotoxic T lymphocyte (CTL), a γδ T cell, a tumor-infiltrating lymphocyte (TIL), a regulatory T cell, and a Natural Killer T (NKT) cell.

8. The cell of claim 1, wherein the locus is selected from the group consisting of a TRAC locus, a TRBC locus, a TRDC locus, and a TRGC locus.

9. The cell of claim 1, wherein the cell further comprises a second antigen-recognizing receptor that targets a second antigen.

10. The cell of claim 9, wherein the first antigen and/or the second antigen is a tumor antigen selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHCC11.

11. The cell of claim 1, wherein the first antigen is CD19 or CD70.

12. The cell of claim 9, wherein the first antigen and/or the second antigen is expressed in an acute myeloid leukemia (AML) hematopoietic stem/progenitor cell (HSPC) and/or a leukemia stem cell (LSC).

13. The cell of claim 12, wherein the AML HSPC expresses CD34 and/or the AML LSC expresses CD34.

14. The cell of claim 12, wherein the first antigen and/or the second antigen is not expressed or expressed at a non-detectable level in a non-malignant hematopoietic stem cell and/or a non-malignant hematopoietic progenitor cell.

15. The cell of claim 9, wherein the first antigen and/or the second antigen is independently selected from the group consisting of CD70, IL1RAP, CD19, CD33, CLEC12A, ADGRE2, CD123 and combinations thereof.

16. The cell of claim 15, wherein the first antigen and the second antigen are CD70 and IL1RAP.

17. The cell of claim 15, wherein the first antigen is CD70.

18. The cell of claim 1, wherein the first antigen-recognizing receptor is a chimeric receptor, a T cell receptor (TCR), a TCR like fusion molecule.

19. The cell of claim 18, wherein the first antigen-recognizing receptor is a TCR like fusion molecule.

20. The cell of claim 18, wherein the chimeric receptor is a chimeric antigen receptor (CAR).

21. The cell of claim 9, wherein the second antigen-recognizing receptor is a chimeric antigen receptor (CAR) or a chimeric co-stimulating receptor (CCR).

22. The cell of claim 21, wherein the CAR comprises an extracellular antigen-binding domain that binds to the second antigen, and an intracellular signaling domain that is capable of delivering an activation signal to the cell.

23. The cell of claim 9, wherein
a) the cell is a T cell, and the first antigen-recognizing receptor and the second antigen-recognizing receptor are integrated at a TRAC locus of the T cell;
b) the cell is a T cell, the first antigen-recognizing receptor is integrated at a TRAC locus of the T cell, and the second antigen-recognizing receptor is integrated at the CD70 locus;
c) the cell is a T cell, the first antigen-recognizing receptor is integrated at the CD70 locus, and the second antigen-recognizing receptor is integrated at a TRAC locus of the T cell;
d) the cell is a T cell, and the first antigen-recognizing receptor and the second antigen-recognizing receptor are integrated at a TRBC locus of the T cell;
e) the cell is a T cell, the first antigen-recognizing receptor is integrated at a TRBC locus of the T cell, and the second antigen-recognizing receptor is integrated at the CD70 locus; and/or
f) the cell is a T cell, the first antigen-recognizing receptor is integrated at the CD70 locus, and the second antigen-recognizing receptor is integrated at a TRBC locus of the T cell.

24. The cell of claim 1, wherein the cell further comprises at least one exogenous costimulatory ligand.

25. The cell of claim 24, wherein the at least one exogenous a costimulatory ligand comprises CD80 and/or 4-1BBL.

26. The cell of claim 1, wherein the cell further comprises a fusion polypeptide comprising: a) an extracellular domain and a transmembrane domain of a co-stimulatory ligand, and b) an intracellular domain of a first co-stimulatory molecule.

27. The cell of claim 26, wherein the co-stimulatory ligand is CD80 and the first co-stimulatory molecule is 4-1BB.

28. The cell of claim 1, wherein the cell further comprises a gene disruption of a TRBC locus, a B2M locus, and/or a CIITA locus.

29. The cell of claim 1, wherein the gene disruption of the CD70 locus is capable of enhancing at least one activity of the cell comprising the first antigen-recognizing receptor, wherein the at least one activity comprises cytotoxicity, cell proliferation, cell persistence, or a combination thereof.

30. The cell of claim 1, wherein the cell is autologous or allogeneic.

31. A composition comprising the cell of claim 1.

32. A method for producing a cell of claim 1, the method comprising generating a gene disruption of a CD70 locus in a cell comprising the first antigen-recognizing receptor.

33. A cell produced by the method of claim 32.

34. A method of reducing tumor burden in a subject, preventing and/or treating a neoplasm or a tumor in the subject, the method comprising administering to the subject an effective amount of the cells of claim 1.

35. The method of claim 34, wherein the method reduces the number of tumor cells, reduces tumor size, and/or eradicates the tumor in the subject.

36. The method of claim 34, wherein the neoplasm or tumor is a myeloid disorder.

37. The method of claim 36, wherein the myeloid disorder is selected from the group consisting of myelodysplastic syndromes, myeloproliferative neoplasms, chronic myelomonocytic leukemia, or acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

38. The method of claim 37, wherein the myeloid disorder is acute myeloid leukemia (AML).

39. A method of preventing and/or treating a pathogen infection in a subject, preventing and/or treating an autoimmune disease in a subject, and/or preventing and/or treating an infectious disease in a subject, the method comprising administering to the subject an effective amount of the cells of any one of claim 1.

40. The method of claim 34, further comprising administering to the subject a CD70-targeted therapy.

41. The method of claim 34, wherein the subject receives a CD70-targeted therapy.

42. A kit comprising the cell of claim 1.


SEQUENCE LISTING (82 sequences)

SEQ ID NO: 1 - protein, 13 aa, synthetic construct
  REGION 1..13: note = CDR1
AASGFTFSSYIMH

SEQ ID NO: 2 - protein, 10 aa, synthetic construct
  REGION 1..10: note = Description of sequence: antibody component
VISYDGRNKY

SEQ ID NO: 3 - protein, 11 aa, synthetic construct
  REGION 1..11: note = Description of sequence: antibody component
ARDTDGYDFDY

SEQ ID NO: 4 - protein, 11 aa, synthetic construct
  REGION 1..11: note = Description of sequence: antibody component
RASQSVSSYLA

SEQ ID NO: 5 - protein, 8 aa, synthetic construct
  REGION 1..8: note = Description of sequence: antibody component
YDASNRAT

SEQ ID NO: 6 - protein, 9 aa, synthetic construct
  REGION 1..9: note = Description of sequence: antibody component
QQRTNWPLT

SEQ ID NO: 7 - protein, 118 aa, synthetic construct
  REGION 1..118: note = Description of sequence: antibody component
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSS

SEQ ID NO: 8 - protein, 107 aa, synthetic construct
  REGION 1..107: note = Description of sequence: antibody component
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 9 - protein, 240 aa, synthetic construct
  REGION 1..240: note = Description of sequence: antibody component
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 10 - protein, 15 aa, synthetic construct
  REGION 1..15: note = Description of sequence: antibody component
GGGGSGGGGSGGGGS

SEQ ID NO: 11 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 12 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - protein, 112 aa, synthetic construct
  REGION 1..112: note = Description of sequence: antibody component
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 22 aa, synthetic construct
  REGION 1..22: note = Description of sequence: antibody component
QNQLYNELNLGRREEYDVLDKR

SEQ ID NO: 15 - DNA, 66 bp, synthetic construct
  misc_feature 1..66: note = Description of sequence: DNA encoding antibody component
  source 1..66: mol_type = other DNA
cagaaccagctctataacgagctcaatctaggacgaagagaggagtacgatgttttggacaagaga

SEQ ID NO: 16 - protein, 22 aa, synthetic construct
  REGION 1..22: note = Description of sequence: antibody component
QNQLFNELNLGRREEFDVLDKR

SEQ ID NO: 17 - DNA, 66 bp, synthetic construct
  misc_feature 1..66: note = Description of sequence: DNA encoding antibody component
  source 1..66: mol_type = other DNA
cagaaccagctctttaacgagctcaatctaggacgaagagaggagttcgatgttttggacaagaga

SEQ ID NO: 18 - protein, 23 aa, synthetic construct
  REGION 1..23: note = Description of sequence: antibody component
QEGLYNELQKDKMAEAYSEIGMK

SEQ ID NO: 19 - DNA, 69 bp, synthetic construct
  misc_feature 1..69: note = Description of sequence: DNA encoding antibody component
  source 1..69: mol_type = other DNA
caggaaggcctgtacaatgaactgcagaaagataagatggcggaggcctacagtgagattgggatgaaa

SEQ ID NO: 20 - protein, 23 aa, synthetic construct
  REGION 1..23: note = Description of sequence: antibody component
QEGLFNELQKDKMAEAFSEIGMK

SEQ ID NO: 21 - DNA, 69 bp, synthetic construct
  misc_feature 1..69: note = Description of sequence: DNA encoding antibody component
  source 1..69: mol_type = other DNA
caggaaggcctgttcaatgaactgcagaaagataagatggcggaggccttcagtgagattgggatgaaa

SEQ ID NO: 22 - protein, 22 aa, synthetic construct
  REGION 1..22: note = Description of sequence: antibody component
HDGLYQGLSTATKDTYDALHMQ

SEQ ID NO: 23 - DNA, 66 bp, synthetic construct
  misc_feature 1..66: note = Description of sequence: DNA encoding antibody component
  source 1..66: mol_type = other DNA
cacgatggcctttaccagggtctcagtacagccaccaaggacacctacgacgcccttcacatgcag

SEQ ID NO: 24 - protein, 22 aa, synthetic construct
  REGION 1..22: note = Description of sequence: antibody component
HDGLFQGLSTATKDTFDALHMQ

SEQ ID NO: 25 - DNA, 66 bp, synthetic construct
  misc_feature 1..66: note = Description of sequence: DNA encoding antibody component
  source 1..66: mol_type = other DNA
cacgatggccttttccaggggctcagtacagccaccaaggacaccttcgacgcccttcacatgcag

SEQ ID NO: 26 - protein, 112 aa, synthetic construct
  REGION 1..112: note = Description of sequence: antibody component
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 27 - DNA, 336 bp, synthetic construct
  misc_feature 1..336: note = Description of sequence: DNA encoding antibody component
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 28 - DNA, 123 bp, synthetic construct
  misc_feature 1..123: note = Description of sequence: DNA encoding antibody component
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 29 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 30 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 31 - protein, 462 aa, synthetic construct
  REGION 1..462: note = Description of sequence: antibody component
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 32 - DNA, 1386 bp, synthetic construct
  misc_feature 1..1386: note = Description of sequence: DNA encoding antibody component
  source 1..1386: mol_type = other DNA
caggtgcagctggtggagtctgggggaggcgtggtccagcctgggaggtccctgagactctcctgtgcagcctctggatttaccttcagtagctatattatgcactgggtccgccaggctccaggcaaggggctggagtgggtggcagttatatcatatgatggaagaaacaaatactacgcagactccgtgaagggccgattcaccatctccagagacaattccaagaacacgctgtatctgcaaatgaacagcctgagagctgaggacacggctgtgtattactgtgcgagagatacggatggctacgattttgactactggggccagggaaccctggtcaccgtctcctcaggtggaggtggatcaggtggaggtggatctggtggaggtggatctgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaccaactggccgctcactttcggcggagggaccaaggtggagatcaaagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 33 - protein, 20 aa, synthetic construct
  REGION 1..20: note = Description of sequence: antibody component
MYRMQLLSCIALSLALVTNS

SEQ ID NO: 34 - protein, 20 aa, synthetic construct
  REGION 1..20: note = Description of sequence: antibody component
MYSMQLASCVTLTLVLLVNS

SEQ ID NO: 35 - protein, 20 aa, synthetic construct
  REGION 1..20: note = Description of sequence: antibody component
METPAQLLFLLLLWLPDTTG

SEQ ID NO: 36 - protein, 20 aa, synthetic construct
  REGION 1..20: note = Description of sequence: antibody component
METDTLLLWVLLLWVPGSTG

SEQ ID NO: 37 - protein, 21 aa, synthetic construct
  REGION 1..21: note = Description of sequence: antibody component
MALPVTALLLPLALLLHAARP

SEQ ID NO: 38 - protein, 18 aa, synthetic construct
  REGION 1..18: note = Description of sequence: antibody component
MALPVTALLLPLALLLHA

SEQ ID NO: 39 - protein, 16 aa, synthetic construct
  REGION 1..16: note = Description of sequence: antibody component
MKWVTFISLLFSSAYS

SEQ ID NO: 40 - protein, 30 aa, synthetic construct
  REGION 1..30: note = Description of sequence: antibody component
MDSKGSSQKGSRLLLLLVVSNLLLCQGVVS

SEQ ID NO: 41 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - DNA, 4627 bp, Homo sapiens
  source 1..4627: mol_type = genomic DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 43 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 44 - protein, 175 aa, Homo sapiens
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 45 - DNA, 1448 bp, Homo sapiens
  source 1..1448: mol_type = genomic DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 46 - DNA, 1489 bp, Homo sapiens
  source 1..1489: mol_type = genomic DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 47 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 48 - protein, 189 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 49 - DNA, 5591 bp, Homo sapiens
  source 1..5591: mol_type = genomic DNA
ataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacaggtatgtgtttacgcatatcatctgtcagaacacttctttgaaagtgaatgctgcattttttcctttcagtattaatgaaaaacaaacataaatctttcttaaatattgttacatttaatggtagcataaatgccctgctacttttctatagaattaaaatggtataggttttggagaaaacaaaattgaaaaagttactgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaatgatgtttatctgttgttgacagaatgatgtttatctaaaaacagttccaattttctatttcctttgctgagacacaaaggggaggcaaatgtgcaaagcttgagggtagtcttaccactgtgcttaagtgttctgatttttctagtgatcagggcaaaataaaaagtatagtaagttccaaggcagtgaatattatacaggagagaagttacagttttataatgtgttttcctttacactaaattctaaaagtaaaaagtctttttttttttttgacagagtttcactcttgttgcccaagcaggtgtgctatggtatgatctcagctcactgcaacctccacctcccgggttcaagtgattctcttacttcagcctcccgacaggctgggattgcaggcgcctgccaccacacctggctaatttttgtgtttttagtagagatggggtttcaccatgttggccaggctggtctcaaattcctgacctcaagtgatccatccacctcggcctccaagtgctgggattatgggcgtcagccactgtgcccagcctaaaagtaaaatgtctttcatgagcttcccaaggcagctacgttaaggaggacacttctcttaatgtcattctacagtagatttctaatgctctttcttggaagtttgtttttctgagaaaagctaaaaatataacatggaagtgatcatattatataatcaatgaagtgcttttcaaggagataaaactaatctggtccacacttgcaaccaaccttgattgagagagagagagaactcaggatacacttgaagattttattatggggaacagttactttattctttttacctcaatcaatgcatggaaataagtgatagtcattttcatttatcttttaataaatgaagtcaccatgaggaaaataaaaagacattgaaaacccattaaagtcagcccttaaagatatttggacatgcagacttgataactaacgtttgcattcttgagacttacccaaaacccatacctcaagtccaagtttttagaattcatgaaataaagatctcagtgagtgcataaaattgcgcaccagaatcatatccgtatagacaagaacacatctactagaaaaataataaaccaacacaccaatgcaactgtgttttcttctgttttaaagtatgttgtctttgtatgcatgtttgcttcttcctttttttttttaacatcacagataaattcaactctcacctcaggttttattgagagaactgtcaatgtgacttggcctctgtctttctagtcccagaaagaattgcactgaaatctgagctcctgtaataaaaacaaccatttgctgagagtaattaacatactgaaagagattttcttagagtacacaatggtgacattatattgcctctttataaataactttctatctatttctgtggattattcctacaaagtacttttcatatgtccaatttcttttcttcccctacaactactgtctgaatactggctctgctatttgctgatatgattctcggcaagttgcctgcactttttaaactttatttcctcattcagaacatggggccatacataatacaactcacttcagtgttattggggaattaaacaaaaaatgcatgggaagcatttaacatagtgcctgacacaataatgagtactcagtagatgttagcttttattaatattgttgttgttatgtccagaaacactatacctccagaaaatcatgggtacttgctggggacattggggatatgcatgatttggaaaagaatgactgctttttttgcttagatgagaaatttttctaagccagactccttcaaatatgtaagattctgttgtggattcaaggactgaaagaattcttggccgagtgtggtggcttatccctgtaatcccagcattttgtgaggacaaggcaggaagattgcttgagtccaggagtttgaaaccagcctgcgcaacatggcgaaaccctgtctctacaaaaaatacaaacattagctcggagtgagtgctgacatgtgcctgtactcccagctactcagaaggctgagatgggaggatctcatgagcctggggagtttgaggcttcagtgagccgtgatgacaccgtactatactccactccagcctgggtgacagtgagaccctgcctcaaaaaacaaacaaacaaacaaacaaaacaaaattaatctttttgctgatgtcatgtcagcagtgtgtgttgaaggctgtaaagcagccatttgttcagtttatttttccattgaacaagtatttatcaaaaacatactttgtggcagtcactatgctaggagctatgaatacagaaggaaaagtaaatgctcttggatactacactccagttgtgataaaaaagaaaaaatgtattcttcaccaacttcaacatcttgatgtgcaaaaacataatacatgaattagatctacctaattacacagaattagaccaattgtttctggaattgtgggctcatatttttaataactgtcctcctgcctctctgtcgacaggttttataaatattcatttaattacacacacacacacgaacaattgactagtacttgctctcattcttctagatgtcatcacaatggatcccaaagacaattgttcaaaagatgcaaatggtaagcttttgtgtttttcccttcctcctgatcattttgttttgaacttctctggcttgaaaaatcagggaatggattttgctaggttggatgctgcagaatggacctagtgatattttaaattagtccctcattttctaggagttgtattaacaaacctaactactgctttggggtatgagatgactgtaaattagagagggtacagtggtatagtgatatgcttttaattatttcaaaaaaaagattttattcattcatgtgtcttttttctttttcttttcttttttttttttttttggacagagtcttgctctgtcacccaggctggagtgcggtggcagtatctcagctcaccacaacctccgcctcccggcttcaagtgattctcctgcctcagcttctcgagtagctgggactacaggcgcgtgccaccatgcccggctaatttttgtatttttagtagagttggggtttcaccatgttggccaggatggcctcgaatttgtgacctcgtgatctgccccctcgccctcccgaactgttgggattacaggcgtgagtcactgtgcccggcctcctgtcctgtcttttgtttaatgactgggaaaaacatgataccatgttgcttctcgagttgttttgttttagtctttggtctttgctagtagctaataacacgaactagtgtttatcaagtgctttttacacagaagggcttgggctgtgttctgcattttcttgtttaaccctcttaaaactcctataaaatggtacatatttttctcccaatttacagtccctttaaagcaaataattataaaaatccctatacatgtcacacagctagatctgggatttcaaatcaggccatcaaacaaagagtttatgtacttagtaagttttctgttctttttctacaatagagtcagatagcaagaaattaccaagccaggaacctgaaacaaaacggacatcatgtggggctgggtgggtgcatgggctttgcagactggactttcactccagctcttttaatgattaggtgtaagtgacctacattttgtgagcaacagttttctcatcagccaacaaagaataattacaccagattcacagttattgaagagataaaggcatgaatgtgagatgtctggcatagggcatctcatttagcagacacagaatgagtacttgtttctggctttttctctctacatatgcacaaagaatgcgactagaagcatgggctctagccctgctcaactttcctctatttccaataccaaggggctctgacttaggctgccacaccaggcaaggagggcagtaccacctcacttgaccaagggcagggagtcacggacacatcacttcttgagatccttttccacaccaaggactgatgtttctggaattctcactttatgaagacaaaacatataaatggaaattttctcaggtagagactcactcttgtagctcattgagtaggcactagtggtccacccccactgtctttacttattccttgacatcacatatctcttgcaaaacctcaaataatattaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatgtatcttattctgtaaaaagaacatgtgcaatttctcttagatacactactgctgcagctcacaaacacctctgcatattacatgtacctcctcctgctcctcaagagtgtggtctattttgccatcatcacctgctgtctgcttagaagaacggctttctgctgcaatggagagaaatcataa

SEQ ID NO: 50 - DNA, 9549 bp, Homo sapiens
  source 1..9549: mol_type = genomic DNA
ataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacaggtatgtgtttacacatatcatctgtcagaacacttctttgaaagtgaatgctgcattttttcctttcagtattaatgaaaaacataaatctttcttaaaaattgttacatttaatggtagcgtaaatgccctgctacttttctatagaattaaaatggtataggttttggagaaaacaaaattgaaaaagttgctgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaataatgtttatctgaagataactgtagatcatatttatctgtagataatgtttatctgtggagtgtggctctacaaaacatagaatagtcttggtcactgcagttttatagaggccttgggtttttcagagtttcattttatatatcaccataaagtaacatttcataattacaggttggtaaggcttacatgtacaaacattcttccattttccataataaatgcatttcctgccattggtgaatgcagctcaataaacatttattgtacaattatgacacgccaggcttagtggaaatgtggatgaacagacaaggatgagttactgtcctaaggatgatgcatgacagtgcagagaatatactctcttcctgatcactcagggtcactcatgattcatgcgcgaggtcccaaaacagtgcctttgatgcagattctgtacatctctagacgattggtccaagggctgaatgtgctctggcccagtggtccagtctgtcactatatgtcaacatcctgaatatgaacataacagtccaacatctcaagagtgggcatgaaaaggactcattttgtgctttttcctgtggttaacaagtcctttttagcctgggggaacaagcattaacaaaatgtttgaagatctttgccacgtaccattccaaatttctagggtaagtctttagcttttcagatcctgagtttctgcaatgatcaaatgtgatttggacagttgcgttgactttctcctggggctataatggagtgcaaaggaaacaatggcagggaaaatgcttgctttcaaaatggtagcatggatgtgttcattcgtgtagttactgtattaggtatagcctttcctgaaactaactgaagtggggttataaaaacagtcccaattttctatttcctttgctgagacacaaagaggagacaaaagagcaaagcttgagggtagttttaccactgtgcttaagtgttctgatttttccagtgatcagggtgaaataaaaagcatagtaagttccagggcagtgaataccatacaggagacaagttacagttttataatgtgttttactttacactaaattctaaaagtaaaatgtctttttttttttccgagacagagtttcactcttgtagcccaggcaggagtgctatggtgtgatctcggctcacagcaacctccacctcccagtttcaagcgattcttctgcctcagcctcccgagaagttgaaattacaggtgcctggcaccatatctcgctaattattctatttttagtagagatcgggttttaccatgttggccaggctggtctcgaactcctgacttcaagtgatccacccgcctcagcctcccaaagtgctgggattacaggtgtgagtcactgtgccggacctaacagtaaaatgtctttcatgtgcttctcaaggcaactacattaaggaggacacatctcttaatgtcattctacagtagatttctaatgctctttcttggaagtttgtttttctgagaagagctaaaaatataataacatggaagtgatcatattatataatcaatgaagtgctttcaaaggagataaaactaacctggtctgcatttgcaaccagccttgattgagagagagagaactcaggatacacttagagattttattatggggaatagttactttattcattttacctcaatcaatgcatggaaataagtgacagtcattttcatttatcttttaataaataaagtcaccatgaggaaaatgaaaacccattaaagtcagtccttaaagatatttggacatgcagacatgataactaacatttccattcgtgagacttacccaaaacctatacctcaagtccatttcttagaatacatgaaataaagatctcagtgagtgtataaaactgcacaccagaatcatatccgtatagacaagaatacatctactagaaaaatataaaccaaaacaccaaggtgactctgtttttttctgttttaaaatatgttgtctttgtatgcatgtttgcttcttcctttttttttttaaacatcgcagataaattcaactctcacctcagttgagagagaactgtcaatgtgacttggcctctctctttctagtcccagaaagaattgcactgaaatgctgagctcctgtaataaaaatgaccatttgctgagagtaattaacatactgaaagagattttcttagaatagtgcacaatggcccaatggtgacattatattgtctctttataaattattttctatctatttctgtggattatttctacaaagcacttttcatatgtccaattccttttattcccctacaagtactgactgactactggctctgctgttcactgatatgactttcggcaagttgcctgcactttttaaacgttatttcctcattcagaacatggggccatacaaaatacaactcacttcagtgttattggggaattaaacaaataaatgcatgggaagcatttaacatagtgcctgacacaataatgagcactcagtagatgttagcttttattaatattgttgttgctatgtccagaaacactatacctccagaaaatcatgggtacttgctggggacgttggggatatgcatgattttgaaaggagtgactgctctttactgctcagatgagaaatttttctaagccagactccttcaaacatgtaagattctgttgtggattctaggactgaaagaattcttggccgagtgtggtggcttatcctggtaatctcatcatttgggaggacaaggcaggaagattgcttgagcccaggagttggaaacaagcctggacaacatggcgaaaccctgtctctacaaaaaatacaaacattagctggtcatgggagtgagtgcctgtactcccagctactcaggaggctaagataggaggatcacctgagcctgggcagtttgaggtttcagtgagccgtgatgacaccatactatactccactccagcctgggtgacagtgacatcctgcctcaaaaaaacccccaaaattattctttttgctgatttcatgtcagcagtgtgtgctgaaggctgtaaagtagccacttgttctgtttatttttccattgaacaagtatttatcaaaaacgtactttgtggaaggcactgtgctaggaactatgcatacagaaggaaaaccaaatgttcttggatactacactccagttgtgataaaaaagaaaaaagtattcttcacaaacttcaacattttgatgtgcaaaaacataatatatgaattagatctacctaactacacagaattagaccaattatttctgggattatgggctcatatttttaataactgtcctcctacctctctgttgacaggttttataaatattcatttaattacacacagtcacagacacactcagacacacacacatacacacacacacacaccttgacaaataatgggcatgaacaattgactggtacttgctctcattcttctagatgtcaccacagtggatcccaaatacaattattcaaaggatgcaaatggtaagtttttgtgttttttatttcctcctgatcattttaagttttgaacttctctggcttgaaaaatcagggaatggattttgctaggttggatgctgcagaatggacctaatcatattttaaattagtccctctttttctaggagttgtattaacaaacctaactactgcttcatgtaagagatgactgtaaattgaagggtacagtgatatgctttcagttatttcaaaaaacagactttactcatccatgtgtcttttttcttttcttttttttcttttttgagacggagtctcgctctgttgaacaggctggattgcagtgacgcgatctcacctcactacaacctccgcctctggagttcaagcgattctccagcctcagcttctcaagtagctgggactacaggcacatgccaccatgtccgggtcatctttgtatttttagcagagaccgggtttcactatgttggccaggctggtctagaattcctgacttcgtgatctgccccctcagccctccgaagtgctgggattacagacgtgagtcactgtgcccggcctaacagtaaaatgtctttcatgcgcttctcaaggcaactacgttaaggaggacacttctcttaatgtcattctacagtagatttctaatgctctttcttggaagtttgtttttctgagaaaagctaaaaatataacatggaagtgatcatattgtataatcaatgaagtgcttttcaaggagataaaactaatctggtccacgtttgcaaccaaccttgattgagagagagagagaactcaggatacacttggagattttattatggggaatagttactttattcttttttcctcaatcaattcatggaaataagtgatagtcatattcatttatcttttaataaatgaagtcaccatgaggaaaataaaaagacattgaaaacccattaaagttagcccttaaagatatttggacatgcagacttgataactaacgtttgcattcttgagacttacccaaaacccatacctcaagtccatgtttttagaattcatgaaataaagatctcagtgagtgcataaaattgcgcaccagaatcatatccgtatagacaagaacacatctactagaaaaataataaaccaacacaccaatgcaactgtgttttcttctgttttaaaatatgttgtctttgtatgcatgtttgcttcttccttttttttttttaacatcacagataaattcaactctcacctcaggttttattgagagaactgtcaatgtgacttggcctctgtctttctagtcccagaaagaatcgcactgaaatgctgagctcctgtaataaaaatgaccatttgctgagagtaattaacatactgaaagagattttcttagagtacacaatggtgacattatattgtctctttataaataactttctatctatttctgtggattattcctacaaagtacttttcatatgtccagtttcttttcttcccctacaactaccgtctgaatactggctctgctatttgctgatatgattctcggcaagttgcctgcactttttaaactttatttcctcattcagaacatggggccatgtaatactcatgtacgtgagtattacgtaataatgctcacttaagtgttactggggaattaaacaaaaaaatgcatggcaagcatttaacatagtgcctgacacaataatgagcactcagtagatgttagattttattaatattgttgttgttatgtccggaaacactatacctccagaaaatcatgggtacttgcttgggatgttggggatatgcatgatttggaaaggtatgactgcttttttctgcttagatgagaaatttttctaagccagactccttcaaatatgtaagattctgttgtggattctaggacggaaagaattcttggtcaggtgtggtttcttatccctgtaatcccagaattttgggaggacaaggcaggaagattgcttgagcccaggagtttgaaaccagcctgggcaacaagacgaaaccctgtctctacaaaagtacataaattagcttggcttggtggtgtgtgcctgtattaccagctattcgggagactgagatgggaggatctcctgaacctgtgaagtttgaggcttcagtgagccgtgatgacaccatactatactcgactccagcctgtgcgacagtgagactctgcgtcaaaaaaaaaaccccaaaattattgtttttgctgatttcaggtcagcagtgtgtgctgaagggtgtaaagtagccacttgatcagtttatttttccactgaacaagtatttatcaaaaacatactttgtggtctgtttttgataaataaaaaggcactgtgctaggagccatgaatacagaaggaaaaccaaatgttcttggatactacactccagttgtgataaaaaagaaaaatgtattcttcacgaacttcaacattttgatatgcaaaaacatagtatataaattagatctacctgattacgtagaatcagaccaattatttctggaattgagggctcatatttttaataactgtcctcctgcctctctgttgacaggttttataaatattcatttaattacacacacacacacacacaccttgacaaataatggacatgaacaattgactagtacttgctctcattcttctagatgtcatcacaatggatcccaaagacaattggtcaaaagatgcaaatggtaagcttttgtgtttttcctttcctcctgatcattttaagttttgaacttctctggcttgaaaaatcagggaatgggccgggtgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcttagtggcgggcgcctgtagtcccagctacttgggaggctgaggcaggagaatggcgtgaacccgggaggcggagcttgcagtgagccgagattgcgccactgcactccactccagcctgggcgacagagcgagactccgtctcaaaaaaaaaaaaaaaaaaaaaaaaagaaaaatcagggaatggattttgctaggttggatgctgcagaatggacctagtgatattttaaattagtccctctttttctaggagttgtattaacaaacctaactactgcttcgggtatgagatgactgtaaattagagggtacagtgatatgctttcagttatttcaaaaaacagactttattcatccgtctgtcttttttttttttttttttttttttttttgagacggaggagtctcactctatcacccaggctggagtgcagtggcgcgatctcggctcaccataacctccgccttactggttcaagcgattctccagcctcagcttctcaagtagctgggactacaggtgcacaccaccatacctggctaatttttgtatttttaatagagatggggtttcaccacgctggccaggatggtcttgaattcttgacctcgtgatctgccccctcgggctcccaaacttctgggattataggcgtgagccactgtgcccggccttctgtcttttgttataatgactggggaaaacatgataccatgttgcttcttgagttgttttgttttagtctttggtctttgctagtagctaataacacgaactagtgtttatcaagtgctttttacacagaagggcttgttctgcattttctagtttaatcatcttaatactcctataaagtagtacaatatattttctcccattttacagtccctttaaagtaaataactataaaaatcccttatacatgtcacacagctaggtctggcatttcaaatcaggacatcaaacaaagaattcgtgcagttactaagtcctctattttttctacaatagaaaaaatagcaagaattacagatagcaagacattacaaggcaggaatctgaaacgaaagggacataatgtggggctgggtgggtgcatgagctttgcagactagactttcattccagctcttttaatgattaggtgtaagtgacctacattttgtgagtaacagttttctcatcagccaactaagaataattacaccagattcacagttattgaagagataagggcatgaatgtgagatgtctggcgtagggtatctcatttagcagacacagaatgaatacttgtttctggctttttctctctacatatgcacaaagaatgtgactagaagcattggctctagccctgctcaactttcctctatttccaataccaaggggctctgacttaggctgccacaccaggcaaggaggggcagtaccacctcacttgaccaagggcagggagtcacggacacatcacttcctgagatccttttccacaccaaggactgatgtttctggaattctcactttatgaagacaaaacatataaatggaaatttctgcaggaagagactcactcttgtagctcattgagtaggcactagtggtccacccccactgtctttacttattccttgacatcacatatctcttgtaaaacctcaaataatgttaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatatatcttattctgtaaaaagaacatgtgcaatttctcttagatacactactgctgcagctcacaaacacctctgcatattacacgtacctcctcctgctcctcaagagtgtggtctattttgccatcatcacctgctgtctgcttagaagaacggctttctgctgcaatggagagaaatcataa

SEQ ID NO: 51 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 52 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 53 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 54 - protein, 127 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 55 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 56 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 57 - protein, 309 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 58 - protein, 349 aa, synthetic construct
  REGION 1..349: note = Description of sequence: antibody component
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFRSKRSRLLHSDYMNMTPRRPGPTRKHYQPYAPPRDFAAYRKRGRKKLLYIFKQPFMRPVQTTQEEDGCSCRFPEEEEGGCEL

SEQ ID NO: 59 - protein, 19 aa, synthetic construct
  REGION 1..19: note = Description of sequence: antibody component
GGGGSGGGGSGGGSGGGGS

SEQ ID NO: 60 - protein, 24 aa, synthetic construct
  REGION 1..24: note = Description of sequence: antibody component
GGGGSGGGGSGGGGSGGGSGGGGS

SEQ ID NO: 61 - protein, 29 aa, synthetic construct
  REGION 1..29: note = Description of sequence: antibody component
GGGGSGGGGSGGGGSGGGGSGGGSGGGGS

SEQ ID NO: 62 - protein, 5 aa, synthetic construct
  REGION 1..5: note = Description of sequence: antibody component
GGGGS

SEQ ID NO: 63 - protein, 10 aa, synthetic construct
  REGION 1..10: note = Description of sequence: antibody component
GGGGSGGGGS

SEQ ID NO: 64 - protein, 10 aa, synthetic construct
  REGION 1..10: note = Description of sequence: antibody component
IPNIQNPDPA

SEQ ID NO: 65 - DNA, 30 bp, synthetic construct
  misc_feature 1..30: note = Description of sequence: DNA encoding antibody component
  source 1..30: mol_type = other DNA
attcccaatatccagaaccctgaccctgcc

SEQ ID NO: 66 - protein, 11 aa, synthetic construct
  REGION 1..11: note = Description of sequence: antibody component
LEDLKNVFPPE

SEQ ID NO: 67 - DNA, 33 bp, synthetic construct
  misc_feature 1..33: note = Description of sequence: DNA encoding antibody component
  source 1..33: mol_type = other DNA
ctggaggatctgaaaaacgtgttccctcctgaa

SEQ ID NO: 68 - RNA, 20 nt, synthetic construct
  misc_feature 1..20: note = Description of sequence: DNA encoding antibody component
  source 1..20: mol_type = other RNA
gggcttggtgatctgcctcg

SEQ ID NO: 69 - DNA, 420 bp, synthetic construct
  misc_feature 1..420: note = Description of sequence: DNA encoding antibody component
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 70 - DNA, 534 bp, synthetic construct
  misc_feature 1..534: note = Description of sequence: DNA encoding antibody component
  source 1..534: mol_type = other DNA
gatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 71 - DNA, 528 bp, synthetic construct
  misc_feature 1..528: note = Description of sequence: DNA encoding antibody component
  source 1..528: mol_type = other DNA
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 72 - protein, 296 aa, synthetic construct
  REGION 1..296: note = Description of sequence: antibody component
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - DNA, 888 bp, synthetic construct
  misc_feature 1..888: note = Description of sequence: DNA encoding antibody component
  source 1..888: mol_type = other DNA
caggtgcagctggtggagtctgggggaggcgtggtccagcctgggaggtccctgagactctcctgtgcagcctctggatttaccttcagtagctatattatgcactgggtccgccaggctccaggcaaggggctggagtgggtggcagttatatcatatgatggaagaaacaaatactacgcagactccgtgaagggccgattcaccatctccagagacaattccaagaacacgctgtatctgcaaatgaacagcctgagagctgaggacacggctgtgtattactgtgcgagagatacggatggctacgattttgactactggggccagggaaccctggtcaccgtcctggaggatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 74 - protein, 249 aa, synthetic construct
  REGION 1..249: note = Description of sequence: antibody component
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 75 - DNA, 747 bp, synthetic construct
  misc_feature 1..747: note = Description of sequence: DNA encoding antibody component
  source 1..747: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaccaactggccgctcactttcggcggagggaccaaggtggagatcattcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 76 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 77 - DNA, 762 bp, synthetic construct
  misc_feature 1..762: note = Description of sequence: DNA encoding antibody component
  source 1..762: mol_type = other DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 78 - DNA, 864 bp, synthetic construct
  misc_feature 1..864: note = Description of sequence: DNA encoding antibody component
  source 1..864: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 79 - protein, 116 aa, synthetic construct
  REGION 1..116: note = Description of sequence: antibody component
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTV

SEQ ID NO: 80 - protein, 106 aa, synthetic construct
  REGION 1..106: note = Description of sequence: antibody component
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEI

SEQ ID NO: 81 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 82 - DNA, 20 bp, Homo sapiens
  source 1..20: mol_type = genomic DNA
gggcttggtgatctgcctcg